(19) United States
(12) Patent Application Publication
(10) Pub. No.: US 2015/0072933 A1
(43) Pub. Date: Mar. 12, 2015

(54) NOVEL SEQUENCES FOR THE CONTROL OF REPRODUCTION IN FISH

(71) Applicants: Berta Levavi-Sivan, Rosh Haayin (IL); Chaim Gilon, Jerusalem (IL)

(72) Inventors: Berta Levavi-Sivan, Rosh Haayin (IL); Chaim Gilon, Jerusalem (IL)

(73) Assignee: Yissum Research Development Company of the Hebrew University of Jerusalem Ltd., Jerusalem (IL)

(21) Appl. No.: 14/236,063

(22) PCT Filed: Aug. 1, 2012
(86) PCT No.: PCT/IL2012/050287

§ 371 Date: Sep. 8, 2014

Related U.S. Application Data
(60) Provisional application No. 61/513,724, filed on Aug. 1, 2011.

(51) Int. Cl. C07K 14/575

(52) U.S. Cl. CPC C07K 14/575
USPC 514/9.8; 530/326

(57) ABSTRACT

The present invention relates to novel peptide sequences, compositions and methods for controlling fish reproduction. More particularly, the invention provides novel Neurokinin B peptides NKF and NKB and analogues thereof that regulate reproduction in fish. The invention further provides preprohormone thereof comprising at least one of a first peptide fragment of the amino acid sequence X¹-X²-X³-X⁴-X⁵-X⁶-Asp⁷-X⁸-Phe⁹-Val¹⁰-X¹¹-Leu¹²-Met¹³ and a second peptide fragment of the amino acid sequence of Glu¹-Met²-His³-Asp⁴-Ile⁵-Phe⁶-Val⁷-Gly⁸-Leu⁹-Met¹⁰ and variants thereof, nucleic acid sequences and novel fish NKB receptors.

### FIELD OF THE INVENTION

The invention relates to novel sequences, compositions and methods for controlling fish reproduction. More particularly, the invention provides novel Neurokinin B and Neurokinin receptor amino acid sequences, compositions thereof and methods using the same for controlling reproduction in fish.

### BACKGROUND OF THE INVENTION

Puberty comprises the physiological and behavioral changes that occur during the transition from juvenile life into sexual maturity and reproductive competence. In all vertebrates, puberty onset is usually initiated with the activation of the neuroendocrine reproductive axis. Despite the knowledge that both the gonadal hormone-dependent and independent processes underlying puberty contain neural components, the identity of the neuronal factors involved in these mechanisms is unknown. Humans bearing loss-of-function mutations of the genes encoding either neurokinin B (NKB) or its cognate receptor, neurokinin 3 receptor (NK3R), display hypogonadotropic hypogonadism [1]. This report implicates NKB signaling as an essential component for the onset of puberty and the control of gonadotropin secretion in fish reproduction. NKB and NK3R, which are encoded by the Tachykinin 3 gene and by the Tachykinin receptor 3 gene, respectively, are also termed Tac3 and Tac3r, respectively.

Studies provide evidence that a group of neurons in the hypothalamic infundibular/arcuate nucleus form an important component of human reproductive circuit. These neurons are steroid-responsive and co-express NKB, kisspeptin, dynorphin, NK3R and estrogen receptor α (ERα) in a variety of mammalian species. Compelling evidence in humans indicate that these neurons function in the hypothalamic circuitry regulating estrogen negative feedback on gonadotropin-releasing hormone (GnRH) secretion. Moreover, in rats, they form a bilateral, interconnected network that projects to NK3R-expressing GnRH terminals in the median eminence [2]. This network provides an anatomical framework to explain how coordination among NKB/kisspeptin/dynorphin/NK3R/ERα neurons may mediate feedback information from the gonads to modulate pulsatile GnRH secretion. There is indirect evidence that this network may be part of the neural circuitry known as the “GnRH pulse generator”, with NK3R signaling as an important component. This theory provides a compelling explanation for the occurrence of hypogonadotropic hypogonadism in patients with inactivating mutations in the NKB or its receptor, also termed TAC3 or TAC3R genes, respectively [2].

In teleosts, one of three infraclasses of the ray-finned fishes, to which most living fishes belong, the pituitary receives a direct innervation by neurons sending projections to the vicinity of the pituitary gonadotrophs. Among the neurotransmitters and neuropeptides released by these nerve endings are GnRH and dopamine, acting as stimulatory and inhibitory factors (in many but not all fish) on the liberation of Luteinizng hormone (LH) and Follicle-stimulating hormone (FSH) [3].

The activity of the corresponding neurons depends on a complex interplay between external and internal factors that will ultimately influence the triggering of puberty and sexual maturation. Among these factors are sex steroids and other peripheral hormones and growth factors, but little is known regarding their targets [3].

As noted above, Neurokinin B (NKB) is a member of the tachykinin family of peptides. Tachykinins are characterized by the common carboxyl-terminal amino-acid sequence Phe-X-Gly-Leu-Met-NH2 (X is a hydrophobic residue), also denoted by SEQ ID NO. 92, and include substance P (SP), neurokinin A (NKA) and NKB, as well as neuropeptide K, neuropeptide γ, and hemokinin-1 [5]. Tachykinins are produced from precursors by cleavage at designated sites (normally Lys and/or Arg), by specific enzymes that include the prohormone convertases. The products are further post-translationally modified by the action of carboxypeptidases that remove the COOH-terminal dibasic residues, allowing the action of peptidylglycine α-amidating enzyme that converts the exposed COOH-terminal glycine residue into an amide [6]. In mammals, NKB is the only tachykinin synthesized from the preprotachykinin-B gene [7] and is currently designated Tachykinin 3 gene (TAC3) in humans, Tac3 in nonhuman primates, cattle and dogs, and Tac2 in rodents.

After NKB binds its receptor, NK3R activation increases intracellular Ca2+ concentration through inositol phospholipid hydrolysis. Alternatively, NK3R activation can increase intracellular cAMP levels, through adenylate cyclase activation [8].

### REFERENCES

[1] Topaloglu, A. K. et al., Nat. Genet. 41(3):354-358 (2009)
[2] Rance, N. E. et al., Brain Research 1364:116-128 (2010)
[3] Levavi-Sivan, B. et al., Gen. Comp. Endocrinol. 165(3):412-437 (2010)
[4] Biran, J. et al., Biol Reprod 79(4):776-786 (2008)
[5] Page, N. M. Cellular and Molecular Life Sciences 61(13):1652-1663 (2004)
[6] Eipper B A, Stoffers D A, Mains R E Annu Rev Neurosci 15:57-85 (1992).
[7] Page, N. M. et al., Regulatory Peptides 98(3):97-104 (2001)
[8] Satake, H. and Kawada, T. Current Drug Targets 7:963-974 (2006)
[9] Levavi-Sivan, B. et al., Biol. Reprod. 70(6):1545-1551 (2004)
[10] Levavi-Sivan, B. et al., Biol Reprod 75:642-650 (2006)
[11] Tang, R. et al., Acta Biochim Biophys Sin 39(5):384-390 (2007)
[12] Palevitch, O. et al., Cell and Tissue Research 327(2):313-322 (2007)
[13] Mitani, Y. et al., Endocrinology 151(4): 1751-1759 (2010)
[14] Zhang, Y. Bmc Bioinformatics 9(1):40 (2008)
[15] Roy A. et al., Nat. Protocols 5(4):725-738 (2010)
[15] Heiden, T. K. et al., Toxicol Sci 90:490-499 (2006)
[16] Seidah, N. G. & Prat, A. Essays in biochemistry 38:79-94 (2002)
[17] Eipper, B. A. et al., Annual Review of Neuroscience 15(1):57-85 (1992)
[18] Page, N. M. et al., Peptides 30(8):1508-1513 (2009)
[19] Kitahashi, T. et al., Endocrinology 150(2):821-831 (2009)
[20] Aizawa, K. et al., Journal of Radiation Research 48(2):121-128 (2007)
[21] Bianco, I. et al., Neural Development 3(1):9 (2008)
[22] Roussigné, M. et al., Development 136:1549-1557 (2009)
[23] Concha, M. L. and Wilson, S. W. Journal of Anatomy 199(1-2):63-84 (2001)
[24] Berman, J. R. et al., Journal of Comparative Neurology 517(5):695-710 (2009)
[25] Alderman, S. L. and Bernier, N. J. Journal of Comparative Neurology 502(5):783-793 (2007)
[26] Lehman, M. N. et al., Endocrinology: en. 2010-0022 (2010)
[27] Mantha, A. K. et al., J Biomol Struct Dyn 22(2):137-148 (2004)
[28] Steven, C. et al., General and Comparative Endocrinology 133(1):27-37 (2003)
[29] Lee, Y. R. et al., Endocrinology 150(6):2837-2846 (2009)
[30] Cheng G. et al., Endocrinology 151:301-311 (2010)
[31] Southey, B. R. et al., Nucleic Acids Res 34(Web Server issue):W267-W272 (2006).
[32] Tamura, K., J. Dudley, et al. (2007). “MEGA4: Molecular evolutionary genetics analysis (MEGA) software version 4.0.” Mol Biol Evol 24(8): 1596-1599.
[33] Aizen, J. et al., (2012) Gen Comp Endocrinol 178:28-36.
[34] Aizen, J., H. Kasuto, et al. (2007). General and Comparative Endocrinology 153: 323-332.
[35] Taranger, G. L. et al., (2010) General and Comparative Endocrinology 165:483-515.

Acknowledgement of the above references herein is not to be inferred as meaning that these are in any way relevant to the patentability of the presently disclosed subject matter.

### SUMMARY OF THE INVENTION

According to a first aspect, the invention relates to an isolated preprohormone peptide or any active hormone peptide derived therefrom regulating reproduction in fish. In more specific embodiments the preprohormone of the invention comprises a first and a second peptide fragments:

(a) the first peptide fragment comprises the amino acid sequence of:
X¹X²-X³-X⁴-X⁵-X⁶-Asp⁷-X⁸-Phe⁹-Val¹⁰-X¹¹-Leu¹²-Met¹³, as denoted by SEQ ID NO. 96.

### Wherein:

X¹, is Tyr or any hydrophobic, a polar or positively charged amino acid selected from Phe, Ser, Lys and Gln;
X², is Asn or Ser or a hydrophobic, a polar, an acidic or positively charged amino acid selected from His, Thr, Asp, Arg, Lys and Ile;
X³, is Asp or a hydrophobic or a polar amino acid selected from Phe and Arg;
X⁴, is Ile or Leu or a polar or a hydrophobic amino acid selected from Asp, Tyr and Val;
X⁵, is Asp or an hydrophobic amino acid Met;
X⁶, is Tyr or acidic amino acid selected from Asp or His;
X⁸, is Ser or a polar or hydrophobic amino acid selected from Thr, Phe and Val;
X¹¹, is Gly or a polar amino acid, Ser;
X¹³, is Met or a hydrophobic amino acid Leu, and
(b) the second peptide fragment comprises the amino acid sequence of:
Glu¹-Met²-His³-Asp⁴-Ile⁵-Phe⁶-Val⁷-Gly⁸-Leu⁹-Met¹⁰ as denoted by SEQ ID NO. 40 and variants thereof. More specifically, the variants may comprise a substitution in at least one position selected from a group consisting of:
Glu¹ may be any one of Asn, Asp and Tyr; His³ may be substituted with any one of Asn and Asp; Asp⁴ may be Gln; Ile⁵ may be substituted with Val; Phe⁶ may be Leu; Val⁷ may be Ile; Gly⁸ may be substituted with Ala; and Met¹⁰ may be replaced with Leu.

It should be noted that in certain embodiments, the active hormone peptide derived from the preprohormone of the invention comprises at least one of the first or the second peptide fragments according to the invention.

According to a second aspect, the invention relates to an isolated peptide at least one of:

(a) a first peptide fragment comprises the amino acid sequence of:
X¹-X²-X³-X⁴-X⁵-X⁶-Asp⁷-X⁸-Phe⁹-Val¹⁰-X¹¹-Leu¹²-Met¹³, as denoted by SEQ ID NO. 96;

### Wherein:

X¹, is Tyr or any hydrophobic, a polar or positively charged amino acid selected from Phe, Ser, Lys and Gln;
X², is Asn or Ser or a hydrophobic, a polar, an acidic or positively charged amino acid selected from His, Thr, Asp, Arg, Lys and Ile;
X³, is Asp or a hydrophobic or a polar amino acid selected from Phe and Arg;
X⁴, is Ile or Leu or a polar or a hydrophobic amino acid selected from Asp, Tyr and Val;
X⁵, is Asp or an hydrophobic amino acid Met;
X⁶, is Tyr or acidic amino acid selected from Asp or His;
X⁸, is Ser or a polar or hydrophobic amino acid selected from Thr, Phe and Val;
X¹¹, is Gly or a polar amino acid, Ser;
X¹³, is Met or a hydrophobic amino acid Leu, and
(b) a second peptide fragment comprises the amino acid sequence of:
Glu¹-Met²-His³-Asp⁴-Ile⁵-Phe⁶-Val⁷-Gly⁸-Leu⁹-Met¹⁰ as denoted by SEQ ID NO. 40 and variants thereof. More specifically, the variants may comprise a substitution in at least one position selected from a group consisting of:
Glu¹ may be any one of Asn, Asp and Tyr; His³ may be substituted with any one of Asn and Asp; Asp⁴ may be Gln; Ile⁵ may be substituted with Val; Phe⁶ may be Leu; Val⁷ may be Ile; Gly⁸ may be substituted with Ala; and Met¹⁰ may be replaced with Leu. It should be noted that the peptide of the invention regulates reproduction in fish.

Another aspect of the invention relates to a polynucleotide sequence encoding the preprohormone peptide of the invention.

According to another aspect, the invention relates to a composition regulating reproduction in fish. The composition of the invention comprises any of the active hormone peptides of the invention as well as analogues thereof, the preprohormone peptides of the invention and polynucleotide sequences encoding the same.

According to a fifth aspect, the invention provides a method for regulating reproduction in fish. The method of the invention comprises the step of administering to a treated fish an effective amount of at least one of: the isolated active hormone peptides of the invention as well as any analogues thereof, the isolated preprohormone peptide of the invention, any nucleic acid sequence encoding said preprohormone, any combinations thereof and any composition comprising the same.

In yet another aspect, the present invention provides the use of an effective amount of at least one of any of the active hormone peptide of the invention, any analogues thereof, an isolated preprohormone, that is the precursor peptide of the invention, or any nucleic acid sequence encoding the preprohormone molecule of the invention, and any combinations thereof, in the preparation of a composition for regulating reproduction in fish.

In another aspect, the present invention provides at least one of: any isolated active hormone peptide according to the invention, any analogues thereof, any isolated preprohormone that is a precursor of said active peptides, or any nucleic acid sequence encoding the preprohormone molecule of the invention, and any combinations thereof for use in a method for regulating reproduction in fish.

These and other aspects of the invention will become apparent by the hand of the following Figures.

### BRIEF DESCRIPTION OF THE FIGURES

In order to understand the disclosure and to see how it may be carried out in practice, embodiments will now be described, by way of non-limiting example only, with reference to the accompanying figures, in which:

FIG. 1A-1B. is a schematic presentation of nucleotide and deduced amino acid sequences of the zebrafish tac3a (FIG. 1A) and tac3b (FIG. 1B). Numbering of the deduced amino acid sequences begins with the first methionine of the ORF to the right of each line. Nucleotide numbers are to the left of each line. The start and stop codons are fraimed, signal peptide amino acids are underlined (as defined by SignalP program analysis http://www.cbs.dtu.dk/services/SignalP/), and the putative secreted peptides are underlined (nucleotides) and bold (amino acids).

FIG. 2A-2B. Unrooted phylogenetic tree of neurokinin

FIG. 2A. Shows a schematic presentation of unrooted phylogenetic tree of neurokinin sequences. The sequences cloned in this study are marked in bold sequences generated with both neighbor-joining (ClustalW 2.1) and maximum likelihood (Phylip 3.69, ProML) on the basis of alignments performed both by ClustalW and Muscle (3.8.31). Trees were visualized with FigTree (1.3.1). Gene nomenclature has been standardized to tac3, and species are indicated for illustration and comparison. Numbers at nodes indicate the bootstrap values from 1,000 replicates. (Scale bar indicates the substitution rate per residue.) GenBank accession numbers: Ligands: Danio rerio, zebrafish, tac3a (JN392856); zebrafish, tac3b (JN392857); Pimephales promelas, fathea minnow tac3 (BK008100); Ictalurus punctatus, channel catfish, tac3 (BK008101); Salmo salar, Atlantic salmon, tac3a (BK008102); Atlantic salmon, tac3b (BK008103); Dissostichus mawsoni, Antarctic toothfish, tac3 (BK008104); Sebastes rastrelliger, grass rockfish, tac3 (BK008105); Gadus morhua, Atlantic cod, tac3 (BK008107). Boreogadus saida, Arctic cod, tac3 (BK008109); Xenopus tropicalis, western clawed frog, tac3 (BK008110); Osmerus mordax, rainbow smelt, tac3 (BK008111); Oryzias latipes, medaka, tac3 (BK008114); Alligator mississippiensis, American alligator, tac3 (BK008115); Dicentrarchus labrax, European seabass, tac3 (BK008116); zebrafish, tact (BK008124); Gallus gallus, chicken, tac1 (BK008126); Sebastes rastrelliger, grass rockfish, tac1 (K008106); Oncorhynchus mykiss, rainbow trout, tac1 (BK008119); Salvelinus fontinalis, brook trout, tac1 (BK008120); Anoplopoma fimbria, sablefish tac (BK008121); Sebastes caurinus, copper rockfish, tac1 (BK008122); Carassius auratus goldfish tac1 (AAB86991.1); rainbow smelt, tac4a (BK008112); rainbow smelt, tac4b (BK008113); Gasterosteus aculeatus, three-spined stickleback, tac4 (BK008117); rainbow trout, tac4 (BK008118); Sus scrofa, pig, Tac4 (BK008123); zebrafish tac4 (BK008125); Arctic cod, tac4 (BK008108); zebrafish, tac 4 (BK008125); catfish tac1 (NP_{—}001187697); salmon tac1a (ACI67317); salmon, tac1b (ACI68385); frog, tact (NP_{—}001165757.1); Japanese medaka, tac1 (BAH03329); rainbow smelt, tac1 (AC010148.1); human, TAC1g (NP_{—}054703.1); human, TAC3a (NP_{—}037383.1); human, TAC4a2 (NP_{—}001070974.1); rabbit, Tac4 (NP_{—}001075634.1); mouse, Tac4 (NP_{—}444323.1); rat, Tac4 (NP_{—}758831.1); mouse, Tac1 (AAI44738.1); cow, Tac1 (AAI42366.1); cow, Tac3 (NP_{—}851360.1); pig, Tac3 (NP_{—}001007197.1); mouse, Tac3 (NP_{—}033338.2); rabbit and Tac1 (NP_{—}001095168.1). Abbreviations: mo. (mouse), ra (rat), pi (pig), hu (human), ra tro. (rainbow trout), ra. sm (rainbow smelt) ar. Co. (arctic code), zf (zebrafish), gol. (gold fish), sal. (salmon), cf (catfish), fr. (frog), ch. (chicken), co. (cow), rab. (rabbit), al. (alligator), E. sb. (European sea bass), fat. Min. (fathead minnow), med. (medaka), tf. (toothfish), g. roc. (grass rockfish), At. Co. (atlantic code), FIG. 2B. is a schematic presentation of gene organization of zebrafish tac3a and tac3b. Each gene is consisting of seven exons and code to both NKF and NKB. SP, signal peptide; NKF, neurokinin F; NKB, neurokinin B; ATG and TGA, start and stop codon, respectively. Abbreviations: Ex. (Exon), co. (coding), by (base pair).

FIG. 3A-3B. is a schematic presentation of nucleotide and deduced amino acid sequences of the zebrafish tac3ra (FIG. 3A) and tac3rb (FIG. 3B). Numbering of the deduced amino acid sequences begins with the first methionine of the ORF to the right of each line. Nucleotide numbers are to the left of each line. The start and stop codons are shaded in gray. Open circles, putative N-glycosylation sites; open squares, putative protein kinase C phosphorylation sites; open triangle, putative cAMP and cGMP-dependent protein kinase phosphorylation site; open diamonds, putative Casein kinase II phosphorylation sites; open trapezoid, putative tyrosine kinase phosphorylation site; open octagons, putative N-myristoylation sites. Predicted transmembrane domains (TM1-TM7) are underlined; arrowheads indicate the exon-intron boundaries.

FIG. 4. is a schematic presentation of unrooted phylogenetic tree of neurokinin receptor sequences. The sequences cloned in this study are marked in bold sequences generated with both neighbor-joining (ClustalW 2.1) and maximum likelihood (Phylip 3.69, ProML) on the basis of alignments performed both by ClustalW and Muscle (3.8.31). Trees were visualized with FigTree (1.3.1). Gene nomenclature has been standardized to tac3, and species are indicated for illustration and comparison. Numbers at nodes indicate the bootstrap values from 1,000 replicates. (Scale bar indicates the substitution rate per residue.) GenBank accession numbers: Receptors: zebrafish, tac3ra (JF317292); zebrafish tac3rb (JF317293); zebrafish tacr3c (XP_{—}002666594); Japanese medaka, tac3ra (BK008087); Japanese medaka, tacr3b (BK008088); Takifugu rubripes, fugu, tacr3a (BK008092); fugu tacr3b (BK008093); Tetraodon nigroviridis, spotted green pufferfish tacr3a (BK008096); spotted green pufferfish, tacr3b (BK008097); medaka, tacr1a (BK008089); medaka, tacr1b (BK008090); fugu, tacr1a (BK008095); spotted green pufferfish, tacr1a (BK008099); medaka, tacr2 (BK008091); fugu, tacr2 (BK008094); spotted green pufferfish, tacr2 (BK008098); zebrafish, tacr1a (XP_{—}001343073); zebrafish, tacr1b (XP_{—}692469); zebrafish, tacr2 (XP_{—}001341981.1); human, TACR1 (NP_{—}001049.1); human, TACR2 (NP_{—}001048.2); human TACR3 (NP_{—}001050); chicken, tacr3 (XM_{—}001232173); chicken, tacr2 (XP_{—}001232177.1); chicken, tacr1 (NP_{—}990199.1); Neoceratodus forsteri, lungfish, tacr1 (AAZ82194.1); fugu, tacr1b (AAQ02694.1); Octopus vulgaris, octopus, tkr (BAD93354.1); spotted green pufferfish, tacr1b (CAG05392.1) frog, tacr1 (NP_{—}001106489.1) frog, tacr3 (XP_{—}002934808.1); cow Tacr3 (NP_{—}001179262.1); cow, Tacr1 (XP_{—}002691234.1); cow Tacr2 (NP_{—}776894.1); rabbit, Tacr3 (NP_{—}001075524.1); rabbit, Tacr1 (XP_{—}002709748.1); rabbit, Tacr (NP_{—}001075800.1); mouse, Tacr3 (NP_{—}067357.1); mouse Tacr2 (NP_{—}033340.3); mouse, Tacr1 (NP_{—}033339.2); Caenorhabditis elegans, C. elegans, tkr (NP_{—}500930.1); Ciona intestinalis, ciona, tkr (NP_{—}001027809.1). Abbreviations: c. el. (c. elegance), oc. (octopus), ci. (cicna), zf. (zebrafish), med. (medaka), te. (tetradon), mo. (mouse), ra (rat), ch. (chicken), co. (cow), rab. (rabbit), fr. (frog).

FIG. 5A-D. Are schematic presentations of chromosomal locations of zebrafish tac3 and tac3 receptors in various vertebrate species. Genes adjacent to tac3 and tac3r in different genomes are shown. The genes are named according to their annotation in the human genome. FIG. 5A shows a comparison between zebrafish tac3a and human. FIG. 5B shows a comparison between zebrafish tac3b and medaka tac3. FIG. 5C shows a comparison between human, zebrafish and medaka tac3ra. FIG. 5D shows a comparison between zebrafish, green spotted pufferfish, and fugu tac3rb. Abbreviations: zf. (zebrafish), hu. (Human), chr. (chromosom), sg (syntenic gene), nsg (non-syntenic gene), ge. mis. F. reg. (Genes missing in fish in this region).

FIG. 6A-6B. is a graphical presentation of the expression of zebrafish mRNAs, as determined by real-time PCR. FIG. 6A is a graphical presentation of the expression of zebrafish tac3a, tac3b, tac3ra, or tac3rb mRNAs in various parts of the brain. FIG. 6B is a graphical presentation of changes in the expression of zebrafish tac3a at various ages toward puberty. The relative abundance of the mRNAs was normalized to the amount of elongation factor 1 α (ef1α) by the comparative threshold cycle method; the comparative threshold reflects the relative amount of the transcript. Results are means±SEM (n=11-15). Means marked with different letters differ significantly (P<0.05). Abbreviations: F. br. (forebrain), M.br. (midbrain), H.Br. (hindbrain), Pit. (pituitary), ov. (ovary), tes. (testis), Ag. We (age in weeks).

FIG. 7. is a graphical presentation of a localization experiment performed by real-time PCR of zebrafish tac3a, tac3b, tac3ra, and tac3rb mRNA in various tissues. The relative abundances of the mRNAs were normalized to the amount of elongation factor 1-α (ef1α) by the comparative threshold cycle method, where the comparative threshold reflects the relative amount of the transcript. Abbreviations: Ant. Intest+ panc. (anterior intestine and pancreas), post. Intes, (posterior intestine), Mus. (muscles), kid. (kidney), ad. (adipose), liv. (liver), ret. (retina).

FIG. 8A-8O. are microscope images showing the localization of tac3a during early stages of development, as detected by whole-mount ISH. Dorsal view of larva heads, anterior is to the left (FIG. 8A-E). High magnification of boxed area in Upper panel. Lateral view of larva heads (K-O). rHbn, right habenula; MB, midbrain; HB, hindbrain. FIG. 8P-8R are microscope images showing the localization of tac3a in adult zebrafish brain as indicated by ISH. ventral (Hav); medial (Ham) habenula (FIG. 8P) periventricular nucleus of posterior tuberculum (TPp); dorsal (Hd); ventral zone (Hv) of periventricular hypothalamus (FIG. 8Q) posterior tuberal nucleus (PTN); central zone (Hc) of periventricular hypothalamus (FIG. 8R). Magnification: A-E and K-O, 40×; F-J, 120×.

FIG. 9A-9G. is a graphical presentation of ligand selectivity of the NKB receptors, human NKBR (A and D) zebrafish Tac3ra (FIGS. 9B and 9E) or zebrafish Tac3rb (C and F), each together with SRE-Luc (FIG. 9A-9C) or CRE-Luc (FIG. 9D-9F) reporter genes. The cells were treated with various concentrations of human (hu) NKB; zebrafish (zf) NKBa; zfNKBb; Senktide or zfNKF. Data are expressed as the increase in luciferase activity over basal activity. Each point was determined in quadruplicate and is given as a mean±SEM. (FIG. 9G) presents a ribbon representation of human and zebrafish NKBs structural model. The PDB ID for the human structure is 1p9f. Abbreviations: pep. (peptide), ac. (activity).

FIG. 10A-10C.

FIG. 10A, 10B are graphical presentations of exposure of prepubertal fish to estradiol (18 nM) by immersion. FIG. 10C is a graphical presentation of intraperitoneal injection of sGnRHa, zfNKBa, zfNKBb, zfNKF, or senktide to mature zebrafish. Hormone values are means±SEM. Statistical significance vs. corresponding control values: **P<0.01; *P<0.05. Abbreviations: F. Exp. Fold expression), Lig. (ligand), Rec. (erceptor), Bas (basal), con. (control).

FIG. 11A-11F. is a graphical presentation of ligand selectivity of the NKB receptors to native and NKB-analog. Human (FIG. 11C, 11F), zebrafish tac3ra (FIG. 11A, 11D) or zebrafish tac3rb (FIG. 11B, 11E), each together with SRE-Luc (FIG. 11A-11C) or CRE-Luc (FIG. 11D-11F). The cells were treated with various concentrations of human NKB, zebrafish (zf) NKBa, zfNKBb or zfNKF or with their corresponding analogues. Data are expressed as the change in luciferase activity over basal activity and are from a single experiment, representative of a total of three such experiments. Each point was determined in quadruplicate and is given as a mean±SEM. Abbreviations: pep. (peptide), ac. (activity), rat. (ratio).

FIG. 12A-12B. is a graphical presentation of the levels of FSH and LH in the presence of NKB analogues in juvenile tilapia females, measured by ELISA. FIG. 12A is a graphical presentation of the level of FSH and FIG. 12B is a graphical presentation of the level of LH, both in the presence of the NKBa analogue (SEQ ID NO.44) or the NKF analogue (SEQ ID NO.46), at the indicated amounts. BW, body weight; FSH, follicle-stimulating hormone; LH, luteinizing hormone. Abbreviations: Ba (basal), T(h) (time in hour).

FIG. 13A-13B. is a graphical presentation of the levels of FSH and LH in the presence of NKB analogues in tilapia pituitary cells. FIG. 13A is a graphical presentation of the level of FSH and FIG. 13B is a graphical presentation of the level of LH, both in tilapia pituitary cells stimulated with 10 nM of the NKBa (SEQ ID NO.44) or NKF analogue (SEQ ID NO.46). GnRH was used as a control. FSH, follicle-stimulating hormone; LH, luteinizing hormone. Abbreviations: pep. Con. (peptide concentration), ana (analogue).

FIG. 14. is a graphical presentation of the level of LH in the presence of NKB analogues in mature carp, measured by ELISA. Mature female carp, were injected with the NKB analogues, namely NKBa (SEQ ID NO.44), NKBb (SEQ ID NO.45) and NKF (SEQ ID NO.46) at the indicated dose. Saline injection was used as a control. LH, luteinizing hormone. Abbreviations: Ba (basal), T(h) (time in hour), sal. (saline).

### DETAILED DESCRIPTION OF THE INVENTION

### Abbreviations:

neurokinin—NKB
neurokinin receptor—NKBR
saline sodium citrate—SSC
hour—h
expressed sequence tag—EST

### National Center for Biotechnology Information—NCBI

### Polymerase Chain Reaction—PCR

Days post fertilization—dpf

In recent years the world has witnessed an alarming decline in commercial fisheries, the result of over fishing of wild fisheries stocks and indirectly, the failure of commercial aquaculture to meet the demand for fisheries products (i.e., by a sufficient increase in commercially farmed fish products). According to the Food and Agriculture Organization (FAO) of the United Nations, nearly 70% of the world's commercial marine fisheries species are now fully exploited, overexploited or depleted. Based on anticipated population growth, it is estimated that the world's demand for seafood will double by the year 2025. Therefore, a growing gap is developing between demand and supply of fisheries products, which results in a growing seafood deficit. Even the most favorable estimates project that in the year 2025 the global demand for seafood will be twice as much as the commercial fisheries will harvest.

Worldwide, it is estimated that in order to close the increasing gap between demand and supplies of fisheries product, aquaculture will need to augment production five-fold during the next two and half decades. While there is a need to increase global aquaculture production, it is clear that fish farming must develop as a sustainable industry without having an adverse impact on the environment.

Still further, many of the economically important fish do not reproduce spontaneously in captivity. This is the case with mullet (Mugil cephalus), rabbitfish (Siganus sp), milkfish (Chanos chanos), striped bass (Morone saxatilis), sea bass (Dicentrarchus labrax), seabream (Sparus aurata), catfish (Clarias sp.) and many others. In all these species the reproductive failure is located in the female: whereas vitellogenesis is completed, the stages that follow, namely oocyte maturation and ovulation, do not occur, and thus there is no spawning. Instead, vitellogenic follicles undergo rapid atresia.

In some fish species which do ovulate spontaneously in captivity, such as trout and salmon, both Atlantic and Pacific, e.g. Atlantic salmon (Salmo salar) and Pacific salmon (Onchorhynchus sp.), ovulation is not synchronized and thus egg collection is a very laborious task. Additionally, the subsequent hatching of the fingerlings is not synchronized and therefore the ability to create schools of fingerlings being all at about the same growing stage, which is necessary for economically feasible fish farming, becomes very difficult.

In fish indigenous to temperate zones, such as seabream, seabass, striped bass, cyprinids and salmons, reproduction is seasonal, i.e. ovulation and subsequent spawning occur once or several times during a limited season. Inducing such fish to ovulate and spawn out of the natural spawning season might largely contribute to the management of fish farming

For one, out of season egg production may enable full utilization of the fish farm throughout the whole year, by making it possible to have at any given time fish of all ages. Overcoming the restrictions of seasonal spawning, therefore, may enable the marketing of adult fish year round.

Therefore, regulation of reproduction in fish is desirable for advancing and synchronizing the onset of puberty and enriching aquaculture. The inventors have now identified the gene system neurokinin B (NKB) and neurokinin receptor (NKBR), encoded by the Tachykinin 3 and the Tachykinin receptor 3 genes, respectively, in variety of fish species. The inventors have demonstrated the involvement of the NKB/NKBR system in controlling reproduction in fish by showing that neurokinin B is expressed in tissues that are involved in reproduction and that the level of NKB is increased toward puberty and in response to estradiol treatment.

The inventors have surprisingly found, that unlike in mammalian NKB proteins, the amino acid sequence of different species of fish includes two tachykinin (neurokinin) peptide sequences. More specifically, in addition to the NKB hormone peptide, only the fish preprohormone included a unique NKF hormone peptide. Both peptides, as well as analogues thereof, were shown by the inventors to induce signal transduction by their cognate receptors, NKBR (Neurokinin B receptor). Moreover, both peptides, as well as analogues thereof, were shown to elicit a significant LH and FSH secretion in sexually mature female zebrafish, carp and tilapia. Thus, the NKB/NKBR system is suggested as a useful means for controlling reproduction in fish and for improving aquaculture.

Thus, according to a first aspect, the invention relates to an isolated preprohormone peptide or any active hormone peptide derived therefrom regulating reproduction in fish. In more specific embodiments, the preprohormone of the invention comprises a first and a second peptide fragments:

(a) the first peptide fragment comprises the amino acid sequence, or the amino acid sequence of:
X¹-X²-X³-X⁴-X⁵-X⁶-Asp⁷-X⁸-Phe⁹-Val¹⁰-X¹¹-Leu¹²-Met¹³, as denoted by SEQ ID NO. 96, Wherein:
X¹, is Tyr or any hydrophobic, a polar or positively charged amino acid selected from Phe, Ser, Lys and Gln;
X², is Asn or Ser or a hydrophobic, a polar, an acidic or positively charged amino acid selected from His, Thr, Asp, Arg, Lys and Ile;
X³, is Asp or a hydrophobic or a polar amino acid selected from Phe and Arg;
X⁴, is Ile or Leu or a polar or a hydrophobic amino acid selected from Asp, Tyr and Val;
X⁵, is Asp or an hydrophobic amino acid Met;
X⁶, is Tyr or acidic amino acid selected from Asp or His;
X⁸, is Ser or a polar or hydrophobic amino acid selected from Thr, Phe and Val;
X¹¹, is Gly or a polar amino acid, Ser;
X¹³, is Met or a hydrophobic amino acid Leu, and
(b) the second peptide fragment comprises the amino acid sequence of:
Glu¹-Met²-His³-Asp⁴-Ile⁵-Phe⁶-Val⁷-Gly⁸-Leu⁹-Met¹⁰ as denoted by SEQ ID NO. 40 and variants thereof. More specifically, the variants may comprise a substitution in at least one position selected from a group consisting of:
Glu¹ may be any one of Asn, Asp and Tyr; His³ may be substituted with any one of Asn and Asp; Asp⁴ may be Gln; Ile⁵ may be substituted with Val; Phe⁶ may be Leu; Val⁷ may be Ile; Gly⁸ may be substituted with Ala; and Met¹⁰ may be replaced with Leu.

It should be noted that in certain embodiments, the active hormone peptide derived from the preprohormone of the invention comprises at least one of the first or the second peptide fragments according to the invention. It is to be understood that the active hormone peptide regulates different stages in fish reproduction, as will be disclosed herein after.

The invention provides preprohormone peptide molecules comprising two peptide fragments (referred to herein as the first and second peptide fragments). As known in the art, the term “preprohormone” used herein, refers to a precursor polypeptide of one or more prohormones, which are in turn precursors to peptide hormones. In general, the polypeptide comprises the peptide hormone or hormones, as well as superfluous amino acid residues that were needed, for example, to direct folding of the hormone molecule into its active configuration but have no function once the hormone folds. Other intervening amino acids include, for example, signal sequences. Hormones are eventually cleaved off from their precursor polypeptides by enzymatic activity. It should be noted that the term hormone as used herein refers to a substance, specifically, peptide, produced by one gland or organ of the body that then travels through the bloodstream to affect other tissues, organs, etc. The hormone peptides of the invention regulate the reproductive cycle in fish.

As indicated above, the invention provides a preprohormone polypeptide and any hormone peptide derived therefrom, being a mature form of the preprohormone. Thus, in certain embodiments, the invention relates to any mature and active hormone peptide cleaved of the preprohormone. In specific embodiments, the invention encompasses any active hormone peptide cleaved of the preprohormone of the invention by way of enzymatic cleavage.

According to some embodiment, the first peptide fragment comprised within the preprohormone peptide of the invention may comprise an N′ terminal signature of Asp-Ile-Asp (DID), as indicated by the amino acid sequence of:

X¹-X²-Asp³-Ile⁴-Asp⁵-X⁶-Asp⁷-X⁸-Phe⁹-Val¹⁰-X¹¹-Leu¹²-Met¹³, as denoted by SEQ ID NO. 96, Wherein:
X¹, is Tyr or any hydrophobic, a polar or positively charged amino acid selected from Phe, Ser, Lys and Gln;
X², is Asn or Ser or a hydrophobic, a polar, an acidic or positively charged amino acid selected from His, Thr, Asp, Arg, Lys and Ile;
X³, is Asp or may be substituted with any one of Phe and Arg;
X⁴, is Ile or may be replaced with any one of Leu, Asp, Tyr and Val;
X⁵, is Asp or may be substituted with Met;
X⁶, is Tyr or acidic amino acid selected from Asp or His;
X⁸, is Ser or a polar or hydrophobic amino acid selected from Thr, Phe and Val;
X¹¹, is Gly or a polar amino acid, Ser;
X¹³, is Met or a hydrophobic amino acid Leu.

Tachykinins are generally characterized by the common carboxyl-terminal amino-acid sequence Phe-X-Gly-Leu-Met-NH₂ (X is a hydrophobic residue), as noted in the Background section of the invention, above, by analyzing the amino acid sequence of a plethora of neurokinin genes in various fish (and other) species, searching for the characterizing Phe-X-Gly-Leu-Met-NH2 signature (also denoted by SEQ ID NO. 92). The inventors have surprisingly found that only the fish neurokinin B preprohormone comprise two separate neurokinin peptide fragments, namely a first and a second neurokinin peptide fragments, as detailed herein above.

Thus, according to more specific embodiments, the first peptide fragment comprised within the preprohormone peptide of the invention may comprise an amino acid sequence as denoted by any one of SEQ ID NO. 42, 43, 53, 57, 61, 65, 68, 72, 76, 98, 100, 102, 103 and 105, or any analogs or derivatives thereof.

In yet another specific embodiment, the second peptide fragment comprised within the preprohormone peptide of the invention may comprise the amino acid sequence as denoted by any one of SEQ ID NO. 40, 41, 54, 58, 62, 69, 73, 77, 93, 99, 101, 104 and 106, or any analogs or derivatives thereof.

As noted above, the preprohormone peptide of the invention, that may be also referred to as the “precursor” polypeptide, comprises both the first and the second peptide fragments and was identified as the Neurokinin preprohormon, specifically, Neurokinin preprohormone B. As shown by Example 1, the inventors isolated and identified different Neurokinin B preprohormone peptides from more then fourteen different species of fish.

Both, the first and the second different peptides indicated herein above are comprised within the preprohormone peptides of the invention forming different preprohormone from different fish species. Therefore, According to some specific embodiments, the preprohormone peptide of the invention, is translated from the tac3a cDNA and is therefore designated NKBa (Neurokinin Ba). Thus, in more specific embodiments, the preprohormone peptide of the invention may comprise an amino acid sequence as denoted by any one of SEQ ID NO: 49, 52, 56, 60, 67, 71, 75, 79, 81, 83, 85, 89, 87, and 91 or any analogues and derivatives thereof.

According to one specific embodiment, the preprohormone of the invention may be the zebrafish NKBa that comprises the amino acid sequence of SEQ ID NO. 49, or any homologs, fragments and derivatives thereof.

In certain embodiments, the preprohormone peptide of the invention comprises as a first peptide fragment, the NKFa first fragment that comprises an amino acid sequence as denoted by SEQ ID NO. 42 or any analogues and derivatives thereof.

In yet another embodiment, the preprohormone of the invention comprises a second peptide fragment that comprises an amino acid sequence as denoted by SEQ ID NO. 40 (zfNKBa) or any analogues and derivatives thereof.

As disclosed in Example 1, a second form of the NKB precursor, the “b” form has been identified by the inventors in both, zebrafish and Atlantic salmon. Thus, in other embodiments, the preprohormone peptide of the invention, may be translated from the tac3b cDNA and is therefore designated NKBb (Neurokinin Bb). In more specific embodiments, the preprohormone peptide of the invention may comprise an amino acid sequence as denoted by any one of SEQ ID NO: 50 and 64 and any analogues and derivatives thereof.

More specific embodiments related to the zebrafish preprohormone comprising the amino acid sequence according to SEQ ID NO. 50.

In yet another embodiment, the preprohormone of the invention comprises a first peptide fragment being the NKFb fragment that comprises the amino acid sequence as denoted by SEQ ID NO. 43.

In yet another embodiment, the preprohormone of the invention comprises a second peptide fragment that comprises an amino acid sequence as denoted by any one of SEQ ID NO. 41, (zfNKBb) or any analogues and derivatives thereof.

It should be appreciated that in all embodiments, the first neurokinin peptide fragment, defined as “NKFa” or “NKFb”, comprised in the neurokinin B preprohormone are derived from exon 3 of the neurokinin B gene.

Moreover, it should be generally noted that the second neurokinin peptide fragment defined as “NKBa”, which is comprised in the neurokinin B preprohormone, is derived from exon 5 of the neurokinin Ba gene and that the second neurokinin peptide fragment defined as “NKBb”, which is comprised in the neurokinin B preprohormone is derived from exons 3-5 of the neurokinin Bb gene.

The polypeptide Neurokinin B (NKB), which is a member of the tachykinin family of peptides, is a preprohormone (precursor) comprising two separate neurokinin polypeptide fragments, which, upon cleavage, mature to their active state. Generally, tachykinins precursors are cleaved at designated sites (normally Lys and/or Arg), by specific enzymes that include the prohormone convertases. The products are further post-translationally modified by the action of carboxypeptidases that remove the COOH-terminal dibasic residues, allowing the action of peptidylglycine α-amidating enzyme that converts the exposed COOH-terminal glycine residue into an amide. Such cleavage creates the active hormone or mature form of the peptide. Thus, the term “mature neurokinin polypeptide” or “active hormone peptide”, refers to any active NKB polypeptide that regulate reproduction in fish. The activity of the mature hormone peptides may be examined according to any method known to a person skilled in the art. Non limiting examples include following the signaling of the cognate receptor of the neurokinin polypeptide, namely, the neurokinin receptor (NKBR), as disclosed by Examples 8 and 12, or by following the elevation in the level of Leutininzing hormone (LH) and Follicle-stimulating hormone (FSH) upon administration of mature neurokinin polypeptides of the invention, as demonstrated in Examples 11, 13 and 14.

More specifically, as demonstrated by the inventors of the present invention, both first and second neurokinin peptide fragments, as defined above, were demonstrated to be active ligands of their cognate neurokinin receptor (nkbr also termed tac3r). The present invention encompasses neurokinin polypeptide or any analogues thereof, which comprise either both first and second neurokinin peptide fragments, or an active hormone peptide comprising only the first or the second neurokinin peptide fragment.

Thus, according to a second aspect, the invention relates to an isolated peptide comprising at least one of:

(a) a first peptide fragment comprises the amino acid sequence of:
X¹-X²-X³-X⁴-X⁵-X⁶-Asp⁷-X⁸-Phe⁹-Val¹⁰-X¹¹-Leu¹²-Met¹³, as denoted by SEQ ID NO. 96, Wherein:
X¹, is Tyr or any hydrophobic, a polar or positively charged amino acid selected from Phe, Ser, Lys and Gln;
X², is Asn or Ser or a hydrophobic, a polar, an acidic or positively charged amino acid selected from His, Thr, Asp, Arg, Lys and Ile;
X³, is Asp or a hydrophobic or a polar amino acid selected from Phe and Arg;
X⁴, is Ile or Leu or a polar or a hydrophobic amino acid selected from Asp, Tyr and Val;
X⁵, is Asp or an hydrophobic amino acid Met;
X⁶, is Tyr or acidic amino acid selected from Asp or His;
X⁸, is Ser or a polar or hydrophobic amino acid selected from Thr, Phe and Val;
X¹¹, is Gly or a polar amino acid, Ser;
X¹³, is Met or a hydrophobic amino acid Leu, and
(b) a second peptide fragment comprises the amino acid sequence of:
Glu¹-Met²-His³-Asp⁴-Ile⁵-Phe⁶-Val⁷-Gly⁸-Leu⁹-Met¹⁰ as denoted by SEQ ID NO. 40 and variants thereof. More specifically, the variants may comprise a substitution in at least one position selected from a group consisting of:
Glu¹ may be any one of Asn, Asp and Tyr; His³ may be substituted with any one of Asn and Asp; Asp⁴ may be Gln; Ile⁵ may be substituted with Val; Phe⁶ may be Leu; Val⁷ may be Ile; Gly⁸ may be substituted with Ala; and Met¹⁰ may be replaced with Leu.

It should be noted that the peptide of the invention regulates reproduction in fish.

According to one specific embodiment, the peptide of the invention comprises the first peptide fragment of the formula (the amino acid sequence of):

X¹-X²-X³-X⁴-X⁵-X⁶-Asp⁷-X⁸-Phe⁹-Val¹⁰-X¹¹-Leu¹²-Met¹³, as denoted by SEQ ID NO. 96, Wherein:
X¹, is Tyr or a hydrophobic, a polar or positively charged amino acid selected from Phe, Ser, Lys and Gln;
X², is Asn or Ser or a hydrophobic, a polar, an acidic or positively charged amino acid selected from His, Thr, Asp, Arg, Lys and Ile;
X³, is Asp or a hydrophobic or a polar amino acid selected from Phe and Arg;
X⁴, is Ile or Leu or a polar or a hydrophobic amino acid selected from Asp, Tyr and Val;
X⁵, is Asp or an hydrophobic amino acid Met;
X⁶, is Tyr or acidic amino acid selected from Asp or His;
X⁸, is Ser or a polar or hydrophobic amino acid selected from Thr, Phe and Val;
X¹¹, is Gly or a polar amino acid, Ser;
X¹³, is Met or a hydrophobic amino acid Leu.

In yet another specific embodiment, the peptide of the invention may be a tridecapeptide comprising an N terminal signature of Asp-Ile-Asp (DID), as indicated by the amino acid sequence of:

X¹-X²-Asp³-Ile⁴-Asp⁷-X⁸-Phe⁹-Val¹⁰-X¹¹-Leu¹²-Met¹³, as denoted by SEQ ID NO. 97, Wherein:
X¹, is Tyr or any hydrophobic, a polar or positively charged amino acid selected from Phe, Ser, Lys and Gln;
X², is Asn or Ser or a hydrophobic, a polar, an acidic or positively charged amino acid selected from His, Thr, Asp, Arg, Lys and Ile;
X³, is Asp or may be substituted with any one of Phe and Arg;
X⁴, is Ile or may be replaced with any one of Leu, Asp, Tyr and Val;
X⁵, is Asp or may be substituted with Met;
X⁶, is Tyr or acidic amino acid selected from Asp or His;
X⁸, is Ser or a polar or hydrophobic amino acid selected from Thr, Phe and Val;
X¹¹, is Gly or a polar amino acid, Ser;
X¹³, is Met or a hydrophobic amino acid Leu.

As shown by Example 1, mature hormone peptides were identified by the invention from different species of fish. Therefore, according to some specific embodiments, the peptide of the invention may be a peptide comprising an amino acid sequence as denoted by any one of SEQ ID NO. 42, 43, 53, 57, 61, 65, 68, 72, 76, 98, 100, 102, 103 and 105, or any analogs or derivatives thereof.

In yet more specific embodiments, the peptide of the invention may be a tridecapeptide comprising the amino acid sequence Tyr-Asn-Asp-Ile-Asp-Tyr-Asp-Ser-Phe-Val-Gly-Leu-Met-NH₂, as denoted by SEQ ID NO: 42, or Tyr-Asp-Asp-Ile-Asp-Tyr-Asp-Ser-Phe-Val-Gly-Leu-Met-NH₂, as denoted by SEQ ID NO:43, and any analogues and derivatives thereof.

Specific embodiments of the invention relate to the peptide of the invention being a tridecapeptide designated NKFa that consists of the amino acid sequence of Tyr-Asn-Asp-Ile-Asp-Tyr-Asp-Ser-Phe-Val-Gly-Leu-Met-NH₂, also in one letter code, YNDIDYDSFVGLM as denoted by SEQ ID NO:42, or any analogs and derivatives thereof.

According to some specific embodiments, the invention further provides an analog of the NKFa tridecapeptide of the invention, such analogue may comprise for example, the formula of Succ-Asp-Ser-Phe-N(Me)Val-Gly-Leu-Met-NH₂ as denoted by SEQ ID NO: 46.

According to other specific embodiments, the invention relate to the peptide of the invention being a trideca peptide designated NKFb that consists of the amino acid sequence of Tyr-Asp-Asp-Ile-Asp-Tyr-Asp-Ser-Phe-Val-Gly-Leu-Met-NH₂, as denoted by SEQ ID NO:43, and any analogues and derivatives thereof.

Still further, the NKF a and b tridecapeptides of the invention are zebrafish peptides. As shown by Example 1, the inventors identified NKF peptides in other species of fish.

Thus, according to another specific embodiment, the peptide of the invention may be a Pimephales promelas (fathead minnow) NKFa tridecapeptide having the amino acid sequence Tyr-Asn-Asp-Ile-Asp-Tyr-Asp-Ser-Phe-Val-Gly-Leu-Met-NH₂ (or in one letter code: YNDIDYDSFVGLM) as denoted by SEQ ID NO. 53, and any analogues and derivatives thereof.

In yet another specific embodiment, the peptide of the invention may be a Ictalurus punctatus (channel catfish) NKFa tridecapeptide having the amino acid sequence Tyr-His-Asp-Ile-Asp-Tyr-Asp-Ser-Phe-Val-Gly-Leu-Met-NH₂, (or in one letter code: YHDIDYDSFVGLM) as denoted by SEQ ID NO. 57, and any analogues and derivatives thereof.

In another specific embodiment, the peptide of the invention may be a Salmo salar (Atlantic salmon) NKFa tridecapeptide having the amino acid sequence Tyr-Asn-Asp-Leu-Asp-Tyr-Asp-Ser-Phe-Val-Gly-Leu-Met-NH₂ (or in one letter code: YNDLDYDSFVGLM) as denoted by SEQ ID NO. 61, and any analogues and derivatives thereof.

In another specific embodiment, the peptide of the invention may be a Salmo salar (Atlantic salmon) NKFb tridecapeptide having the amino acid sequence Tyr-Arg-Asp-Ile-His-Asp-Asp-Thr-Phe-Val-Gly-Leu-Met-NH₂ (or in one letter code: YRDIHDDTFVGLM) as denoted by SEQ ID NO. 65, and any analogues and derivatives thereof.

In another specific embodiment, the peptide of the invention may be a Dicentrarchus labrax (European seabass) NKFa tridecapeptide having the amino acid sequence Ser-Asp-Asp-Ile-Asp-Tyr-Asp-Thr-Phe-Val-Ser-Leu-Met-NH₂ (or in one letter code: SDDIDYDTFVSLM) as denoted by SEQ ID NO. 68, and any analogues and derivatives thereof.

In another specific embodiment, the peptide of the invention may be a tilapia Oreochromis niloticus NKFa tridecapeptide having the amino acid sequence Tyr-Asn-Asp-Leu-Asp-Tyr-Asp-Ser-Phe-Val-Gly-Leu-Met-NH₂ (or in one letter code: YNDLDYDSFVGLM) as denoted by SEQ ID NO. 72, and any analogues and derivatives thereof.

In another specific embodiment, the peptide of the invention may be a Oryzias latipes (Japanese medaka) NKFa tridecapeptide having the amino acid sequence Tyr-Thr-Asp-Leu-Asp-Tyr-Asp-Ser-Phe-Val-Gly-Leu-Met NH₂ (or in one letter code: YTDLDYDSFVGLM) as denoted by SEQ ID NO. 76, and any analogues and derivatives thereof.

In another specific embodiment, the peptide of the invention may be a Dissostichus mawsoni (Antarctic toothfish) NKFa tridecapeptide having the amino acid sequence Tyr-Ser-Asp-Leu-Asp-Tyr-Asp-Ser-Phe-Val-Gly-Leu-Met-NH₂ (or in one letter code: YSDLDYDSFVGLM) as denoted by SEQ ID NO. 98, and any analogues and derivatives thereof.

In another specific embodiment, the peptide of the invention may be a Sebastes rastrelliger (grass rockfish) NKFa tridecapeptide having the amino acid sequence Tyr-Ser-Asp-Leu-Asp-Tyr-Asp-Ser-Phe-Val-Gly-Leu-Met-NH₂ (or in one letter code: YSDLDYDSFVGLM) as denoted by SEQ ID NO. 100, and any analogues and derivatives thereof.

In yet another specific embodiment, the peptide of the invention may be a Gadus morhua (Atlantic cod) NKFa tridecapeptide having the amino acid sequence Ser-Ser-Asp-Leu-Asp-Tyr-Asp-Ser-Phe-Val-Gly-Leu-Met-NH₂ (or in one letter code: SSDLDYDSFVGLM) as denoted by SEQ ID NO. 102, and any analogues and derivatives thereof.

In another specific embodiment, the peptide of the invention may be an Arctic cod (Boreogadus saida) NKFa tridecapeptide having the amino acid sequence Phe-Ser-Asp-Leu-Asp-Tyr-Asp-Ser-Phe-Val-Gly-Leu-Met-NH₂ (or in one letter code: FSDLDYDSFVGLM) as denoted by SEQ ID NO. 103, and any analogues and derivatives thereof.

In another specific embodiment, the peptide of the invention may be an Osmerus mordax (rainbow smelt) NKFa tridecapeptide having the amino acid sequence Tyr-Ser-Asp-Val-Asp-Tyr-Asp-Ser-Phe-Val-Gly-Leu-Met-NH₂ (or in one letter code: YSDVDYDSFVGLM) as denoted by SEQ ID NO. 105, and any analogues and derivatives thereof.

According to other embodiments, the invention provides peptides comprising the second peptide fragment described by the invention. In these embodiments, the peptide of the invention may comprise the amino acid sequence of: Glu¹-Met²-His³-Asp⁴-Ile⁵-Phe⁶-Val⁷-Gly⁸-Leu⁹-Met¹⁰ as denoted by SEQ ID NO. 40 and variants thereof, said variants comprise a substitution in at least one position selected from a group consisting of: Glu¹ is any one of Asn, Asp and Tyr; His³ is any one of Asn and Asp; Asp⁴ is Gln; Ile⁵ is Val; Phe⁶ is Leu; Val⁷ is Ile; Gly⁸ is Ala; and Met¹⁰ is Leu.

More specific embodiments of peptide provided by the invention as comprising the second peptide fragment may be any peptide comprising the amino acid sequence as denoted by any one of SEQ ID NO. 40, 41, 54, 58, 62, 69, 73, 77, 93, 99, 101, 104 and 106, or any analogues and derivatives thereof.

More specific embodiments of the invention relate to a peptide designated NKBa. More specifically, the NKBa peptide of the invention comprises an amino acid sequence of Glu-Met-His-Asp-Ile-Phe-Val-Gly-Leu-Met-NH₂, also in one letter code EMHDIFVGLM, as denoted by SEQ ID NO:40 and any analogues and derivatives thereof.

In yet further embodiments, the invention provides analogs of the NKBa peptide of the invention, a non limiting example for an active analog is Succ-Asp-Ile-Phe-N(Me)Val-Gly-Leu-Met-NH₂ denoted by SEQ ID NO:44.

Still further embodiments of the invention provide a peptide designated NKBb. More particular embodiments of the NKBb of the invention may be a peptide having the amino acid sequence of Ser-Thr-Gly-Ile-Asn-Arg-Glu-Ala-His-Leu-Pro-Phe-Arg-Pro-Asn-Met-Asn-Asp-Ile-Phe-Val-Gly-Leu-Leu-NH₂, also in one letter code STGINREAHLPFRPNMNDIFVGLL, as denoted by SEQ ID NO: 41, and any analogues and derivatives thereof.

In certain embodiments the invention provides analogues of the NKBb peptide of the invention. One example for such analogue having the same biological activity, may be the peptide analog having the formula of Succ-Asp-Phe-N(Me)Val-Gly-Leu-Met-NH₂ denoted by SEQ ID NO:45.

In yet other certain embodiments, the invention provides analogues of the peptides described herein with the proviso that such analogues are not the analogues having the formula Succ-Asp-Phe-N(Me)Phe-Gly-Leu-Met-NH2, as also denoted by the SEQ ID NO:47.

Still further, the NKB a and b hormone peptides of the invention indicated above, are zebrafish peptides. As shown by Example 1, the inventors identified NKB peptides in other species of fish.

Thus, according to another specific embodiment, the peptide of the invention may be a Pimephales promelas (fathead minnow) NKBa peptide having the amino acid sequence Glu-Met-His-Asp-Ile-Phe-Val-Gly-Leu-Met-NH₂ (or in one letter code: EMHDIFVGLM) as denoted by SEQ ID NO. 54, and any analogues and derivatives thereof.

In yet another specific embodiment, the peptide of the invention may be a Ictalurus punctatus (channel catfish) NKBa peptide having the amino acid sequence Glu-Met-His-Asp-Ile-Phe-Val-Gly-Leu-Met-NH₂, (or in one letter code: EMHDIFVGLM) as denoted by SEQ ID NO. 58, and any analogues and derivatives thereof.

In another specific embodiment, the peptide of the invention may be a Salmo salar (Atlantic salmon) NKBa peptide having the amino acid sequence Glu-Met-Asp-Asp-Val-Phe-Val-Gly-Leu-Met-NH₂ (or in one letter code: EMDDVFVGLM) as denoted by SEQ ID NO. 62, and any analogues and derivatives thereof.

In another specific embodiment, the peptide of the invention may be a Salmo salar (Atlantic salmon) NKBb peptide having the amino acid sequence Asp-Met-Asp-Asp-Val-Phe-Val-Gly-Leu-Leu-NH₂ (or in one letter code: DMDDVFVGLL) as denoted by SEQ ID NO. 93, and any analogues and derivatives thereof.

In another specific embodiment, the peptide of the invention may be a Dicentrarchus labrax (European seabass) NKBa peptide having the amino acid sequence Tyr-Met-Asp-Gln-Ile-Leu-Ala-Ala-Leu-Leu-NH₂ (or in one letter code: YMDQILAALL) as denoted by SEQ ID NO. 69, and any analogues and derivatives thereof.

In another specific embodiment, the peptide of the invention may be a tilapia Oreochromis niloticus NKBa peptide having the amino acid sequence Glu-Met-Asp-Asp-Ile-Phe-Ile-Gly-Leu-Met-NH₂ (or in one letter code: EMDDIFIGLM) as denoted by SEQ ID NO. 73, and any analogues and derivatives thereof.

In another specific embodiment, the peptide of the invention may be a Oryzias latipes (Japanese medaka) NKBa peptide having the amino acid sequence Asp-Met-Asp-Asp-Ile-Phe-Val-Gly-Leu-Met-NH₂ (or in one letter code: DMDDIFVGLM) as denoted by SEQ ID NO. 77, and any analogues and derivatives thereof.

In another specific embodiment, the peptide of the invention may be a Dissostichus mawsoni (Antarctic toothfish) NKBa peptide having the amino acid sequence Glu-Met-Asn-Asp-Ile-Phe-Val-Glu-Leu-Met-NH₂ (or in one letter code: EMNDIFVGLM) as denoted by SEQ ID NO. 99, and any analogues and derivatives thereof.

In another specific embodiment, the peptide of the invention may be a Sebastes rastrelliger (grass rockfish) NKBa peptide having the amino acid sequence Glu-Met-His-Asp-Ile-Phe-Val-Glu-Leu-Met-NH₂ (or in one letter code: EMHDIFVGLM) as denoted by SEQ ID NO. 101, and any analogues and derivatives thereof.

In another specific embodiment, the peptide of the invention may be an Arctic cod (Boreogadus saida) NKBa peptide having the amino acid sequence Glu-Met-His-Asp-Ile-Phe-Val-Gly-Leu-Met-NH₂ (or in one letter code: EMHDIFVGLM) as denoted by SEQ ID NO. 104, and any analogues and derivatives thereof.

In another specific embodiment, the peptide of the invention may be an Osmerus mordax (rainbow smelt) NKBa peptide having the amino acid sequence Glu-Met-His-Asp-Ile-Phe-Val-Glu-Leu-Met-NH₂ (or in one letter code: EMHDIFVGLM) as denoted by SEQ ID NO. 106, and any analogues and derivatives thereof.

In certain embodiments, the peptide of the invention that comprises the second peptide fragment, has an Ile residue on position 5 (Ile). It must be understood that peptides such as the peptide of SEQ ID NO. 41 (NKBb) carry an equivalent Ile residue at position 17. However, although not having an Ile at position 5, the peptides EMDDVFVGLM as denoted by SEQ ID NO. 62, and the peptide DMDDVFVGLL as denoted by SEQ ID NO. 93, are also encompassed by the invention.

In certain embodiments, the invention provides an active NKB peptide having the structural properties as described herein above with the proviso that said peptide is not the peptide having the amino acid sequence DMHDFFVGLM-NH2, as denoted by SEQ ID NO:48.

In other embodiments, the peptide of the invention that comprises the second peptide fragment, has an Ile residue on position 5 (Ile). It must be understood that peptides such as the peptide of SEQ ID NO. 41 (NKBb) carry an equivalent Ile residue at position 17. However, although not having an Ile at position 5, the peptides EMDDVFVGLM as denoted by SEQ ID NO. 62, and the peptide DMDDVFVGLL as denoted by SEQ ID NO. 93, are also encompassed by the invention.

It must be understood that according to some embodiments, all active NKF or NKB peptides of the invention are indicated herein in their amidated form (NH₂), however the invention further encompasses also non-amidated forms of said peptides as also presented in the sequence listing.

It should be appreciated that in certain embodiments, the peptides of the invention are derived from the preprohormone peptide of the invention and therefore form the active hormone peptide described herein before.

As shown by Example 8, the active hormone peptides of the invention as well as analogues thereof, act as agonists of the NKB receptor (NKBR). Therefore, the active hormone peptides of the invention, as well as any active analogues thereof, specifically, peptides comprising the first or the second neurokinin peptide fragments according to the present invention, are also referred to as “ligands” or “agonists” that are able to bind and activate their cognate receptors, NKBR. In more specific embodiments, the analogue peptides of the invention, specifically, the analogues of SEQ ID NO. 44, 45 and 46, bind and activate the NKB receptor, and are therefore considered as agonists. In this connection, an agonist is a substance, specifically a peptide that binds to a receptor of a cell and triggers a response by that cell. Agonists often mimic the action of a naturally occurring substance that is a ligand.

As indicated above, the hormone peptides provided by the invention regulate reproduction in fish. The term “regulates” or “regulation” as used herein, refers to directing, governing, or controlling the physiology of reproduction in fish. The resulting biological outcome of such regulation, directing, governing, or controlling may be either positive, e.g. stimulation or enhancement of reproduction in fish, or negative, e.g. delaying the onset of or suppression of reproduction in fish.

The term “reproduction” as used herein, refers to the biological process by which new offspring individual organisms are produced from their parents. The present invention relates to sexual reproduction, which typically is generally defined as the creation of a new organism by combining the genetic material of two organisms.

In fish, as detailed above, sexual maturation, or “puberty” comprises the physiological and behavioral changes that occur during the transition from juvenile life into reproductive competence. One of the key events in the above transition is the onset of gonadotropin-releasing hormone (GnRH), and Follicle Stimulating Hormone (FSH) secretion.

In teleosts, one of three infraclasses of the ray-finned fishes, to which most living fishes belong, the pituitary receives a direct innervation by neurons sending projections to the vicinity of the pituitary gonadotrophs. Among the neurotransmitters and neuropeptides released by these nerve endings are GnRH and dopamine, acting as stimulatory and inhibitory factors (in many but not all fish) on the liberation of Luteinizng hormone (LH) and Follicle-stimulating hormone (FSH).

Thus, in all embodiments, the active hormone (mature) neurokinin polypeptides of the invention regulate reproduction in fish wherein said regulation may be positive or negative and wherein the active (mature) neurokinin polypeptide comprises at least one of said first or second neurokinin fragments.

As shown by Examples 8, 12, 13 and 14, the peptides of the invention lead to a significant increase in LH and FSH secretion in different fish species (zebrafish, Carp and Tilapia). Therefore, in certain embodiments, the invention provides active hormone peptides that stimulate and enhance different stages of the reproduction process in fish. More specifically, the phrase “active” as used herein in connection with the hormone peptides of the invention is used herein to indicate that the specific hormone peptide or any analogue thereof, exhibit a specific biological function that according to certain embodiments, is the regulation of different stages in fish reproduction, specifically, through the activation of NKB receptor.

In some embodiments, the active (mature) neurokinin polypeptide according to the invention comprises the first or the second neurokinin fragment as defined in the invention, or any derivative, analogue or homologue thereof, and in other embodiments, the precursor neurokinin polypeptide according to the invention comprises both the first and the second neurokinin fragment as defined in the invention, or any derivative, analogue or homologue thereof.

As shown by the following Examples, the active hormone peptides of the invention are particularly efficient in regulating different stages of reproduction, specifically, enhancing puberty in fish. More specifically, Examples 8 and 14 show that the hormone peptides of the invention, as well as their analogues, enhance secretion of LH and FSH in zebrafish, tilapia and in carp, therefore demonstrating the feasibility of the use of the peptides of the invention in regulating different stages of reproduction in fish. By way of non-limiting examples, fish species included in the present invention are zebrafish (Danio rerio), fathead minnow (Pimephales promelas), channel catfish (Ictalurus punctatus), Atlantic salmon (Salmo solar), Antarctic toothfish (Dissostichus mawsoni), grass rockfish (Sebastes rastrelliger), Atlantic cod (Gadus morhua), Arctic cod (Boreogadus saida), rainbow smelt (Osmerus mordax), Japanese medaka (Oryzias latipes), European seabass (Dicentrarchus labrax), rainbow trout (Oncorhynchus mykiss), brook trout (Salvelinus fontinalis), sablefish (Anoplopoma fimbria), copper rockfish (Sebastes caurinus), goldfish (Carassius auratus), three-spined stickleback (Gasterosteus aculeatus) and tilapia (Oreochromis niloticus).

The invention provides in the first and second aspects thereof, either the preprohormone peptides or the active hormone peptide derived from said precursor preprohormones. The term “polypeptide” as used herein refers to amino acid residues, connected by peptide bonds. A polypeptide sequence is generally reported from the N-terminal end containing free amino group to the C-terminal end containing free carboxyl group.

More specifically, “Amino acid sequence” or “peptide sequence” is the order in which amino acid residues connected by peptide bonds, lie in the chain in peptides and proteins. The sequence is generally reported from the N-terminal end containing free amino group to the C-terminal end containing amide Amino acid sequence is often called peptide, protein sequence if it represents the primary structure of a protein, however one must discern between the terms “Amino acid sequence” or “peptide sequence” and “protein”, since a protein is defined as an amino acid sequence folded into a specific three-dimensional configuration and that had typically undergone post-translational modifications, such as phosphorylation, acetylation, glycosylation, manosylation, amidation, carboxylation, sulfhydryl bond formation, cleavage and the like.

Amino acids, as used herein refer to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. “Amino acid analogs” refers to compounds that have the same fundamental chemical structure as a naturally occurring amino acid, i.e., an alpha carbon that is bound to a hydrogen, a carboxyl group, an amino group, and an R group, e.g., homoserine, norleucine, methionine sulfoxide, methionine methyl sulfonium. Such analogs have modified R groups or modified peptide backbones, but retain the same basic chemical structure as a naturally occurring amino acid. “Amino acid mimetics” refers to chemical compounds that have a structure that is different from the general chemical structure of an amino acid, but that functions in a manner similar to a naturally occurring amino acid Amino acids may be referred to herein by either their commonly known three letter symbols or by the one-letter symbols recommended by the IUPAC-IUB Biochemical Nomenclature Commission.

It should be noted that in addition to any of the preprohormone peptides of the invention and any active hormone or mature peptide derived therefrom, the invention further encompasses any derivatives, analogues, variants or homologues of any of the peptides or active hormones disclosed herein. The term “derivative” is used to define amino acid sequences (polypeptide), with any insertions, deletions, substitutions and modifications to the amino acid sequences (polypeptide) that do not alter the activity of the original polypeptides. By the term “derivative” it is also referred to homologues, variants and analogues thereof, as well as covalent modifications of a polypeptides made according to the present invention.

It should be noted that the polypeptides according to the invention can be produced synthetically, or by recombinant DNA technology. Methods for producing polypeptides peptides are well known in the art.

In some embodiments, derivatives include, but are not limited to, polypeptides that differ in one or more amino acids in their overall sequence from the polypeptides defined herein (either the preprohormones or the active hormone peptides of the invention), polypeptides that have deletions, substitutions, inversions or additions.

In some embodiments, derivatives refer to polypeptides, which differ from the polypeptides specifically defined in the present invention by insertions of amino acid residues. It should be appreciated that by the terms “insertions” or “deletions”, as used herein it is meant any addition or deletion, respectively, of amino acid residues to the polypeptides used by the invention, of between 1 to 50 amino acid residues, between 20 to 1 amino acid residues, and specifically, between 1 to 10 amino acid residues. More particularly, insertions or deletions may be of any one of 1, 2, 3, 4, 5, 6, 7, 8, 9, or 10 amino acids. It should be noted that the insertions or deletions encompassed by the invention may occur in any position of the modified peptide, as well as in any of the N′ or C′ termini thereof.

The peptides of the invention may all be positively charged, negatively charged or neutral. In addition, they may be in the form of a dimer, a multimer or in a constrained conformation, which can be attained by internal bridges, short-range cyclizations, extension or other chemical modifications.

The polypeptides of the invention can be coupled (conjugated) through any of their residues to another peptide or agent. For example, the polypeptides of the invention can be coupled through their N-terminus to a lauryl-cysteine (LC) residue and/or through their C-terminus to a cysteine (C) residue.

Further, the peptides may be extended at the N-terminus and/or C-terminus thereof with various identical or different amino acid residues. As an example for such extension, the peptide may be extended at the N-terminus and/or C-terminus thereof with identical or different amino acid residue/s, which may be naturally occurring or synthetic amino acid residue/s. An additional example for such an extension may be provided by peptides extended both at the N-terminus and/or C-terminus thereof with a cysteine residue. Naturally, such an extension may lead to a constrained conformation due to Cys-Cys cyclization resulting from the formation of a disulfide bond. Another example may be the incorporation of an N-terminal lysyl-palmitoyl tail, the lysine serving as linker and the palmitic acid as a hydrophobic anchor. In addition, the peptides may be extended by aromatic amino acid residue/s, which may be naturally occurring or synthetic amino acid residue/s, for example, a specific aromatic amino acid residue may be tryptophan. The peptides may be extended at the N-terminus and/or C-terminus thereof with various identical or different organic moieties, which are not naturally occurring or synthetic amino acids. As an example for such extension, the peptide may be extended at the N-terminus and/or C-terminus thereof with an N-acetyl group.

For every single peptide sequence defined by the invention and disclosed herein, this invention includes the corresponding retro-inverse sequence wherein the direction of the peptide chain has been inverted and wherein all the amino acids belong to the D-series.

The invention also encompasses any homologues of the polypeptides (either the active hormone or the preprohormone peptides) specifically defined by their amino acid sequence according to the invention. The term “homologues” is used to define amino acid sequences (polypeptide) which maintain a minimal homology to the amino acid sequences defined by the invention, e.g. preferably have at least about 65%, more preferably at least about 75%, even more preferably at least about 85%, most preferably at least about 95% overall sequence homology with the amino acid sequence of any of the polypeptide as structurally defined above, e.g. of a specified sequence, more specifically, an amino acid sequence of the polypeptides as denoted by any one of SEQ ID NO. 49, 52, 56, 60, 67, 71, 75, 79, 81, 83, 85, 89, 87, and 91 (the preprohormone sequences) and 42, 40, 41, 43, 53, 54, 57, 58, 61, 62, 65, 68, 69, 72, 73, 76, 77, 93, 98, 99, 100, 101, 102, 103, 104, 105 and 106 (the active hormone sequences).

More specifically, “Homology” with respect to a native polypeptide and its functional derivative is defined herein as the percentage of amino acid residues in the candidate sequence that are identical with the residues of a corresponding native polypeptide, after aligning the sequences and introducing gaps, if necessary, to achieve the maximum percent homology, and not considering any conservative substitutions as part of the sequence identity. Neither N- nor C-terminal extensions nor insertions or deletions shall be construed as reducing identity or homology. Methods and computer programs for the alignment are well known in the art.

In some embodiments, the present invention also encompasses polypeptides which are variants of, or analogues to, the polypeptides specifically defined in the invention by their amino acid sequence. With respect to amino acid sequences, one of skill will recognize that individual substitutions, deletions or additions to peptide, polypeptide, or protein sequence thereby altering, adding or deleting a single amino acid or a small percentage of amino acids in the encoded sequence is a “conservatively modified variant”, where the alteration results in the substitution of an amino acid with a chemically similar amino acid.

Conservative substitution tables providing functionally similar amino acids are well known in the art. Such conservatively modified variants are in addition to and do not exclude polymorphic variants, interspecies homologues, and alleles and analogous peptides of the invention.

For example, substitutions may be made wherein an aliphatic amino acid (G, A, I, L, or V) is substituted with another member of the group, or substitution such as the substitution of one polar residue for another, such as arginine for lysine, glutamic for aspartic acid, or glutamine for asparagine. Each of the following eight groups contains other exemplary amino acids that are conservative substitutions for one another:

### 1) Alanine (A), Glycine (G);

2) Aspartic acid (D), Glutamic acid (E);

### 3) Asparagine (N), Glutamine (Q);

### 4) Arginine (R), Lysine (K);

### 5) Isoleucine (I), Leucine (L), Methionine (M), Valine (V);

### 6) Phenylalanine (F), Tyrosine (Y), Tryptophan (W);

### 7) Serine (S), Threonine (T); and

### 8) Cysteine (C), Methionine (M)

More specifically, amino acid “substitutions” are the result of replacing one amino acid with another amino acid having similar structural and/or chemical properties, i.e., conservative amino acid replacements Amino acid substitutions may be made on the basis of similarity in polarity, charge, solubility, hydrophobicity, hydrophilicity, and/or the amphipathic nature of the residues involved. For example, nonpolar “hydrophobic” amino acids are selected from the group consisting of Valine (V), Isoleucine (I), Leucine (L), Methionine (M), Phenylalanine (F), Tryptophan (W), Cysteine (C), Alanine (A), Tyrosine (Y), Histidine (H), Threonine (T), Serine (S), Proline (P), Glycine (G), Arginine (R) and Lysine (K); “polar” amino acids are selected from the group consisting of Arginine (R), Lysine (K), Aspartic acid (D), Glutamic acid (E), Asparagine (N), Glutamine (Q); “positively charged” amino acids are selected form the group consisting of Arginine (R), Lysine (K) and Histidine (H) and wherein “acidic” amino acids are selected from the group consisting of Aspartic acid (D), Asparagine (N), Glutamic acid (E) and Glutamine (Q).

The derivatives of any of the polypeptides according to the present invention, e.g. of a specified sequence of any one of the polypeptides of SEQ ID NO. 49, 52, 56, 60, 67, 71, 75, 79, 81, 83, 85, 89, 87, and 91 (the preprohormone sequences) and 42, 40, 41, 43, 53, 54, 57, 58, 61, 62, 65, 68, 69, 72, 73, 76, 77, 93, 98, 99, 100, 101, 102, 103, 104, 105 and 106 (the active hormone sequences), may vary in their size and may comprise the full length polypeptide or any fragment thereof, comprising at least one of the neurokinin peptide fragments defined above. In some embodiments, the derivatives may include modified amino acid residues. Such modified amino acid residues include, but are not limited to the N-Me analog of the amino acid residue valine, in which the nitrogen atom is substituted with a methyl group.

In specific embodiments, analogues of the peptides defined by the present invention are generally prepared by omitting the N-terminal sequence up to Asp (D) and replacing the amino acid presiding Gly (G) (namely, Val) with its N-Me analog. For example, analogues of the polypeptides of the present invention are defined by, but not limited to, the amino acid sequences denoted SEQ ID NO. 44-46. As noted above, the peptides of the invention may be modified by omitting their N-terminal sequence, specifically, up to the Asp residue. However, it should be appreciated that the invention further encompasses the omission of about 1, 2, 3, 4, 5, 6, 7, 8, 9, 10, 11, 12, 13, 14, 15, 16, 17, 18, 19, 20 and more amino acid residues from both, the N′ and/or the C′ termini of the peptides. For example, three residues were omitted from NKBa to create the analogue of SEQ ID NO. 44, six residues were omitted from the N′ terminal sequence of NKF for preparing the analogue of SEQ ID NO. 46 and seventeen residues were omitted from NKBb FOR PREPARING THE ANALOGUE OF seq id n. 45.

As noted above, omission of the N terminal sequence was followed by replacement of Val with N-Me-Val. In yet more specific embodiments, it must be recognized that the invention encompasses analogues having at least one N-Me replacing any of the amino acid residues of the peptides of the invention at any position.

In certain embodiments the peptide compounds of the invention may comprise one or more amino acid residue surrogate. An “amino acid residue surrogate” as herein defined is an amino acid residue or peptide employed to produce mimetics of critical function domains of peptides.

Typically, peptide mimetics are designed and intended to fix and mimic the function of a dipeptide or tripeptide. For example, see the reverse-turn mimetics disclosed in U.S. Pat. Nos. 7,008,941, 6,943,157, 6,413,963, 6,184,223, 6,013,458 and 5,929,237, and U.S. Published Patent Application 2006/0084655, all describing various bicyclic ring structures asserted to mimic a dipeptide or tripeptide sequence. Other applications disclose a number of different small molecule compounds, again asserted to mimic a dipeptide or tripeptide sequence.

Examples of amino acid surrogate include, but are not limited to chemical modifications and derivatives of amino acids, stereoisomers and modifications of naturally occurring amino acids, non-protein amino acids, post-translationally modified amino acids, enzymatically modified amino acids, and the like. Examples also include dimers or multimers of peptides. An amino acid surrogate may also include any modification made in a side chain moiety of an amino acid. This thus includes the side chain moiety present in naturally occurring amino acids, side chain moieties in modified naturally occurring amino acids, such as glycosylated amino acids. It further includes side chain moieties in stereoisomers and modifications of naturally occurring protein amino acids, non-protein amino acids, post-translationally modified amino acids, enzymatically synthesized amino acids, derivatized amino acids, constructs or structures designed to mimic amino acids, and the like.

In some embodiments, the active hormone peptide according to the invention comprises an amino acid in comprising a derivative of the side chain moiety thereof. A “derivative of an amino acid side chain moiety”, as used herein, is a modification to or variation in any amino acid side chain moiety, including a modification to or variation in either a naturally occurring or unnatural amino acid side chain moiety, wherein the modification or variation includes: (a) adding one or more saturated or unsaturated carbon atoms to an existing alkyl, aryl, or aralkyl chain; (b) substituting a carbon in the side chain with another atom, preferably oxygen or nitrogen; (c) adding a terminal group to a carbon atom of the side chain, including methyl (—CH₃), methoxy (—OCH₃), nitro (—NO₂), hydroxyl (—OH), or cyano (—C═N); (d) for side chain moieties including a hydroxy, thio or amino groups, adding a suitable hydroxy, thio or amino protecting group; or (e) for side chain moieties including a ring structure, adding one or ring substituents, including hydroxyl, halogen, alkyl, or aryl groups attached directly or through an ether linkage. For amino groups, suitable amino protecting groups include, but are not limited to, Z, Fmoc, Boc, Pbf, Pmc and the like.

The peptide according to the invention may comprise an “N-Substituted Amino Acid”. An “N-substituted amino acid”, as described herein, includes any amino acid wherein an amino acid side chain moiety is covalently bonded to the backbone amino group, optionally where there are no substituents other than H in the α-carbon position. Sarcosine is an example of an N-substituted amino acid. By way of example, sarcosine can be referred to as an N-substituted amino acid derivative of Ala, in that the amino acid side chain moiety of sarcosine and Ala is the same, methyl.

In the course of a reaction of peptide synthesis, a nitrogen protecting group may be used. As used herein, “a nitrogen protecting group” means a group that replaces an amino hydrogen for the purpose of protecting against side reactions and degradation during a reaction sequence, for example, during peptide synthesis. Solid phase peptide synthesis involves a series of reaction cycles comprising coupling the carboxy group of an N-protected amino acid or surrogate with the amino group of the peptide substrate, followed by chemically cleaving the nitrogen protecting group so that the next amino-protected synthon may be coupled. Nitrogen protecting groups useful in the invention include nitrogen protecting groups well known in solid phase peptide synthesis, including, but not limited to, t-Boc (tert-butyloxycarbonyl), Fmoc (9-flourenylmethyloxycarbonyl), 2-chlorobenzyloxycarbonyl, allyloxycarbonyl (alloc), benzyloxycarbonyl, 2-(4-biphenylyl)propyl-2-oxycarbonyl (Bpoc), 1-adamantyloxycarbonyl, trityl (triphenylmethyl), and toluene sulphonyl.

In one embodiment, one amino acid surrogate may be employed in a peptide of the invention, two amino acid surrogates may be employed in a peptide of the invention, or more than two amino acid surrogates may be employed in a compound of the invention.

In another embodiment, there is provided a peptide including an amino acid surrogate wherein one or more peptide bonds between amino acid residues are substituted with a non-peptide bond.

In another embodiment of the invention, there is provided a peptide including at least one amino acid surrogate and a plurality of amino acid residues wherein the compound is a cyclic compound, cyclized by a bond between side chains of two amino acid residues, between an amino acid residue side chain and a group of an amino acid surrogate, between groups of two amino acid surrogate, between a terminal group of the compound and an amino acid residue side chain, or between a terminal group of the compound and a group of an amino acid surrogate.

In another embodiment, the peptide of the invention may include C-Terminus Capping Group. The term “C-terminus capping group” includes any terminal group attached through the terminal ring carbon atom or, if provided, terminal carboxyl group, of the C-terminus of a compound. The terminal ring carbon atom or, if provided, terminal carboxyl group, may form a part of a residue, or may form a part of an amino acid surrogate. In a preferred aspect, the C-terminus capping group forms a part of an amino acid surrogate which is at the C-terminus position of the compound. The C-terminus capping group includes, but is not limited to, —(CH₂)ₙ—OH, —(CH₂)ₙ—C(—O)—OH, —(CH₂)ₘ—OH, —(CH₂)ₙ—C(—O)—N(v₁)(v₂), —(CH₂)ₙ—C(—O)—(CH₂)ₘ—N(v₁)(v₂), —(CH₂)ₙ—O—(CH₂)ₘ—CH₃, —(CH₂)ₙ—C(—O)—NH—(CH₂)ₘ—CH₃, —(CH₂)ₙ—C(—O)—NH—(CH₂)ₘ—N(v₁)(v₂), —(CH₂)ₙ—C(—O)—N—((CH₂)ₘ—N(v₁)(v₂))₂, —(CH₂)ₙ—C(—O)—NH—CH(—C(—O)—OH)—(CH₂)ₘ—N(v₁)(v₂), —C(—O)—NH—(CH₂)ₘ—NH—C(—O)—CH(N(v₁)(v₂))((CH₂)ₘ—N(v₁)(v₂)), or —(CH₂)ₙ—C(—O)—NH—CH(—C(—O)—NH₂)—(CH₂)ₘ—N(v₁)(v₂), including all (R) or (S) configurations of the foregoing, where v₁ and v₂ are each independently H, a C₁ to C₁₇ linear or branched alkyl chain, m is 0 to 17 and n is 0 to 2; or any omega amino aliphatic, terminal aryl or aralkyl, including groups such as methyl, dimethyl, ethyl, propyl, isopropyl, butyl, isobutyl, pentyl, hexyl, allyl, cyclopropane methyl, hexanoyl, heptanoyl, acetyl, propionoyl, butanoyl, phenylacetyl, cyclohexylacetyl, naphthylacetyl, cinnamoyl, phenyl, benzyl, benzoyl, 12-Ado, 7′-amino heptanoyl, 6-Ahx, Amc or 8-Aoc, or any single natural or unnatural a-amino acid, beta-amino acid or a,a-disubstituted amino acid, including all (R) or (S) configurations of the foregoing, optionally in combination with any of the foregoing non-amino acid capping groups.

Still further embodiments relates to the peptides of the invention having an N-Terminus Capping Group. The term “N-terminus capping group” includes any terminal group attached through the terminal amine of the N-terminus of a compound. The terminal amine may form a part of a residue, or may form a part of an amino acid surrogate. In a preferred aspect, the N-terminus capping group forms a part of an amino acid surrogate which is at the N-terminus position of the compound. The N-terminus capping group includes, but is not limited to, any omega amino aliphatic, acyl group or terminal aryl or aralkyl including groups such as methyl, dimethyl, ethyl, propyl, isopropyl, butyl, isobutyl, pentyl, hexyl, allyl, cyclopropane methyl, hexanoyl, heptanoyl, acetyl, propionoyl, butanoyl, phenylacetyl, cyclohexylacetyl, naphthylacetyl, cinnamoyl, phenyl, benzyl, benzoyl, 12-Ado, 7′-amino heptanoyl, 6-Ahx, Amc or 8-Aoc, or alternatively an N-terminus capping group is —(CH₂)ₘ—NH(v₃), —(CH₂)ₘ—CH₃, —C(—O)—(CH₂)ₘ—CH₃, —C(—O)—(CH₂)ₘ—NH(v₃), —C(—O)—(CH₂)ₘ—C(—O)—OH, —C(—O)—(CH₂)ₘ—C(—O)-(v₄), —(CH₂)ₘ—C(—O)—OH, —(CH₂)ₘ—C(—O)-(v₄), C(—O)—(CH₂)ₘ—O(v₃), —(CH₂)ₘ—O(v₃), C(—O)—(CH₂)ₘ—S(v₃), or —(CH₂)ₘ—S(v₃), where v₃ is H or a C₁ to C₁₇ linear or branched alkyl chain, and v₄ is a C₁ to C₁₇ linear or branched alkyl chain and m is 0 to 17.

It should be appreciated that the invention further encompass any of the hormone peptides of the invention or any preprohormones referred herein, any serogates thereof, any salt, base, ester or amide thereof, any enantiomer, stereoisomer or disterioisomer thereof, or any combination or mixture thereof. Pharmaceutically acceptable salts include salts of acidic or basic groups present in compounds of the invention. Pharmaceutically acceptable acid addition salts include, but are not limited to, hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate and pamoate (i.e., 1,1′-methylene-bis-(2-hydroxy-3-naphthoate)) salts. Certain compounds of the invention can form pharmaceutically acceptable salts with various amino acids. Suitable base salts include, but are not limited to, aluminum, calcium, lithium, magnesium, potassium, sodium, zinc, and diethanolamine salts. For a review on pharmaceutically acceptable salts see BERGE ET AL., 66 J. PHARM. SCI. 1-19 (1977).

It should be noted that the present invention encompasses any fragment, derivative or analogue of any of the polypeptides of the invention, specifically, a fragment of any of the preprohormone peptides or the active hormone peptides of the invention that is a functional fragment. In certain embodiments, any of the polypeptides of the invention posses the ability to regulate reproduction in fish, specifically, through the activation of the NKB receptor. As used herein, the term “functional fragment”, “functional mutant”, “functional derivative” or “functional variant” refers to an amino acid sequence which possesses biological function or activity that is identical to the activity possessed by the original preprohormone or active hormone of the invention. Such activity may be identified through a defined functional assay. More specifically, the defined functional assay may be examined according to any method known to a person skilled in the art. Non limiting examples include following the signaling of the cognate receptor of the neurokinin polypeptide, namely, the neurokinin receptor (NKBR), as detailed herein below in the Examples section, or by following the elevation in the level of Leutininzing hormone (LH) and Follicle-stimulating hormone (FSH) upon administration of mature neurokinin polypeptides of the invention.

As indicated above, in certain embodiments, the invention provides isolated and purified hormone peptides, preprohormones thereof and isolated nucleic acid sequences encoding the same. As used herein, “isolated” or “substantially purified”, in the context of a peptide or nucleic acid molecule encoding said peptide, means the peptide or nucleic acid has been removed from its natural milieu or has been altered from its natural state. As such “isolated” does not necessarily reflect the extent to which the peptide or nucleic acid molecule has been purified. However, it will be understood that a peptide or nucleic acid molecule that has been purified to some degree is “isolated”. If the peptide or nucleic acid molecule does not exist in a natural milieu, i.e. it does not exist in nature, the molecule is “isolated” regardless of where it is present. Furthermore, the term “isolated” or “substantially purified”, when applied to a nucleic acid or protein, denotes that the nucleic acid or protein is essentially free of other cellular components with which it is associated in the natural state. It is preferably in a homogeneous state, although it can be in either a dry or aqueous solution. Purity and homogeneity are typically determined using analytical chemistry techniques such as polyacrylamide gel electrophoresis or high performance liquid chromatography. A protein which is the predominant species present in a preparation is substantially purified.

According to a second aspect, the invention provides an isolated polynucleotide sequence encoding a preprohormone peptide, that regulates reproduction in fish and comprises a first and a second peptide fragments. More specifically, the preprohormone peptides encoded by the polynucleotide sequence of the invention comprise both first and second fragments, (a) the first peptide fragment comprises the amino acid sequence of:

X¹-X²-X³-X⁴-X⁵-X⁶-Asp⁷-X⁸-Phe⁹-Val¹⁰-X¹¹-Leu¹²-Met¹³, as denoted by SEQ ID NO. 96, wherein:
X¹, is Tyr or any hydrophobic, a polar or positively charged amino acid selected from Phe, Ser, Lys and Gln;
X², is Asn or Ser or a hydrophobic, a polar, an acidic or positively charged amino acid selected from His, Thr, Asp, Arg, Lys and Ile;
X³, is Asp or a hydrophobic or a polar amino acid selected from Phe and Arg;
X⁴, is Ile or Leu or a polar or a hydrophobic amino acid selected from Asp, Tyr and Val;
X⁵, is Asp or an hydrophobic amino acid Met;
X⁶, is Tyr or acidic amino acid selected from Asp or His;
X⁸, is Ser or a polar or hydrophobic amino acid selected from Thr, Phe and Val;
X¹¹, is Gly or a polar amino acid, Ser;
X¹³, is Met or a hydrophobic amino acid Leu, and
(b) the second peptide fragment comprises the amino acid sequence of:
Glu¹-Met²-His³-Asp⁴-Ile⁵-Phe⁶-Val⁷-Gly⁸-Leu⁹-Met¹⁰ as denoted by SEQ ID NO. 40 and variants thereof. More specifically, the variants may comprise a substitution in at least one position selected from a group consisting of:
Glu¹ may be any one of Asn, Asp and Tyr; His³ may be substituted with any one of Asn and Asp; Asp⁴ may be Gln; Ile⁵ may be substituted with Val; Phe⁶ may be Leu; Val⁷ may be Ile; Gly⁸ may be substituted with Ala; and Met¹⁰ may be replaced with Leu.

As disclosed in the following Examples, the inventors have identified the tac3a and the tac3b nucleic acid sequences from various species of fish. According to certain specific embodiments, the polynucleotide molecule of the invention may comprise a nucleic acid sequence selected from the group consisting of SEQ ID NO: 4, 5, 51, 55, 59, 63, 66, 70, 74, 78, 80, 82, 84, 86, 88 and 90, or any fragment thereof.

As used herein, the term “polynucleotide” or “nucleic acid molecule”, or “nucleic acid sequence” refers to polymer of nucleotides, which may be either single- or double-stranded, which is a polynucleotide such as deoxyribonucleic acid (DNA), and, where appropriate, ribonucleic acid (RNA). The terms should also be understood to include, as equivalents, analogs of either RNA or DNA made from nucleotide analogs, and, as applicable to the embodiment being described, single-stranded (such as sense or antisense) and double-stranded polynucleotides. The term DNA used herein also encompasses cDNA, i.e. complementary or copy DNA produced from an RNA template by the action of reverse transcriptase (RNA-dependent DNA polymerase). The term is used to designate a single molecule, or a collection of molecules. Nucleic acids may be single stranded or double stranded, and may include coding regions and regions of various control elements, as described below.

More specific embodiments of the invention relate to a polynucleotide molecule comprising a nucleic acid sequence encoding an NKB a precursor, or preprohormone. Certain embodiments relate to the polynucleotide molecule being the zebrafish tac3a molecule as denoted by SEQ ID NO: 4, or any fragment thereof.

In yet another embodiment, the invention provides a polynucleotide molecule encodes the a fish NKBb precursor or preprohormone. More specifically, a nucleic acid sequence encoding the zebrafish NKBb, as denoted by the nucleic acid sequence of SEQ ID NO: 5 or any fragment thereof. Said polynucleotide molecule is designated by the inventors as zebrafish tac3b (zftac3b).

It should be noted that the invention further encompasses expression vectors comprising the polynucleotide sequences of the invention.

Expression vectors are typically self-replicating DNA or RNA constructs containing the desired gene or its fragments, and operably linked genetic control elements that are recognized in a suitable host cell and effect expression of the desired genes. These control elements are capable of effecting expression within a suitable host. Generally, the genetic control elements can include a prokaryotic promoter system or a eukaryotic promoter expression control system. This typically includes a transcriptional promoter, an optional operator to control the onset of transcription, transcription enhancers to elevate the level of RNA expression, a sequence that encodes a suitable ribosome binding site, RNA splice junctions, sequences that terminate transcription and translation and so forth. Expression vectors usually contain an origin of replication that allows the vector to replicate independently of the host cell.

A vector may additionally include appropriate restriction sites, antibiotic resistance or other markers for selection of vector-containing cells. Plasmids are the most commonly used form of vector but other forms of vectors which serve an equivalent function and which are, or become, known in the art are suitable for use herein. See, e.g., Pouwels et al., Cloning Vectors: a Laboratory Manual (1985 and supplements), Elsevier, N.Y.; and Rodriquez, et al. (eds.) Vectors: a Survey of Molecular Cloning Vectors and their Uses, Buttersworth, Boston, Mass. (1988), which are incorporated herein by reference.

Still further, the invention provides a host cell transformed or transfected with the expression vector according to the invention. These cells are designed to express any of the preprohormone or the active hormone peptides of the invention.

“Host cell” as used herein refers to cells which can be recombinantly transformed or transfected with naked DNA or expression vectors constructed using recombinant DNA techniques. A drug resistance or other selectable marker is intended in part to facilitate the selection of the transformants. Additionally, the presence of a selectable marker, such as drug resistance marker may be of use in keeping contaminating microorganisms from multiplying in the culture medium. Such a pure culture of the transformed host cell would be obtained by culturing the cells under conditions which require the induced phenotype for survival.

The host cells according to the invention are transformed or transfected with the polynucleotide according to the invention or with expression vector comprising thereof, to express the preprohormone peptides that are cleaved to produce the active hormone peptides of the invention, specifically, neurokinin polypeptides. The term “Transformation”, as used herein, refers to a process in which a cell's genotype is changed as a result of the cellular uptake of exogenous DNA or RNA, and, for example, the transformed cell expresses a recombinant form of the desired neurokinin polypeptide. The term “transfection” means the introduction of a nucleic acid, e.g., naked DNA or an expression vector, into a recipient cells by nucleic acid-mediated gene transfer.

The present invention demonstrate for the first time the effect of novel neurokinine hormone peptides on different stages of reproduction in fish. Therefore, according to another aspect, the invention relates to a composition regulating reproduction in fish. In certain embodiments, the composition of the invention comprises an effective amount of at least one of an isolated active hormone peptide derived from a preprohormone peptide, any analogues thereof, an isolated preprohormone peptide, that is the precursor peptide of the invention, any nucleic acid sequence encoding the preprohormone molecule of the invention, and any combinations thereof. In more specific embodiments, the preprohormone of the invention comprises a first and a second peptide fragments:

(a) the first peptide fragment comprises the amino acid sequence of:
X¹-X²-X³-X⁴-X⁵-X⁶-Asp⁷-X⁸-Phe⁹-Val¹⁰-X¹¹-Leu¹²-Met¹³, as denoted by SEQ ID NO. 96, Wherein:
X¹, is Tyr or any hydrophobic, a polar or positively charged amino acid selected from Phe, Ser, Lys and Gln;
X², is Asn or Ser or a hydrophobic, a polar, an acidic or positively charged amino acid selected from His, Thr, Asp, Arg, Lys and Ile;
X³, is Asp or a hydrophobic or a polar amino acid selected from Phe and Arg;
X⁴, is Ile or Leu or a polar or a hydrophobic amino acid selected from Asp, Tyr and Val;
X⁵, is Asp or an hydrophobic amino acid Met;
X⁶, is Tyr or acidic amino acid selected from Asp or His;
X⁸, is Ser or a polar or hydrophobic amino acid selected from Thr, Phe and Val;
X¹¹, is Gly or a polar amino acid, Ser;
X¹³, is Met or a hydrophobic amino acid Leu, and
(b) the second peptide fragment comprises the amino acid sequence of:
Glu¹-Met²-His³-Asp⁴-Ile⁵-Phe⁶-Val⁷-Gly⁸-Leu⁹-Met¹⁰ as denoted by SEQ ID NO. 40 and variants thereof. More specifically, the variants may comprise a substitution in at least one position selected from a group consisting of:
Glu¹ may be any one of Asn, Asp and Tyr; His³ may be substituted with any one of Asn and Asp; Asp⁴ may be Gln; Ile⁵ may be substituted with Val; Phe⁶ may be Leu; Val⁷ may be Ile; Gly⁸ may be substituted with Ala; and Met¹⁰ may be replaced with Leu.

It should be noted that in certain embodiments, the active hormone peptide derived from the preprohormone of the invention comprises at least one of the first or the second peptide fragments according to the invention.

In optional embodiments, the composition of the invention may further comprise a pharmaceutically acceptable carrier, excipient or diluent.

The compositions of the invention, as well as the methods described herein after, regulate reproduction in fish and therefore are intended for use for a fish subject. The term “fish” as used herein applies to a variety of more than 30,000 species of cold-blooded vertebrate animals found in the fresh and salt waters of the world. Remarkably, fish exhibit greater species diversity than any other group of vertebrates. Fish are gill-bearing aquatic craniate animals, including the living hagfish, lampreys, cartilaginous and bony fish, as well as various extinct related groups. Most fish are cold-blooded, allowing their body temperatures to vary as ambient temperatures change. More specific embodiment of the invention relate to the Actinopterygii class. Ray finned fish (Actinopterygii) constitute a class of subclass of the bony fish. The ray-finned fish are so called because they possess lepidotrichia or “fin rays”, their fins being webs of skin supported by bony or horny spines (“rays”). Actinopterygians are the dominant class of vertebrate, comprising nearly 96% of the 25,000 species of fish. They are ubiquitous throughout fresh water and marine environments, from the deep sea to the highest mountain streams and species can range in size from 8 millimetres (0.31 in), to the massive Ocean Sunfish, at 2,300 kilograms (5,100 lb).

In further specific embodiments, the invention provides hormone peptides, analogues and compositions thereof, as well as methods using the same for regulating reproduction in Teleosti. Teleostei is one of three infraclasses in class Actinopterygii, the ray-finned fishes. This diverse group includes 30,000 comprising virtually all the world's important sport and commercial fishes. Teleosts are distinguished primarily by the presence of a homocercal tail, a tail in which the upper and lower halves are about equal. The great abundance of some large species, such as tunas and halibuts and of smaller species, such as the various herrings, made teleosts extremely important to mankind as a food supply. Thus, in more specific embodiments, the compositions of the invention regulate reproduction in any fish of the Teleosti class and include any commercially farmed fish species, ornamental fish species either freshwater or saltwater species, including, without limitation, Carp, tilapia, masu salmon, Atlantic salmon, gilthead seabream (Sparus aurata), haddock, reedfish (Calamoichthys calabaricus), Sturgeons (Acipenseriformes), snook (Centropomus undecimalis), black sea bass (Centropristis striata), rainbow trout, monkfish, sole, perch, grouper, catfish, blue gill, yellow perch, white perch, sunfish, flounder, mahi mahi, striped bass, shad, pike, whitefish, swordfish, red snapper, baramundi, turbot, red drum, as well as ornamental species such as zebrafish.

More specific embodiments of the invention relate to any one of Carp, Tilapia and Salmon.

Thus, according to one specific embodiment, the composition, as well as the method of the invention described herein after, are particularly useful in increasing LH and FSH which are prerequisite for regulating reproduction in Carp. Carp are various species of freshwater fish of the family Cyprinidae (minnow and carp family), a very large group of fish native to Europe and Asia. Tribolodon is the only cyprinid genus which tolerates salt water, although there are several species which move into brackish water, but return to fresh water to spawn. All of the other cypriniformes live in continental waters and have a wide geographical range. The effect of the peptides of the invention on reproduction of Carp is demonstrated in Example 15.

Some consider all cyprinid fish carp, and the family Cyprinidae itself is often known as the carp family. In colloquial use, however, carp usually refers only to several larger cyprinid species such as Cyprinus carpio (common carp, one of the largest members of the minnow family), Carassius carassius (Crucian carp), Ctenopharyngodon idella (grass carp), Hypophthalmichthys molitrix (silver carp), and Hypophthalmichthys nobilis (bighead carp). Carp have long been an important food fish to humans, as well as popular ornamental fishes such as the various goldfish breeds and the domesticated common carp variety known as koi.

In yet another specific embodiment, the composition, as well as the method of the invention described herein after, are useful in regulating reproduction in Tilapia. Tilapia is the common name for nearly a hundred species of cichlid fish (part of the Oreochromis genus) from the tilapiine cichlid tribe. Tilapia inhabit a variety of fresh water habitats, including shallow streams, ponds, rivers and lakes, and are of increasing importance in aquaculture. Worldwide harvest of farmed tilapia has now surpassed 800,000 metric tons, and tilapia are second only to carps as the most widely farmed freshwater fish in the world. The effect of the peptides of the invention on reproduction of Tilapia is demonstrated in Examples 13-14.

In yet another specific embodiment, the composition, as well as the method of the invention described herein after, are useful in regulating reproduction in salmon. “Salmon” is the common name for several species of fish in the family Salmonidae, where several other fish in the same family are called “trout”. Salmon live along the coasts of both the North Atlantic (the migratory species Salmo salar) and Pacific Oceans (half a dozen species of the genus Oncorhynchus), and have also been introduced into the Great Lakes of North America Salmons are intensively produced in aquaculture in many parts of the world.

It should be appreciated that the composition of the invention as well as the methods described herein after are applicable for regulating reproduction in any other fish species, for example, tuna and kingfish.

As shown by Example 8, the novel active hormone peptides of the invention NKF and NKB a mediate activation of PKC and PKA through the fish Neurokinin receptor, NKBR, and therefore are considered as agonists of said receptor. Moreover, the inventors show that these fish hormone peptides also activate the human NKB receptor (huNKBR), acting as agonists. Therefore, although the compositions of the invention, as well as the active compounds comprised therein (the fish NKF, NKB peptides and analogues thereof) are intended for use in a fish subject, certain embodiments of the invention may also include a mammalian subject, specifically human. More specifically, the compositions of the invention may be applicable in inducing mammalian, and specifically, human NKBR mediated biological activity.

Thus, as used herein, the terms “subject in need thereof” or “subject” refer to an animal including fish, mammals, reptiles (specifically alligator), Xenopus (specifically, Xenopus tropicalis or Xenopus leavis) expressing the NKB receptor. Specifically the subject may be a mammal, such as for example rat, mice, dog, cat, guinea pig, primate and human or any organism for which administration of the active hormone peptides, specifically, NKF, NKB, analogues thereof or any composition or pharmaceutical composition of the invention is desired, in order to induce NKBR mediated biological activity. More specifically, said subject may be a mammalian subject, most specifically a human subject.

As indicated herein above, the compositions and methods of the invention are specifically applicable in regulating different stages of fish reproduction. More specifically, Fish species vary in their reproductive strategy and favored habitats for spawning and for early development of their newly hatched young. In addition, fish life cycles vary among species. In general, however, fish progress through the following life cycle stages: After passing through various stages of development, fertilized eggs are developed into fish. Importantly, most eggs do not survive to maturity, even under the finest conditions. Threats to eggs include changes in water temperature and oxygen levels, flooding or sedimentation, predators and disease. Developmental stages include the larva phase, where the term “larva” refers to a distinct juvenile form many animals undergo before metamorphosis into adults. Larval fish live off a yolk sac attached to their bodies. When the yolk sac is fully absorbed, the young fish are called “fry”. Fry are fish that ready to start feeding independently (i.e. they eat on their own). Fry undergo several more developmental stages, which vary by species, as they mature into adults. Young fish are generally considered fry during their first few months.

The term “juvenile fish” refers to the time period in which fish develop from fry into reproductively mature adults. This period varies among species. The term “adult fish” refers to fish that are able to reproduce. Fish maturation varies among species and individual fish with a shorter life span reaching maturity faster. For example, female round gobies mature in approximately one year and live for two to three years. Lake sturgeon live 80-150 years, and females mature when they are approximately 25 years old.

Spawning in fish refers to release of eggs by female fish into the water and to fertilization of eggs by releasing milt by male fish. Not all eggs are fertilized. Some fish spawn each year, some spawn several times at the same year, (or every one or more years) after reaching maturity, while others spawn only once and then die.

Therefore, the term “regulating reproduction in fish”, as used herein, refers to regulating any biological activity associated with delaying or advancing the onset of puberty. The term “puberty”, as herein defined, comprises the transition from an immature juvenile to a mature adult state of the reproductive system, when individual fish become capable of reproducing sexually for the first time.

Regulation of puberty is desirable since both early puberty and delayed puberty may be problematic, especially for farmed fish species. While early puberty has negative effects on growth performance, size, flesh composition, external appearance, behavior, health, welfare and survival, as well as of possible genetic impact on wild populations, late, or delayed puberty can also be a problem for broodstock management in some species. Furthermore, under farming conditions, some species completely fail to enter puberty [35].

Puberty or sexual maturation in fish is manifested in gonad growth, the production of fertile gametes, oocytes accumulating and rapid spermatogonial proliferation. In addition, pubertal fish are characterized with elevated plasma levels of several sex hormones, such as, for example, Estradiol (E2), Gonadotropin-releasing hormone (GnRH), Leutinizing hormone (LH) and Follicle Stimulating hormone (FSH) and in an elevated expression of key genes involved in reproduction (e.g. gnrh3, kiss2 and kiss1). Other parameters indicating puberty in fish are vitellogenesis (also known as yolk deposition, i.e. the process of yolk formation via nutrients being deposited in the oocyte). It has also been shown that in pubertal fish, an increase in cell number is observed in the ventral hypothalamus [13].

The physiological variations in the above parameters in fish before and towards puberty are known in the art and thus a skilled person will know how to identify pubertal fish based on measuring the above parameter. Methods of assessing the above physiological variations in fish before and towards puberty (i.e. during different stages of development) are well known in the art. For example, pubertal stage classification may be determined by histology of the gonads under light microscopy, as described by Biran et al. [4], based on the observation that pubertal fish gonads contain clear, well-developed oocytes and spermatozoa.

In addition, determining the expression levels of genes in fish may be performed by any method known in the art, for example mRNA of the above genes may be evaluated in fish brain during several different stages of development, by means of real-time PCR, as described herein below and plasma levels of the various hormones may be determined by any specific probes (such as, for example, using ELISA, as described in the invention).

According to certain and more specific embodiments, the composition of the invention may comprise an effective amount of at least one of: the isolated active hormone peptides derived from the preprohormones of the invention. Such active hormone peptide comprise at least one of the first or the second peptide fragments according to the invention. Non limiting examples for such active hormone peptides are the peptides as denoted by any one of SEQ ID NO. 42, 40, 41, 43, 53, 54, 57, 58, 61, 62, 65, 68, 69, 72, 73, 76, 77, 93, 98, 99, 100, 101, 102, 103, 104, 105 and 106, any analogues, derivatives or variants thereof. In yet another embodiment, the composition of the invention may comprise analogs of the active hormone peptides of the invention, specifically, as denoted by any one of SEQ ID NO. 44, 46 and 45. Alternatively, the composition of the invention may comprise as an active ingredient any of the isolated preprohormone (or precursor peptide) as denoted by any one of SEQ ID NO. 49, 50, 52, 56, 60, 64, 67, 71, 75, 79, 81, 83, 85, 89, 87, and 91 and any homologs, analogs and derivatives thereof. In other embodiments, the composition of the invention may comprise as an active ingredient any nucleic acid sequence encoding said preprohormone, as denoted by any one of SEQ ID NO. 4, 5, 51, 55, 59, 63, 66, 70, 74, 78, 80, 82, 84, 86, 88 and 90, and any combinations thereof.

It should be appreciated that the invention further encompasses compositions comprising as an active ingredient any variant, homolog or derivative of any of the peptides or the nucleic acid sequences described by the invention.

According to one specific embodiment, the composition of the invention may comprise as an active ingredient an effective amount of any of the active hormone peptides of the invention. As noted herein before, each of the active hormone peptides of the invention comprise at least one of the first or the second peptide fragments defined herein before.

According to another specific embodiment, the composition of the invention may comprise at least one active hormone peptide that comprises the first peptide fragment according to the invention. More specifically, the composition of the invention may comprise an effective amount of at least one trideca NKF active hormone peptides of the invention, as denoted by any one of SEQ ID NO. 42, 43, 53, 57, 61, 65, 68, 72, 76, 98, 100, 102, 103 and 105, any combinations thereof or any analogues, variants or derivatives thereof. Certain non-limiting example for such analogs may be the analog as denoted by SEQ ID NO. 46.

In yet more specific embodiments, the composition of the invention may comprise as an active ingredient an effective amount of an active hormone peptide of the invention designated NKFa. In further specific embodiments, this peptide is a trideca peptide that comprises the amino acid sequence of Tyr-Asn-Asp-Ile-Asp-Tyr-Asp-Ser-Phe-Val-Gly-Leu-Met-NH₂, as denoted by SEQ ID NO:42, or any analogs and derivatives thereof.

In another specific embodiment, the composition of the invention may comprise as an active ingredient, an analog of the NKFa trideca active hormone peptide of the invention. A specific and non limiting example for such trideca peptide analog is the Succ-Asp-Ser-Phe-N(Me)Val-Gly-Leu-Met-NH₂ analog as denoted by SEQ ID NO:46.

In yet another specific embodiment, the composition of the invention may comprise as an active ingredient, the active hormone peptide designated NKFb. In certain embodiments, such active hormone peptide is a trideca peptide comprising the amino acid sequence as denoted by SEQ ID NO. 43.

In yet another embodiment, the composition of the invention comprises an effective amount of at least one active hormone peptide that comprises the second peptide fragment as defined by the invention. Such active hormone peptides are designated by the invention as NKB peptides and may comprise the amino acid sequence of any one of SEQ ID NO. 40, 41, 54, 58, 62, 69, 73, 77, 93, 99, 101, 104 and 106.

In yet another specific embodiment, the composition of the invention may comprise as an active ingredient, an NKB active hormone peptide, specifically, the NKBa that comprises the amino acid sequence of Glu-Met-His-Asp-Ile-Phe-Val-Gly-Leu-Met NH₂, as denoted by SEQ ID NO:40, and any analogues and derivatives thereof.

In another particular embodiment, the composition of the invention may comprise an analog of the NKBa peptide of SEQ ID NO. 40. A non-limiting example for such peptide analog is the Succ-Asp-Ile-Phe-N(Me)Val-Gly-Leu-Met-NH₂, as denoted by SEQ ID NO: 44.

In yet another specific embodiment, the composition of the invention may comprise as an active ingredient at least one active hormone peptide designated NKBb. In one specific embodiment, this peptide comprises the amino acid sequence of Ser-Thr-Gly-Ile-Asn-Arg-Glu-Ala-His-Leu-Pro-Phe-Arg-Pro-Asn-Met-Asn-Asp-Ile-Phe-Val-Gly-Leu-Leu-NH₂, as denoted by SEQ ID NO:41, or any analogues and derivatives thereof.

In another specific embodiment, the composition of the invention may comprise as active ingredient, an analogue of the active hormone peptide NKBb of the invention. A non-limiting example for such analogue is Succ-Asp-Phe-N(Me)Val-Gly-Leu-Met-NH₂ denoted by SEQ ID NO:45.

It should be appreciated that the compositions disclosed herein may comprise as an active ingredient any combination of the active hormone peptides of the invention or of any analogues thereof. For example, a combination of at least one NKF active hormone peptide or analogues thereof with at least one NKB hormone peptide or any analogues thereof.

In yet another embodiment, the compositions of the invention may comprise a combination of at least one of the NKF and the NKB active peptides of the invention with another regulator of fish reproduction, for example, GnRHa or KISS.

According to specific embodiments, the composition of the invention is particularly applicable for regulating reproduction in fish, specifically, any stage of the reproduction process in fish. The stages of reproduction in fish, referred to herein may include growth of gonads (which are the organs that produce gametes; in males the gonads are the testes and in female the gonads are the ovaries), production of gametes, and reproductive behaviour.

In all embodiments, the composition according to the invention is for regulating reproduction in fish. The term “regulating reproduction in fish”, as used herein, refers to at least one of the following non-limiting biological activities: advancing the onset of puberty, regulating the timing and amount of ovulation and spawning, synchronization or stimulation of reproduction, enhancing the development of gammets, enhancing vitellogenesis, induction of Gonadotropin-releasing hormone (GnRH), increasing the level of Luteinizing-hormone (LH), Follicle-stimulating hormone (FSH) or of any other hypothalamic neuropeptide or neurohormore, induction of the Kisspeptine pathway, induction of oocyte maturation and activation of the PKC and PKA pathways.

Thus, the term “advancing the onset of puberty”, as herein defined, is used in the broadest sense and relates to advancing the age of onset of any of the above noted biological activities, for example, but not limited to, advancing the age of onset of gonad growth, advancing the age of onset of production of fertile gametes, or advancing the age of onset of oocytes accumulating in fish, relative to the age at which the above referred to biological activities occur in the absence of the composition of the invention.

In some embodiments, the composition according to the invention is for regulating the timing and amount of ovulation and spawning. The term “regulating the timing and amount of ovulation and spawning” as herein defined, relates to any delay or advance of the time at which fish ovulate (the process by which a mature ovarian follicle ruptures and discharges oocytes) or spawn (the process of releasing the eggs and sperm, usually into water). These physiological phenomena may be monitored by any method known to a person skilled in the art, for example, by microscopic methods or manual methods.

In some embodiments, the composition according to the invention is for synchronization or stimulation of reproduction. The term “synchronization of reproduction”, as herein defined, relates to determining or coordinating the onset of reproduction, which is the biological process by which new off-springs individual organism are produced from their parents (e.g. fish spawning). The onset and the amount of fish spawning may be monitored by methods well known in the art. The term “stimulation of reproduction”, as herein defined, refers to inducing, exciting or increasing the amount of reproduction as herein defined. More specifically, the term “synchronization” as referred to herein refers to induction of the physiological phenomena as detailed herein below in a controlled fashion, i.e., such that all fish in an aquaculture will undergo the developmental stages towards puberty in a cynchronized, controlled manner, upon administration of the compound or composition of the invention. Thus, using compound or composition of the invention, synchronization of the following life cycle developmental stages in fish may be achieved: (1) spawning, i.e. induction of spawning to occur at controlled timing by administering the fish with the compound or composition of the invention; (2) frying, i.e. induction of the passage from the development stage of larva to the development stage of a fry and thus enhancing maturation of fish, by immersion of fish culture in a medium contacting the compound or composition of the invention; (3) induction of the passage from the development stage of a fry to the development stage of a juvenile fish; (4) maturation, by feeding, immersing or administrating in any other route the compound or composition of the invention to juvenile fish.

In further embodiments, the composition according to the invention is for enhancing the development of gametes. A “gamete”, as herein defined, is a cell that fuses with another cell during fertilization in organisms that reproduce sexually. In species that produce two morphologically distinct types of gametes, and in which each individual produces only one type, a female produces the larger type of gamete, called an “oocyte” and a male produces the smaller type of gamete, called a “sperm”. Determining the developmental stage of gametes may be performed by any method known to a person skilled in the field of invention, for example, by monitoring the dimensions and morphology of the oocyte and sperm, using microscopic methods.

In yet further embodiments, the composition according to the invention is for enhancing vitellogenesis. Vitellogenesis, as defined herein above, may be monitored by any method known to a person skilled in the art, for example, by using specific antibodies or by monitoring the levels of plasma calcium and estradiol concentrations.

In some embodiments, the composition according to the invention may be used for induction of oocyte maturation. The term “induction of oocyte maturation”, as used herein, refers to stimulating, or advancing the onset of oocyte maturation. Methods for monitoring oocyte maturation are known in the art, for example, oocyte maturation may be determined for example, by histological assessment.

In some embodiments, the composition according to the invention may be particularly applicable for induction of Gonadotropin-releasing hormone (GnRH), increasing the level of Luteinizing-hormone (LH), Follicle-stimulating hormone (FSH) or any other hypothalamic neuropeptide or neurohormone (for example, kisspeptin1, kisspeptin2, oxcytocin, Neuropeptide Y, melanocyte-stimulating hormones). Methods for determining the plasma levels of hormones are known in the art. For example, the level of GnRH, LH or FSH, or any other hormones or neurohormore may be determined by ELISA test, using specific antibodies or by other methods.

Using the specific reporters SRE-Luc and CRE-Luc as described by Example 8, the inventors demonstrate the activation of PKC/Ca²⁺ and PKA/cAMP signal transduction pathways by the active hormone peptides of the invention. Thus, in some embodiments, the composition according to the invention may be applicable for the activation of the PKC and/or PKA pathways in a subject in need thereof.

In some further embodiments, the composition according to the invention may be used for induction of the Kisspeptin pathway. As recited herein above, Kisspeptin, which is a neuropeptide encoded by the Kiss1 gene, is a potent secretagogue for gonadotropin-releasing hormone (GnRH) in mammals and may be a critical component of pubertal maturation based on several observations.

As indicated above, the composition of the invention are specifically intended for regulating reproduction in fish, as used herein, regulation refers either to advancing, enhancing, elevating, increasing and inducing any biological activity associated with reproduction in fish, or alternatively, delaying, attenuating, decreasing, reducing or inhibiting, any of said reproduction associated parameters indicated herein above. More specific embodiments of the invention relate to composition for advancing and enhancing different stages of fish reproduction. Specifically, as reflected by any of the above mentioned reproduction associated parameters, or any combinations thereof.

In some specific embodiments, the invention provides compositions as herein described, wherein said advancing or advance results in a change, alteration, modification with respect to fish prior to the treatment or administration, or fish that were not administered with the composition of the invention, of at least about 1%-100%, about 5%-95%, about 10%-90%, about 15%-85%, about 20%-80%, about 25%-75%, about 30%-70%, about 35%-65%, about 40%-60% or about 45%-55%.

More specifically, said change, alteration, modification, elevation or an increase may also be by at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or about 100%.

Moreover, with regards to the above, it is to be understood that, where provided, percentage values such as, for example, 10%, 50%, 100%, end even more, etc., are interchangeable with “fold change” values, i.e., 0.1, 0.5, 1.2, 5, etc.

The composition of the invention comprises an effective amount of at least one of the isolated active hormone peptides, and analogues thereof or the preprohormone peptide of the invention, as herein defined. The term “effective amount”, as herein defined may be determined by such considerations as known in the art. The amount must be effective to achieve the desired effect as described below, depending, inter alia, on the type and weight of the subject concerned. The effective amount is typically determined in appropriately designed trials (dose range studies) and the person versed in the art will know how to properly conduct such trials in order to determine the effective amount. As generally known, an effective amount depends on a variety of factors including the affinity of the ligand to the receptor, its distribution profile within the body, a variety of pharmacological parameters such as half life in the body, on undesired side effects, if any, etc.

In certain embodiments, the composition of the invention comprises an effective amount of at least one of an isolated active hormone peptides of the invention, any analogues thereof or any of the preprohormone peptides of the invention. In specific embodiments where the active ingredient is any of the active hormone peptides of the invention, specifically, any one of the NKF peptides or the NKB peptides and any analogues thereof, an effective amount of such active ingredient may range between about 0.1 to about 100 pmol/g body weight (BW), between about 0.5 to about 90 pmol/g BW, between about 1 to about 95 pmol/gBW, between about 5 to about 80 pmol/gBW, between about 10 to about 70 pmol/gBW, between about 15 to about 60 pmol/gBW, between about 20 to about 50 pmol/gBW, between about 20 to about 40 pmol/gBW, between about 20 to about 30 pmol/gBW or between about 20 to about 25 pmol/gBW.

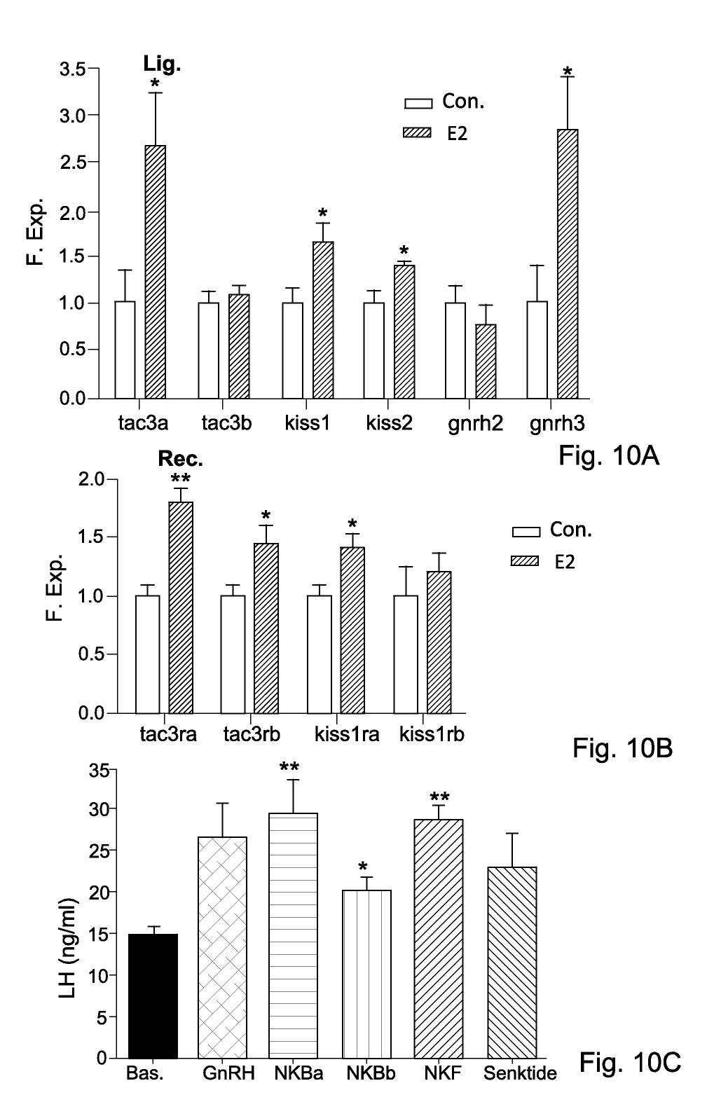

FIG. 10C demonstrates the feasibility of using an effective amount of about 20 pmol/gBW of any of the active hormone peptides of the invention, specifically, zfNKBa, zfNKBb or zfNKF (as also denoted by SEQ ID NO. 40, 41 and 42, respectively) to mature zebrafish, inducing thereby, a significant increase in LH secretion. Thus, specific and non limiting examples relate to compositions comprising an effective amount of between about 10 to about 40 pmol/gBW, specifically, between about 15 to about 30 pmol/gBW, and more specifically, between about 20 to about 25 pmol/gBW of any of the NKF, specifically, NKFa, NKB, specifically, NKBa, and any analogues thereof, specifically, any of the analogues of SEQ ID NO. 46, 44 and 45.

In still further specific embodiments, the composition of the invention may comprise an effective amount of at least one of the active hormone peptides of the invention, specifically, any one of the NKF peptides or the NKB peptides and any analogues thereof, that may range between about 0.05 to about 500 μg/Kg body weight (BW), between about 0.1 to about 400 μg/Kg BW, between about 0.2 to about 300 μg/Kg, BW between about 0.3 to about 300 μg/Kg BW, between about 0.4 to about 200 μg/Kg BW, between about 0.5 to about 100 μg/Kg BW, between about 0.5 to about 10 μg/Kg BW, and between about 0.5 to about 5 μg/Kg BW. More specifically, about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100 μg/Kg BW and more.

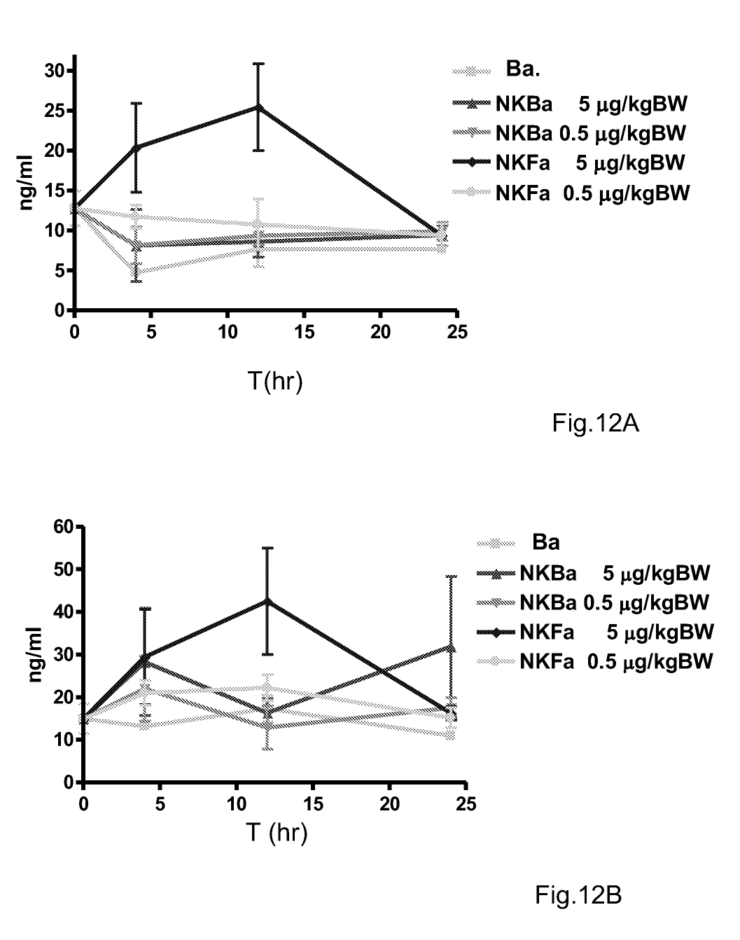

As shown by FIG. 12, NKF and NKBa analogues were injected to Juvenile Tilapia at 0.5 or 5 μg/Kg BW, inducing thereby a significant increase in LH and FSH secretion.

A significant increase has been exhibited by the NKF analogue at 5 μg/Kg BW. Thus, a specific and non limiting example relate to compositions comprising an effective amount of between about 0.1 to about 10 μg/Kg BW, between about 0.2 to about 9 μg/Kg BW, between about 0.3 to about 8 μg/Kg BW, between about 0.4 to about 7 μg/Kg BW, between about 0.5 to about 6 μg/Kg BW and between about 0.5 to about 5 μg/Kg BW. More specifically, an amount of about 0.5 or 5 μg/Kg BW, specifically about 5 μg/Kg BW.

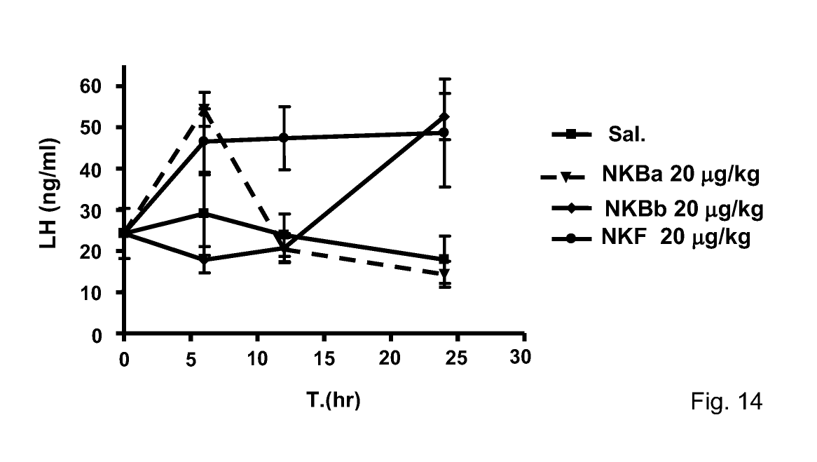

FIG. 14 demonstrates the injection of an amount of about 20 μg/Kg BW of any one of NKF, NKBa and NKBb analogues (SEQ ID NO. 46, 44 and 45, respectively) to mature female carp before spawning. All the analogues showed a significant elevation in LH levels. Thus, a specific and non limiting example relate to compositions comprising an effective amount of between about 1 to about 50 μg/Kg BW, between about 5 to about 40 μg/Kg BW, between about 10 to about 30 μg/Kg BW, between about 15 to about 25 μg/Kg BW, between about 20 to about 25 μg/Kg BW. More specifically, an amount of about 20 μg/Kg BW of any of the analogue peptides of the invention, specifically, any of the analogues of SEQ ID NO. 46, 44 and 45.

As noted above, any of the compositions of the invention may comprise pharmaceutically acceptable carriers, vehicles, adjuvants, excipients, or diluents. As used herein pharmaceutically acceptable carriers, vehicles, adjuvants, excipients, or diluents, are well-known to those skilled in the art and are readily available to the public. It is preferred that the pharmaceutically acceptable carrier be one which is chemically inert to the active compounds and one which has no detrimental side effects or toxicity under the conditions of use.

The choice of a carrier will be determined in part by the particular active agent, as well as by the particular method used to administer the composition. The carrier can be a solvent or a dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants.

Each carrier should be both pharmaceutically and physiologically acceptable in the sense of being compatible with the other ingredients and not injurious to the subject, specifically, fish. Formulations include those suitable for immersion, oral, parenteral (including subcutaneous, intramuscular, intravenous, intraperitoneal, implantation for slow release and intradermal) administration. The formulations may conveniently be presented in unit dosage form and may be prepared by any methods well known in the art of pharmacy. The nature, availability and sources, and the administration of all such compounds including the effective amounts necessary to produce desirable effects in a subject are well known in the art and need not be further described herein.

The compositions according to the present invention may be administered by any suitable route including injection, implantation, immersing the embryonic or juvenile fish in a bath of an effective substance or through a feed product. For example, and not by way of limitation, the active ingredient, specifically, any of the active hormone peptides of the invention and analogues thereof may be directly injected intramuscularly into the fish. In one embodiment, the active hormone peptide may be combined with a polymer based carrier matrix into a sustained release delivery system.

The term “sustained release” is understood to mean a gradual release of the active compound in a controlled manner A suitable carrier having such sustained release properties may be chosen on the basis of its gradual release properties in a solution designed to resemble a fish's plasma, such as a ringer solution, other physiological saline solutions, fish serum, etc.

The compositions of the present invention, if delivered in a solid form, may be prepared in any suitable form such as pellets, discs, rods or microspheres. These may be administered to the fish larvae either by implantation of a composition unit (in the form of a pellet, disc or rod) or by injection, intramuscular, subcutaneous or intraperitoneal (in the form of a suspension of mini-rods or micro-spheres). Alternatively, a composition in a solid form may be administered by feed as an oral composition.

In case of implantable composition, the size of such composition in accordance with the present invention will be determined both by the size of the fish in which implantation thereof is intended, i.e. it should not be too big, and by practical limitations, i.e. the implantable composition should not be too small so as to render it difficult for manipulation. Thus, for example, a disc having a diameter of about 1 to 10 mm and a thickness of about 0.01 to 2 mm has been found in the art to be suitable for implantation in many fish such as the sea bream, sea bass and trout.

The composition may be administered to the fish either by subcutaneous or intraperitoneal implantation (for injectable micro-rods or spheres). For subcutaneous implantation a small incision are made through the fish's skin at a suitable place and after separating the skin from the underlying muscles, e.g., by the use of forceps, the implantation and incision is made through the skin and muscle of the peritoneal cavity and the implant is inserted through the incision and placed in the peritoneum. The incision in each case is made as small as practicably possible and there is usually no need for post implantational stitching.

Injectable compositions in accordance with the invention in the form of mini-rods or microspheres should be sufficiently small to pass through a syringe. Injectable compositions will be suspended in an injectable solution, such as saline or various buffers, prior to injection, and thereafter the suspension is injected into a suitable muscle of the fish or into the peritoneal cavity.

It must be understood that all ranges and description of effective amounts, biological activity and effect on different stages of fish reproduction disclosed herein as part of the composition aspect, are also applicable to any of the other aspects of the invention, specifically, for any of the methods and uses of the invention.

According to a fifth aspect, the invention provides a method for regulating reproduction in fish. In certain embodiments, the method of the invention comprises the step of administering to a treated fish an effective amount of at least one of: an isolated active hormone peptide, specifically the peptide derived from the preprohormone of the invention, any analogues thereof, any of the preprohormone peptides (precursor for the active peptide of the invention), any nucleic acid sequence encoding said preprohormone, any combinations thereof and any composition comprising the same. It should be noted that the preprohormone (precursor) of the invention comprises a first and a second peptide fragments. More specifically, (a) the first peptide fragment comprises the amino acid sequence of:

X¹-X²-X³-X⁴-X⁵-X⁶-Asp⁷-X⁸-Phe⁹-Val¹⁰-X¹¹-Leu¹²-Met¹³, as denoted by SEQ ID NO. 96, Wherein:
X¹, is Tyr or any hydrophobic, a polar or positively charged amino acid selected from Phe, Ser, Lys and Gln;
X², is Asn or Ser or a hydrophobic, a polar, an acidic or positively charged amino acid selected from His, Thr, Asp, Arg, Lys and Ile;
X³, is Asp or a hydrophobic or a polar amino acid selected from Phe and Arg;
X⁴, is Ile or Leu or a polar or a hydrophobic amino acid selected from Asp, Tyr and Val;
X⁵, is Asp or an hydrophobic amino acid Met;
X⁶, is Tyr or acidic amino acid selected from Asp or His;
X⁸, is Ser or a polar or hydrophobic amino acid selected from Thr, Phe and Val;
X¹¹, is Gly or a polar amino acid, Ser;
X¹³, is Met or a hydrophobic amino acid Leu, and
(b) the second peptide fragment comprises the amino acid sequence of:
Glu¹-Met^{e}-His³-Asp⁴-Ile⁵-Phe⁶-Val⁷-Gly⁸-Leu⁹-Met¹⁰ as denoted by SEQ ID NO. 40 and variants thereof. More specifically, the variants may comprise a substitution in at least one position selected from a group consisting of:
Glu¹ may be any one of Asn, Asp and Tyr; His³ may be substituted with any one of Asn and Asp; Asp⁴ may be Gln; Ile⁵ may be substituted with Val; Phe⁶ may be Leu; Val⁷ may be Ile; Gly⁸ may be substituted with Ala; and Met¹⁰ may be replaced with Leu.

It should be noted that in certain embodiments, the active hormone peptide derived from the preprohormone of the invention comprises at least one of the first or the second peptide fragments according to the invention.

According to more specific embodiments the method of the invention comprises the step of administering an effective amount of at least one of: the isolated active hormone peptides of the invention (derived from the preprohormones disclosed herein). Such active hormone peptide comprise at least one of the first or the second peptide fragments according to the invention. Non limiting examples for such active hormone peptides are the peptides as denoted by any one of SEQ ID NO. 42, 40, 41, 43, 53, 54, 57, 58, 61, 62, 65, 68, 69, 72, 73, 76, 77, 93, 98, 99, 100, 101, 102, 103, 104, 105 and 106, any analogues, derivatives or variants thereof. In yet another embodiment, the method of the invention uses for administration any analogs of the active hormone peptides of the invention, specifically, as denoted by any one of SEQ ID NO. 44, 46 and 45. Alternatively, the method of the invention uses as an active ingredient any of the isolated preprohormone precursor peptides as denoted by any one of SEQ ID NO. 49, 50, 52, 56, 60, 64, 67, 71, 75, 79, 81, 83, 85, 89, 87, and 91 and any homologs, analogs and derivatives thereof. In other embodiments, the method of the invention may use as an active ingredient any nucleic acid sequence encoding said preprohormone, as denoted by any one of SEQ ID NO. 4, 5, 51, 55, 59, 63, 66, 70, 74, 78, 80, 82, 84, 86, 88 and 90, and any combinations thereof.

According to one specific embodiment, the method of the invention may comprise the step of administering as an active ingredient an effective amount of any of the active hormone peptides of the invention. As noted herein before, each of the active hormone peptides of the invention comprise at least one of the first or the second peptide fragments defined herein before.

According to another specific embodiment, the method of the invention may use at least one active hormone peptide that comprises the first peptide fragment according to the invention. More specifically, the method of the invention may comprise the step of administering an effective amount of at least one trideca NKF active hormone peptides of the invention, as denoted by any one of SEQ ID NO. 42, 43, 53, 57, 61, 65, 68, 72, 76, 98, 100, 102, 103 and 105, any combinations thereof or any analogues, variants or derivatives thereof. Certain non-limiting examples for such NKF analogs may be the analog as denoted by SEQ ID NO. 46.

In yet more specific embodiments, the method of the invention may comprise the step of administering an effective amount of an active hormone peptide of the invention designated NKFa. In further specific embodiments, this peptide is a trideca peptide that comprises the amino acid sequence of Tyr-Asn-Asp-Ile-Asp-Tyr-Asp-Ser-Phe-Val-Gly-Leu-Met-NH₂, as denoted by SEQ ID NO:42, or any analogs and derivatives thereof.

In another specific embodiment, the method of the invention may use for administration an effective amount of an analog of the NKFa trideca active hormone peptide of the invention. A specific and non limiting example for such trideca peptide analog is the Succ-Asp-Ser-Phe-N(Me)Val-Gly-Leu-Met-NH₂ analog as denoted by SEQ ID NO:46.

In yet another specific embodiment, the method of the invention may comprise the step of administering an effective amount of the active hormone peptide designated NKF b. In certain embodiments, such active hormone peptide is a trideca peptide comprising the amino acid sequence as denoted by SEQ ID NO. 43.

In yet other alternative embodiments, the invention provides methods comprising the step of administering to said fish an effective amount of at least one active hormone peptide that comprises the second peptide fragment as defined by the invention. Such active hormone peptides are designated by the invention as NKB peptides and may comprise the amino acid sequence of any one of SEQ ID NO. 40, 41, 54, 58, 62, 69, 73, 77, 93, 99, 101, 104 and 106. In yet another specific embodiment, the method of the invention may comprise the step of administering an effective amount of an NKB active hormone peptide, specifically, the NKBa that comprises the amino acid sequence of Glu-Met-His-Asp-Ile-Phe-Val-Gly-Leu-Met NH₂, as denoted by SEQ ID NO:40, and any analogues and derivatives thereof.

In another particular embodiment, the method of the invention may comprise the step of administering an analog of the NKBa peptide of SEQ ID NO. 40. A non-limiting example for such peptide analog is the Succ-Asp-Ile-Phe-N(Me)Val-Gly-Leu-Met-NH₂, as denoted by SEQ ID NO: 44.

According to another specific embodiment, the method of the invention may use an effective amount of at least one active hormone peptide designated NKBb. In one specific embodiment, this peptide comprises the amino acid sequence of Ser-Thr-Gly-Ile-Asn-Arg-Glu-Ala-His-Leu-Pro-Phe-Arg-Pro-Asn-Met-Asn-Asp-Ile-Phe-Val-Gly-Leu-Leu-NH₂, as denoted by SEQ ID NO:41, or any analogues and derivatives thereof.

In another specific embodiment, the method of the invention may comprise the step of administering an effective amount of an analogue of the active hormone peptide NKBb of the invention. A non-limiting example for such analogue is Succ-Asp-Phe-N(Me)Val-Gly-Leu-Met-NH₂ denoted by SEQ ID NO:45.

According to specific embodiments, the method of the invention is particularly applicable for regulating reproduction in fish, specifically, any stage of the reproduction process in fish. In more specific embodiments, regulation of reproduction in fish may comprise at least one of: advancing the onset of puberty, regulating the timing and amount of ovulation and spawning, synchronization or stimulation of reproduction, enhancing the development of gammets, enhancing vitellogenesis, induction of Gonadotropin-releasing hormone (GnRH), increasing the level of Luteinizing-hormone (LH), Follicle-stimulating hormone (FSH) or of any other hormone or any hypothalamic neuropeptide or neurohormone (for example, kisspeptin1, kisspeptin2, oxcytocin, Neuropeptide Y, melanocyte-stimulating hormones), induction of the Kisspeptine pathway and induction of oocyte maturation.

It should be noted that the biological effect of any of the methods of the invention are as described herein before for other aspects of the invention, namely, for the composition aspect. More specific embodiments of the invention relate to method for enhancement of different stages of reproduction of fish, as described herein before.

In the methods of the invention, administration may be by any of route known to a person skilled in the art, such as, but not limited to the following routes: oral administration, intravenous, intramuscular, intraperitoneal, intrathecal or subcutaneous injection; intrarectal administration; sustained release, soaking, feeding or drinking or topical administration or ocular administration. “Sustained release” is understood to mean a gradual release of active compound in a controlled manner. Such sustained release formulations of active compounds may be solid and may be prepared in any suitable form such as pellets, discs or rods, or encapsulated in microspheres. Active compounds may be also administered by methods including implementation of a unit of active compound in any suitable form, such as long lasting implants. Methods of administration also include capsulization and intracerebroventricular (i.c.v.).

In certain embodiments, the method of the invention comprises the step of administering an effective amount of at least one of an isolated active hormone peptides of the invention, any analogues thereof or any of the preprohormone peptides of the invention. In specific embodiments where the active ingredient is any of the active hormone peptides of the invention, specifically, any one of the NKF peptides or the NKB peptides and any analogues thereof, an effective amount of such active ingredient may range between about 0.1 to about 100 pmol/g body weight (BW), specifically, between about 20 to about 25 pmol/gBW.

In still further specific embodiments, the method of the invention may comprise the step of administering an effective amount of at least one of the active hormone peptides of the invention, specifically, any one of the NKF peptides or the NKB peptides and any analogues thereof, that may range between about 0.05 to about 500 μg/Kg body weight BW), between about 0.5 to about 5 μg/Kg BW or between about 20 to about 25 μg/Kg BW.

In yet another aspect, the present invention provides the use of an effective amount of at least one of an isolated preprohormone, that is the precursor peptide of the invention, any active hormone peptide derived therefrom, any analogues thereof, or any nucleic acid sequence encoding the preprohormone molecule of the invention, and any combinations thereof, in the preparation of a composition for regulating reproduction in fish. In more specific embodiments the preprohormone of the invention comprises a first and a second peptide fragments:

(a) the first peptide fragment comprises the amino acid sequence of:
X¹-X²-X³-X⁴-X⁵-X⁶-Asp⁷-X⁸-Phe⁹-Val¹⁰-X¹¹-Leu¹²-Met¹³, as denoted by SEQ ID NO. 96, Wherein:
X¹, is Tyr or any hydrophobic, a polar or positively charged amino acid selected from Phe, Ser, Lys and Gln;
X², is Asn or Ser or a hydrophobic, a polar, an acidic or positively charged amino acid selected from His, Thr, Asp, Arg, Lys and Ile;
X³, is Asp or a hydrophobic or a polar amino acid selected from Phe and Arg;
X⁴, is Ile or Leu or a polar or a hydrophobic amino acid selected from Asp, Tyr and Val;
X⁵, is Asp or an hydrophobic amino acid Met;
X⁶, is Tyr or acidic amino acid selected from Asp or His;
X⁸, is Ser or a polar or hydrophobic amino acid selected from Thr, Phe and Val;
X¹¹, is Gly or a polar amino acid, Ser;
X¹³, is Met or a hydrophobic amino acid Leu, and
(b) the second peptide fragment comprises the amino acid sequence of:
Glu¹-Met²-His³-Asp⁴-Ile⁵-Phe⁶-Val⁷-Gly⁸-Leu⁹-Met¹⁰ as denoted by SEQ ID NO. 40 and variants thereof. More specifically, the variants may comprise a substitution in at least one position selected from a group consisting of:
Glu¹ may be any one of Asn, Asp and Tyr; His³ may be substituted with any one of Asn and Asp; Asp⁴ may be Gln; Ile⁵ may be substituted with Val; Phe⁶ may be Leu; Val⁷ may be Ile; Gly⁸ may be substituted with Ala; and Met¹⁰ may be replaced with Leu.

It should be noted that in certain embodiments, the active hormone peptide derived from the preprohormone of the invention and used by the invention, comprises at least one of the first or the second peptide fragments according to the invention. In optional embodiments, the composition of the invention may further comprise a pharmaceutically acceptable carrier, excipient or diluent.

According to certain embodiments, the invention provides the use of at least one of: the isolated active hormone peptides derived from the preprohormones of the invention. Such active hormone peptide comprise at least one of the first or the second peptide fragments according to the invention. Non limiting examples for such active hormone peptides are the peptides as denoted by any one of SEQ ID NO. 42, 40, 41, 43, 53, 54, 57, 58, 61, 62, 65, 68, 69, 72, 73, 76, 77, 93, 98, 99, 100, 101, 102, 103, 104, 105 and 106, any analogues, derivatives or variants thereof. In yet another embodiment, the invention provides the use of an analogues of the active hormone peptides of the invention, specifically, as denoted by any one of SEQ ID NO. 44, 46 and 45. Alternatively, the use of any of the preprohormone peptides of the invention (or precursor peptide) as denoted by any one of SEQ ID NO. 49, 50 52, 56, 60, 64, 67, 71, 75, 79, 81, 83, 85, 89, 87, and 91 and any homologs, analogs and derivatives thereof. In other embodiments, the invention provides the use of any nucleic acid sequence encoding said preprohormone, as denoted by any one of SEQ ID NO. 4, 5, 51, 55, 59, 63, 66, 70, 74, 78, 80, 82, 84, 86, 88 and 90, and any combinations thereof, in the preparation of a composition for regulating any stage of reproduction in fish.

It should be appreciated that the invention further encompasses the use of any variant, homolog or derivative of any of the peptides or the nucleic acid sequences described by the invention, for the preparation of said composition.

According to one specific embodiment, the invention provides the use of an effective amount of any of the active hormone peptides of the invention. As noted herein before, each of the active hormone peptides of the invention comprise at least one of the first or the second peptide fragments defined herein before.

According to another specific embodiment, the invention provides the use of at least one active hormone peptide that comprises the first peptide fragment according to the invention. More specifically, the use according to the invention may comprise an effective amount of at least one trideca NKF active hormone peptides of the invention, as denoted by any one of SEQ ID NO. 42, 43, 53, 57, 61, 65, 68, 72, 76, 98, 100, 102, 103 and 105, any combinations thereof or any analogues, variants or derivatives thereof. Certain non-limiting examples for such analogs may be the analog as denoted by any one of SEQ ID NO. 46.

In yet more specific embodiments, the invention provides the use of an active hormone peptide of the invention designated NKFa. In further specific embodiments, this peptide is a trideca peptide that comprises the amino acid sequence of Tyr-Asn-Asp-Ile-Asp-Tyr-Asp-Ser-Phe-Val-Gly-Leu-Met-NH₂, as denoted by SEQ ID NO:42, or any analogs and derivatives thereof.

In another specific embodiment, the invention provides the use of an analog of the NKFa trideca active hormone peptide of the invention. A specific and non limiting example for such trideca peptide analog is the Succ-Asp-Ser-Phe-N(Me)Val-Gly-Leu-Met-NH₂ analog as denoted by SEQ ID NO:46.

In yet another specific embodiment, the invention provides the use of the active hormone peptide designated NKF b. In certain embodiments, such active hormone peptide is a trideca peptide comprising the amino acid sequence as denoted by SEQ ID NO. 43.

In yet another embodiment, the invention provides the use of at least one active hormone peptide that comprises the second peptide fragment as defined by the invention. Such active hormone peptides are designated by the invention as NKB peptides and may comprise the amino acid sequence of any one of SEQ ID NO. 40, 41, 54, 58, 62, 69, 73, 77, 93, 99, 101, 104 and 106. In yet another specific embodiment, the use of the invention concerns an NKB active hormone peptide, specifically, the NKBa that comprises the amino acid sequence of Glu-Met-His-Asp-Ile-Phe-Val-Gly-Leu-Met NH₂, as denoted by SEQ ID NO:40, and any analogues and derivatives thereof.

In another particular embodiment, the invention provides the use of an analog of the NKBa peptide of SEQ ID NO. 40. A non-limiting example for such peptide analog is the Succ-Asp-Ile-Phe-N(Me)Val-Gly-Leu-Met-NH₂, as denoted by SEQ ID NO: 44.

Still further, the invention provides the use of at least one active hormone peptide designated NKBb. In one specific embodiment, this peptide comprises the amino acid sequence of Ser-Thr-Gly-Ile-Asn-Arg-Glu-Ala-His-Leu-Pro-Phe-Arg-Pro-Asn-Met-Asn-Asp-Ile-Phe-Val-Gly-Leu-Leu-NH₂, as denoted by SEQ ID NO:41, or any analogues and derivatives thereof.

In another specific embodiment, the use of the invention relates to an analogue of the active hormone peptide NKBb of the invention. A non-limiting example for such analogue is Succ-Asp-Phe-N(Me)Val-Gly-Leu-Met-NH₂ denoted by SEQ ID NO:45.

According to specific embodiments, the invention provides the use of any of the peptides described herein above for the preparation of a composition for regulating reproduction in fish, specifically, any stage of the reproduction process in fish. In more specific embodiments, regulation of reproduction in fish may comprise at least one of: advancing the onset of puberty, regulating the timing and amount of ovulation and spawning, synchronization or stimulation of reproduction, enhancing the development of gammets, enhancing vitellogenesis, induction of Gonadotropin-releasing hormone (GnRH), increasing the level of Luteinizing-hormone (LH), Follicle-stimulating hormone (FSH) or of any other hypothalamic neuropeptide or neurohormone, induction of the Kisspeptine pathway and induction of oocyte maturation.

In yet another aspect, the present invention provides at least one of: an isolated preprohormone, any active hormone peptide derived therefrom, any analogues thereof, or any nucleic acid sequence encoding the preprohormone molecule of the invention, and any combinations thereof for use in a method for regulating reproduction in fish.

In more specific embodiments the preprohormone of the invention comprises a first and a second peptide fragments:

(a) the first peptide fragment comprises the amino acid sequence of:
X¹-X²-X³-X⁴-X⁵-X⁶-Asp⁷-X⁸-Phe⁹-Val¹⁰-X¹¹-Leu¹²-Met¹³, as denoted by SEQ ID NO. 96, wherein:
X¹, is Tyr or any hydrophobic, a polar or positively charged amino acid selected from Phe, Ser, Lys and Gln;
X², is Asn or Ser or a hydrophobic, a polar, an acidic or positively charged amino acid selected from His, Thr, Asp, Arg, Lys and Ile;
X³, is Asp or a hydrophobic or a polar amino acid selected from Phe and Arg;
X⁴, is Ile or Leu or a polar or a hydrophobic amino acid selected from Asp, Tyr and Val;
X⁵, is Asp or an hydrophobic amino acid Met;
X⁶, is Tyr or acidic amino acid selected from Asp or His;
X⁸, is Ser or a polar or hydrophobic amino acid selected from Thr, Phe and Val;
X¹¹, is Gly or a polar amino acid, Ser;
X¹³, is Met or a hydrophobic amino acid Leu, and
(b) the second peptide fragment comprises the amino acid sequence of:
Glu¹-Met²-His³-Asp⁴-Ile⁵-Phe⁶-Val⁷-Gly⁸-Leu⁹-Met¹⁰ as denoted by SEQ ID NO. 40 and variants thereof. More specifically, the variants may comprise a substitution in at least one position selected from a group consisting of:
Glu¹ may be any one of Asn, Asp and Tyr; His³ may be substituted with any one of Asn and Asp; Asp⁴ may be Gln; Ile⁵ may be substituted with Val; Phe⁶ may be Leu; Val⁷ may be Ile; Gly⁸ may be substituted with Ala; and Met¹⁰ may be replaced with Leu.

It should be noted that other embodiments of this aspect relate to any of the peptides of the invention as described herein before.

As shown by the following Examples, the inventors isolated and identified the NKB receptor in different species of fish. Moreover, the inventors demonstrated that the active hormone peptides of the invention as well as analogues thereof, act as agonists inducing signaling (PKA and PKC signaling) through the NKB receptors. Thus, in yet another aspect, the invention provides an NKB/NKBR system for regulating reproduction in fish, wherein said system comprises at least one of:

A. at least one of an isolated preprohormone, which is the precursor peptide of the invention, any active hormone peptide derived therefrom, any analogues thereof, or any nucleic acid sequence encoding the preprohormone molecule of the invention, and any combinations thereof. In more specific embodiments the preprohormone of the invention comprises a first and a second peptide fragments:
(a) the first peptide fragment comprises the amino acid sequence of:
X¹-X²-X³-X⁴-X⁵-X⁶-Asp⁷-X⁸-Phe⁹-Val¹⁰-X¹¹-Leu¹²-Met¹³, as denoted by SEQ ID NO. 96, Wherein:
X¹, is Tyr or any hydrophobic, a polar or positively charged amino acid selected from Phe, Ser, Lys and Gln;
X², is Asn or Ser or a hydrophobic, a polar, an acidic or positively charged amino acid selected from His, Thr, Asp, Arg, Lys and Ile;
X³, is Asp or a hydrophobic or a polar amino acid selected from Phe and Arg;
X⁴, is Ile or Leu or a polar or a hydrophobic amino acid selected from Asp, Tyr and Val;
X⁵, is Asp or an hydrophobic amino acid Met;
X⁶, is Tyr or acidic amino acid selected from Asp or His;
X⁸, is Ser or a polar or hydrophobic amino acid selected from Thr, Phe and Val;
X¹¹, is Gly or a polar amino acid, Ser;
X¹³, is Met or a hydrophobic amino acid Leu, and
(b) the second peptide fragment comprises the amino acid sequence of:
Glu¹-Met²-His³-Asp⁴-Ile⁵-Phe⁶-Val⁷-Gly⁸-Leu⁹-Met¹⁰ as denoted by SEQ ID NO. 40 and variants thereof. More specifically, the variants may comprise a substitution in at least one position selected from a group consisting of:
Glu¹ may be any one of Asn, Asp and Tyr; His³ may be substituted with any one of Asn and Asp; Asp⁴ may be Gln; Ile⁵ may be substituted with Val; Phe⁶ may be Leu; Val⁷ may be Ile; Gly⁸ may be substituted with Ala; and Met¹⁰ may be replaced with Leu; and
B. at least one NKB receptor or any nucleic acid sequence encoding the same, wherein said receptor comprises an amino acid sequence of any one of SEQ ID NO. 94 and 95 and any homologues and derivatives thereof.

Still further, the invention provides in another of its aspects, a piscine NKB receptor comprising the amino acid sequence of any one of SEQ ID NO. 94 and 95 or any analogs, derivatives, fragments or homologs thereof.

It should be noted that NKB is currently designated Tachykinin 3 gene (TAC3) in humans, Tac3 in nonhuman primates, cattle and dogs and Tac2 in rodents. As the genes encoding neurokinin B (NKB) vary among different species (e.g. TAC3 or Tac2), the mRNA products of the gene encoding NKB are herein referred to as “tac3 mRNA” and the resulting peptides are herein referred to as “NKB”. The receptor that binds NKB, termed “NK3R” in humans, is herein referred to as “tac3r” at the mRNA level and “Tac3r” at the protein level.

Disclosed and described, it is to be understood that this invention is not limited to the particular examples, methods steps, and compositions disclosed herein as such methods steps and compositions may vary somewhat. It is also to be understood that the terminology used herein is used for the purpose of describing particular embodiments only and not intended to be limiting since the scope of the present invention will be limited only by the appended claims and equivalents thereof.

It must be noted that, as used in this specification and the appended claims, the singular forms “a”, “an” and “the” include plural referents unless the content clearly dictates otherwise.

Throughout this specification and the Examples and claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The following examples are representative of techniques employed by the inventors in carrying out aspects of the present invention. It should be appreciated that while these techniques are exemplary of preferred embodiments for the practice of the invention, those of skill in the art, in light of the present disclosure, will recognize that numerous modifications can be made without departing from the spirit and intended scope of the invention.

### EXAMPLES

Without further elaboration, it is believed that one skilled in the art can, using the preceding description, utilize the present invention to its fullest extent. The following preferred specific embodiments are, therefore, to be construed as merely illustrative, and not limitative of the claimed invention in any way.

Standard molecular biology protocols known in the art not specifically described herein are generally followed essentially as in Sambrook & Russell, 2001.

Standard medicinal chemistry methods known in the art not specifically described herein are generally followed essentially in the series “Comprehensive Medicinal Chemistry” by various authors and editors, published by Pergamon Press.

### Experimental Procedures

### Animals

Wild-type zebrafish (D. rerio) were purchased from a commercial supplier (A & H Holdings, Israel) and were maintained in a dedicated zebrafish facility. Tilapia (Oreochromis niloticus) were bred at the fish facility of the Hebrew University. Mature female carp were bred at local Kibbutzim. Adult wild type fish were maintained at 27-28′C on a 14 h:10 h (light:dark) cycle, and fed twice daily, by a range of dry fish food and/or artemia. Embryos were generated from natural crosses by breeding male/female pairs. Fertilized eggs were raised in embryo medium at 28′C. All experimental procedures were approved by the Hebrew University Administrative Panel for Laboratory Animal Care.

### Data Mining, Phylogenetic Analysis and Chromosomal Synteny

Putative Tac3 gene sequences were isolated from zebrafish using a stepwise evolutionary strategy. First, by running a protein blast, using the mouse Tac2 protein (NP_{—}033338.2 denoted by SEQ ID NO:1) as input. The lowest scoring sequence that still had the neurokinin signature of FxGLM (XP_{—}001365310, denoted by SEQ ID NO:2, monodelphis domesitca) was used as input for a genomic search against the platypus genome. One region was found (termed ornAna1, Contig39139:6403-6445), which translated to the amino acid sequence GDMHDFFVGLMGKR (denoted by SEQ ID NO:3). This sequence was used as input to translated blast of fish DNA and EST sequences. Several ESTs were found that were built into two consensus contigs [the zebrafish Tac3a, denoted by SEQ ID NO:4 (NCBI accession no. JN392856) and the zebrafish Tac3b, denoted by SEQ ID NO:5 (NCBI accession no. JN392857)], aligning to two distinct regions in the zebrafish genome (chr 23 and 6, respectively). The cDNAs were cloned based on the EST contigs, and the sequence has been submitted to the Genbank. Due to the fact that these genes had more than one putative active peptide, and to the difference in sequence between the mammalian and fish sequences, many additional fish and mammalian sequences were isolated, in order to verify the identification of homology (as opposed to paralogy), thereby additional fish Tac genes were found.

Zebrafish tac3ra (tac3 receptor a, NCBI accession no. JF317292, denoted by SEQ ID NO:6) and tac3rb (tac3 receptor b, NCBI accession no. JF317293, denoted by SEQ ID NO:7) sequences were cloned based on the predicted gene sequences in the Genbank. The Tac3rc was found in a genomic search, and once again, more sequences were then sought, in order to ensure proper classification of the receptors. Thereby, additional 13 fish receptors were found. Phylogenetic analysis was then performed using both Neighbor-Joining (ClustalW) and Phylip (ProML) on the basis of alignments performed both by ClustalW and Muscle. The results were the same in all combinations of multiple alignment and tree construction programs. Bootstrapping of 1000 was performed on the NJ and of 100 on the ML trees. Synteny was observed using the UCSC genome browser and the following genome builds: human: hg19, zebrafish: Zv9/danRer7, Medaka: oryLat2, Tetraodon: tetNig2, and Fugu: fr2.

### Isolation of Zebrafish Tac3 Ligands and Receptors

Based on the in silico study of putative zebrafish Tac3 ligands (as described above) and on the predicted sequences of zebrafish Tac3 receptors, specific primers were designed for the cloning of Tac3 and Tac3 receptors (Table 1, below). The fragments were PCR-amplified from adult brain zebrafish cDNA library using Advantage 2 PCR System (Clontech, CA), used according to the manufacturer's recommendations. The PCR products were then cloned into pGEM-T-easy vector (Promega Corp, Madison, Wis.). The nucleotide sequences of the cloned fragments were obtained with T7 and SP6 primers at the Weizmann Institute Sequencing Unit (Rehovot, Israel).

TABLE 1
Primers used for cloning, quantitative real-time PCR and in situ hybridization

| SEQ ID NO. | Primer | Position | 5′ to 3′ sequence | lope | 2 | Application |
|---|---|---|---|---|---|---|
| 8 | zf ef1a- | 1,237 | aagacaaccccaaggctctca | 3.708 | .999 | Quantitative |
| | 1237F | | | | | |
| 9 | zf ef1a- | 1,491 | cctttggaacggtgtgattga | | | real-time |
| | 1419R | | | | | PCR |
| 10 | GnRH2- | 36 | gctgatgctgtgtctgagt | 3.337 | .994 | |
| | 36F | | | | | |
| 11 | GnRH2- | 196 | tgtcttgaggatgtttcttc | | | |
| | 196R | | | | | |
| 12 | GnRH3- | 47 | gtgtgttggaggtcagtct | 3.103 | .997 | |
| | 47F | | | | | |
| 13 | GnRH3- | 208 | tccacctcattcactatgtg | | | |
| | 208R | | | | | |
| 14 | kiss1-10F | 158 | acagacactcgtcccacagatg | 3.468 | .991 | |
| 15 | kiss1- | 357 | caatcgtgtgagcatgtcctg | | | |
| | 210R | | | | | |
| 16 | kiss2- | 137 | gcgttttctgtcaatggag | 3.475 | .998 | |
| | 137F | | | | | |
| 17 | kiss2- | 317 | cgcttcgtttctctttccg | | | |
| | 317F | | | | | |
| 18 | kiss1ra- | 856 | cctaacttcaaggccaac | 3.424 | .987 | |
| | 856F | | | | | |
| 19 | kiss1ra- | 1,095 | cctctcagtgttgctttc | | | |
| | 1095R | | | | | |
| 20 | kiss1rb- | 755 | agacgtcatcggagcgtg | 3.305 | .954 | |
| | 755F | | | | | |
| 21 | kiss1rb- | 1,041 | cctccttttgaagatcagaggac | | | |
| | 1041R | | | | | |
| 22 | zf tac3a- | 29 | tggttttggtgctggaaacc | 3.513 | .997 | |
| | F29 | | | | | |
| 23 | zf tac3a- | 191 | tctgtttcggcgtttctgc | | | |
| | R191 | | | | | |
| 24 | zf tac3b- | 86 | ctccttcactg acaacagcgac | 3.239 | .988 | |
| | F86 | | | | | |
| 25 | zf tac3b- | 246 | gtttctccgtcctaacagtccg | | | |
| | R246 | | | | | |
| 26 | zf tac3ra | 154 | gctcataagcggatgcgaac | 3.502 | .969 | |
| | F154 | | | | | |
| 27 | zf tac3ra | 334 | tggcaaacacggaggtgac | | | |
| | R334 | | | | | |
| 28 | zf tac3rb | 343 | tccatgacagcgattgcagt | 3.322 | .964 | |
| | F343 | | | | | |
| 29 | zf tac3rb | 523 | cgtagcaaatggttctgcg ag | | | |
| | R523 | | | | | |
| 30 | zf tac3a R- | −122 | ccctgtctctgtgtcttgtctg | | | In situ |
| | 122 | | | | | |
| 31 | zf tac3a | 360 | gcctataacccacgacgaaac | | | hybridization/ |
| | R360 | | | | | |
| 32 | zf tac3b F- | −17 | ggataaggtgtgcgaggatg | | | Cloning |
| | 17 | | | | | |
| 33 | zf tac3b | 382 | tcatacaccacagcaaaacctcag | | | |
| | stop | | | | | |
| 34 | zf tac3Ra | 1 | atggcacagtcacagaacgg | | | Cloning |
| | start | | | | | |
| 35 | zf tac3Ra | 1,180 | tcaggagaattcctccttcg | | | |
| | stop | | | | | |
| 36 | zf tac3Rb | 1 | atggctggtcctcagagcgg | | | |
| | start | | | | | |
| 37 | zf tac3Rb | 1,161 | tcagctgagctgctctgttgc | | | |
| | stop | | | | | |

### Tissue Distribution and Expression Toward Puberty

Analysis of tissue distribution of zebrafish tac3a, tac3b, tac3ra and tac3rb (denoted by SEQ ID NO:4, 5, 6 and 7, respectively) was carried out by real-time PCR, as detailed below and as described by Biran et al. [4]. Tissue samples were collected from sexually mature postvitellogenic female and milt-producing male zebrafish (males: body weight (BW) 533.3±170.1 mg; body length (BL) 40.67±4.16 mm; gonado-somatic index (GSI) 15.3%±3.2%; females: BW, 836.67±145.72 mg; BL, 39.33±1.53 mm; GSI, 16.23%±2.52%). Total RNA (1 μg) was extracted from each of the following tissues: brain, pituitary, spleen, gill, kidney, intestine, pancreas, muscle, adipose tissue, liver, ovary and testis and cDNA samples were prepared according to Levavi-Sivan et al. 2004 [9]. The tissue expression patterns of Zebrafish tac3a, tac3b, tac3ra, and tac3rb mRNA in the various zebrafish tissues were analyzed by real-time PCR with specific primer sets as listed in Table 1, above (denoted by SEQ ID NO:8 SEQ ID NO:37).

To study the gene expression of the NKB/NKBR system at different fish ages, 15 fish were sampled at each of the ages 2, 4, 6, 8, and 12 weeks post fertilization. The fish were taken randomly from at least four independent tanks. The brain was removed, the remaining body was fixed in Bouin's fluid (Sigma), and the pubertal stage classification was determined by histology of the gonads under light microscopy, as described by Biran et al. 2008 [4].

### Real-Time PCR

Real-time PCR of zebrafish tac3a, tac3b, tac3ra and tac3rb (denoted by SEQ ID NO:4, 5, 6 and 7, respectively) was generally performed according to Levavi-Sivan et al., 2006 [10] and Biran et al., 2008 [4]. Since Elongation factor 1α (ef1α) was recently shown to be a suitable reference gene, both for tissue analysis and for developmental time studies of zebrafish [11], it was used in the present study as a reference gene. The primers, R² value and slope, calculated by linear regression for all of the genes tested, are described in Table 1, above.

### Whole Mount In Situ Hybridization Analysis of Embryos and Adults

A fragment at the size of 502 bp (position −122 to 380 in Genbank accession no. JN392856, denoted by SEQ ID NO. 38) of zebrafish tac3a was cloned into pGEM-T easy vector. Then, antisense and sense riboprobes were synthesized (Digoxigenin (Dig) RNA labeling kit, Roche Diagnostics, Basel, Switzerland), using SpeI or NcoI linearized plasmids, respectively, as a template. Similarly, riboprobes were synthesized for tac3b, tac3ra and tac3rb. Whole mount in situ hybridization was conducted as previously described in Palevitch et al., 2007 [12]. Briefly, fixed embryos and larvae at various stages of development (1-12 Days post fertilization (dpf)) were treated with ice cold acetone and proteinase K (10 μg/ml in PBS) and then hybridized overnight at a temperature of 65° C. with hybridization buffer containing 1 ng/μl tac3a probe. Following washing, samples were incubated for 3 hours with anti-DIG antibody conjugated to alkaline phosphatase (AP; 1:5000; Roche). The riboprobe-antibody complex was detected by the enzymatic reaction of AP with a chromogenic substrate (BM Purple AP substrate, Roche). The heads of larvae were photographed from the dorsal and lateral projection using an Olympus dissecting microscope (SZX12, Olympus, Japan) equipped with digital camera (DP70, Olympus).

### In Situ Hybridization Analysis in Adult Zebrafish

In situ hybridization was conducted as described in Mitani et al., 2010 [13] with slight modifications. Briefly, fish (0.6-0.8 g sexually mature zebrafish) were first anesthetized with MS-222 (Sigma, St. Louis, Mo.) and decapitated. Brains were removed and fixed with 4% paraformaldehyde in PBS for 6 h at 4° C. and immersed in PBS containing 20% sucrose and 30% O.C.T [Optimal cutting tissue] (Sakura, Tokyo, Japan), for 24 h. Brains were then embedded in O.C.T, frozen in liquid nitrogen, sectioned frontally at 12 μm on a cryostat at −18° C., and mounted onto Superfrost plus glass slides (Thermo scientific, Waltham, Mass.). In order to detect tac3a and tac3b mRNA, a specific digoxigenin (DIG)-labeled riboprobe for tac3a (position 122-360 in GenBank accession no. JN392856, were prepared, denoted by SEQ ID NO: 3) Probes were prepared using DIG RNA labeling kit (SP6/T7; Roche, Molecular Biochemicals GmbH, Mannheim, Germany).

Brain sections were washed twice in PBS, treated with 1 μg/ml protease K for 15 min at 37° C., post-fixed with 4% paraformaldehyde in PBS for 15 min, and incubated with 0.25% acetic anhydride in 0.1M triethanolamine, for 10 min. Then the sections were prehybridized at 58° C., for 1 h in hybridization buffer, containing 50% formamide, 5× saline sodium citrate (SSC), 0.12 M phosphate buffer (pH 7.4) and 100 μg/ml tRNA. Slides were incubated at 58° C. overnight, in the same solution, containing 1 μg/ml denatured riboprobe. Diethyl pyrocarbonate-treated water was used for the preparation of all solutions for treatment before hybridization.

After hybridization, sections were washed twice with 50% formamide and 2×SSC followed by two washes of 2×SSC and two washes of 0.5×SSC for 15 min each at 58° C. Slides were immersed in DIG-1 (0.1 M Tris-HCl, 0.16 M NaCl, and 0.1% Tween 20) for 5 min, 1.5% blocking reagent with DIG-1 for 30 min, and DIG-1 for 15 min, and then incubated with an alkaline phosphatase-conjugated anti-DIG antibody (diluted 1:1000 with DIG-1; Roche) for at least 2 h. Sections were washed with DIG-1 twice for 15 min each, and DIG-3 (0.1 M Tris-HCl, pH 9.5; 0.1 M NaCl; 0.05 M MgCl₂) for 5 min. Sections were then treated with a chromogenic substrate NBT/BCIP stock solution (Roche, Mannheim, Germany) diluted 1:250 in DIG-3 until a visible signal was detected. Sections were immersed in a reaction stop solution (10 mM Tris-HCl, pH8.0; 1 mM EDTA, pH8.0) to stop the chromogenic reaction. Sections were then dehydrated, covered using ClearMount™ Mounting Solution (Invitrogen, Paisley, UK) and examined using light microscopy.

### Peptide Synthesis

Zebrafish Tac3a, also termed herein as NKBa (EMHDIFVGLM-NH₂, denoted by SEQ ID NO:40), Zebrafish Tac3b, also termed herein as NKBb (STGINREAHLPFRPNMNDIFVGLLEMHDIFVGLM-NH₂, denoted by SEQ ID NO:41) and Zebrafish Tac3f, also termed herein as NKF (YNDIDYDSFVGLM-NH₂, denoted by SEQ ID NO:42) were synthesized with the automated solid-phase method by applying Fmoc active ester chemistry. It was subsequently purified by HPLC to a purity level >95% (GeneMed, USA). The carboxy terminus of each peptide was amidated.

Synthesis of peptide analogues, namely NKBa-analogue, NKBb-analogue and NKF-analogue was performed as follows. The design of the novel fish NKB agonists was based on the structure of the highly selective known NKB agonist, Senktide (Succ-Asp-Phe-N(Me)Phe-Gly-Leu-Met-NH2, also denoted by the SEQ ID NO:47), which is the most active and selective NK3R agonist discovered so far (EC₅₀ GPI, nM: NK-1=35,000, NK-2>200,000, NK-3=0.5). The novel fish NKB agonists were prepared by extensive structure-activity relationship (SAR) studies of the mammalian NKB (i.e. DMHDFFVGLM-NH2, denoted by SEQ ID NO:48). The design of the fish NKB agonists was based on similar considerations as those that were taken in the development of Senktide, namely omitting the N-terminal sequence up to Asp (D) and replacing the amino acid presiding Gly (G) with its N-Me analog. It was also shown that replacement of Phe8 by MePhe8 and replacement of the amino terminal tripeptide by succinyl residue imposed selectivity to the NKBR receptor (NK3R).

By applying the above principles, the following fish NKB peptides, termed NKBa-analog, NKBb-analog and NKF-analog, as shown in Table 2 below, were synthesized:

TABLE 2
NKB analogues

| | | SEQ |
|---|---|---|
| | | ID |
| Peptide | peptide sequence | NO. |
| NKBa- | Succ-Asp-Ile-Phe-N(Me)Val-Gly-Leu- | 44 |
| analog | Met-NH2 | |
| NKBb- | Succ-Asp-Phe-N(Me)Val-Gly-Leu-Met-NH2 | 45 |
| analog | | |
| NKF- | Succ-Asp-Ser-Phe-N(Me)Val-Gly-Leu- | 46 |
| analog | Met-NH2 | |

### Receptor Transactivation Assay and Protein Structure Modeling

In order to study the signaling pathways of the novel zebrafish NKBs, the entire coding regions of zftac3ra (denoted by SEQ ID NO:6) or zftac3rb (denoted by SEQ ID NO:7), or the cDNA clone for human NK3R (obtained from the Missouri S&T cDNA Resource Center, were inserted into pcDNA3.1 (Invitrogen). The luciferase assay was conducted according to Biran et al., 2008 [4] COS-7 cells were grown in DMEM supplemented with 10% FBS, 1% glutamine, 100 U/ml penicillin, and 100 mg/ml streptomycin (Biological Industries) under 5% CO2 until confluent. Cotransfection of either pc-zftac3ra, pc-zftac3rb or pc-Htac3r (at 3 μg/plate), a reporter plasmid (at 2 μg/plate), and pCMV-β-galactosidase (at 1 μg/plate) was carried out with FuGENE 6.0 reagent (Roche). The cells were serum starved for 36 h, stimulated with vehicle or various concentrations of either human (Sigma) or zebrafish NKBs for 6 h, and then harvested and analyzed. Lysates prepared from the harvested cells were assayed for both luciferase activity and β-galactosidase activity, which was used as an internal standard to normalize the luciferase activity directed by the test plasmid. Transfection experiments were performed in triplicate with three independently isolated sets. Protein structure prediction of zebrafish NKBa, NKBb or NKF were performed on I-Tasser servers [19, 20].

### In Vivo Effect of Exposure to Estradiol

Two-month old zebrafish were exposed (12 fish per aquarium of 3 L), by immersion, to estradiol E₂ (Sigma) at a concentration of 5 μg/liter (18 nM), or vehicle (ethanol), for 3 days. A relatively low concentration was used, since the natural concentration in the plasma of adult vitellogenic female zebrafish was determined to be 3-4 ng/ml E_{2 [}15]. The water was maintained at 28.5° C. and replaced daily. The sex of each sampled fish was confirmed by dissection. Samples were frozen instantly in liquid nitrogen and stored at −80° C. RNA extraction, reverse transcription of RNA and Real-Time PCR were carried out as described by Biran et al., (2008) and Levavi-Sivan et al., (2006) [6, 10].

### Effect of NKB Analogs on Puberty

In vivo studies involved administering NKBa, NKBb or NKF and the analogues thereof, denoted by SEQ ID NO. 40-42 and 44-46, respectively to immature fish (20 fish/treatment). The circulating levels of E2, 11KT, LH and FSH were determined prior to hormonal manipulations, in order to confirm that these fish were indeed sexually immature. The fish were injected intramuscularly (i.m.) or intraperitonealy (i.p.) at various doses. Adult female zebra fish were injected intraperitoneally with 20 pmol/g body weight. Control fish were injected with saline. Plasma samples were collected at time 0, and specific times after the administration of the peptide/s, to determine the levels of E2, 11KT, LH and FSH.

### Example 1

### Cloning Two Types of tac3 and Phylogenetic Analysis

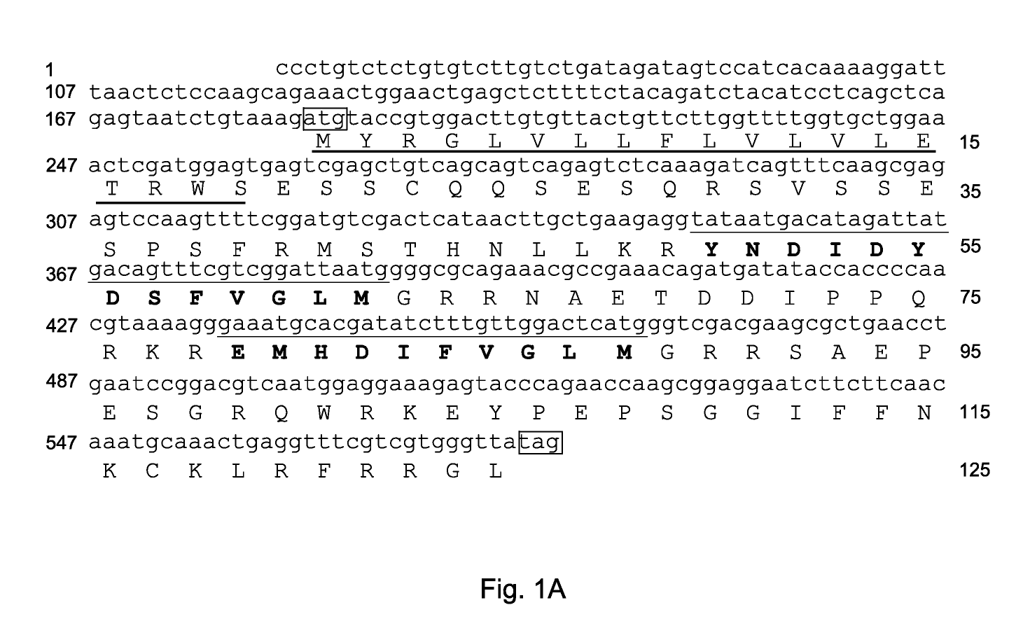

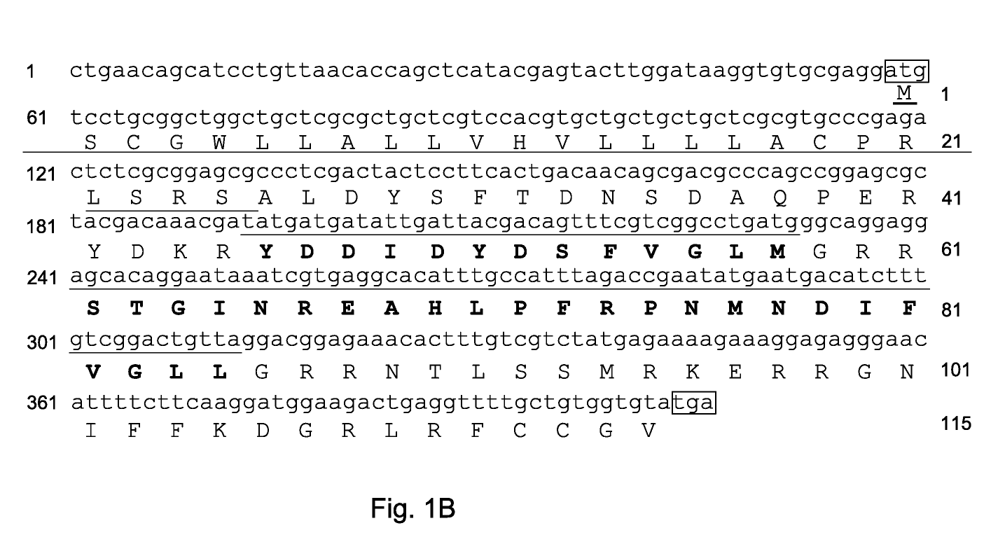

The involvement of the NKB/NKB receptors in the control of reproduction in fish was examined by taking the following steps. First, the full length tac3a (SEQ ID NO:4) and tac3b (SEQ ID NO:5) cDNA from zebrafish brain were cloned. Tac3a encodes the decapeptide (10 amino acid) sequence EMHDIFVGLM, denoted herein by SEQ ID NO:40, (see FIG. 1A, Accession No. JN392856), while tac3b encodes the 24 amino acid (aa) peptide STGINREAHLPFRPNMNDIFVGLL, herein denoted by SEQ ID NO:41 (see FIG. 1B, Accession No. JN392857), both having the tachykinin signature motif, namely, FXGLM-NH₂ as denoted by SEQ ID NO. 92, flanked by potential dibasic cleavage sites and an adjacent glycine at the C-terminus for amidation [16]. Prediction of peptides cleavage sites was conducted using NeuroPred application [3].

Typically, following the action of the prohormone convertase, a carboxypeptidase removes the C-terminal dibasic residues, and a peptidylglycine a-amidating enzyme converts the exposed glycine into a C-terminal amide [17]. Table 3 below shows that zebrafish Tac3a peptide displayed about 25% identity, at the protein level, with human or mouse TAC3, 55% with Tac3a of the salmon, and 52% with Tac3 of the Medaka. Table 3 also shows that zebrafish tac3b has only about 18% identity with the human TAC3 and mouse Tac2, 40% identity with salmon Tac3b, and 36% with that of the Medaka. The zebrafish tac3 share only 36% identity with each other.

TABLE 3
Percent amino acid sequence identities (upper right to the table diagonal) and similarities (lower left
to the table diagonal) among Tac3 of different species as determined by EMBOSS* Stretcher alignment tool

| | | | | | | | | European | | Rainbow | Arctic | | |
|---|---|---|---|---|---|---|---|---|---|---|---|---|---|
| | Zebrafish | Zebrafish | Human | Sheep | Mouse | Salmon | Salmon | Seabass | Medaka | Smelt | Cod | Frog | Aligator |
| | Tac3a | Tac3b | Tac3 | Tac3 | Tac2 | Tac3a | Tac3b | Tac3 | Tac3a | Tac3 | Tac3 | Tac3 | Tac3 |
| Zebrafish Tac3A | — | 35.7 | 25 | 18.1 | 24.8 | 55.3 | 43.9 | 27.8 | 52 | 59.2 | 50 | 31.8 | 25.4 |
| (cypriniformes) | | | | | | | | | | | | | |
| Zebrafish Tac3B | 45.2 | — | 18.2 | 19.7 | 18.9 | 40.2 | 40.4 | 30.4 | 36.3 | 40.6 | 38.3 | 30.7 | 20.5 |
| (cypriniformes) | | | | | | | | | | | | | |
| Human Tac3 | 41.7 | 34.7 | — | 55.6 | 61.1 | 24.2 | 24.4 | 13.2 | 25.2 | 25.8 | 28.6 | 29.1 | 39.2 |
| Sheep Tac3 | 37 | 38.5 | 66.7 | — | 66.7 | 23 | 18.9 | 14.2 | 24.6 | 23.4 | 26 | 34.9 | 38.5 |
| Mouse Tac3 | 45 | 37.7 | 73 | 73.3 | — | 26.9 | 27.6 | 17.2 | 24.6 | 26 | 28.5 | 36.7 | 43.9 |
| Salmon Tac3A | 71.2 | 50.8 | 43.2 | 37.8 | 41 | — | 38.2 | 23.6 | 56.1 | 65.9 | 57.2 | 40.2 | 28.6 |
| (salmoniformes) | | | | | | | | | | | | | |
| Salmon Tac3B | 62.1 | 52.9 | 34.4 | 32.6 | 38.1 | 55.9 | — | 35.1 | 38.9 | 39.9 | 34.1 | 32.1 | 20.3 |
| (salmoniformes) | | | | | | | | | | | | | |
| European Seabass | 42.1 | 43.2 | 32.6 | 30 | 33.6 | 41.4 | 43.3 | — | 24.4 | 25.6 | 26.7 | 21.8 | 18 |
| Tac3 | | | | | | | | | | | | | |
| (perciformes) | | | | | | | | | | | | | |
| Medaka Tac3A | 72.8 | 48.4 | 40.2 | 41.5 | 40.5 | 72 | 53.4 | 43 | — | 64.5 | 56.2 | 31.2 | 27.2 |
| (beloniformes) | | | | | | | | | | | | | |
| Rainbow Smelt | 74.4 | 48.4 | 43 | 39.1 | 42.5 | 77.3 | 54.2 | 41.4 | 78.2 | — | 63.4 | 36.4 | 29.6 |
| Tac3A | | | | | | | | | | | | | |
| (osmeriformes) | | | | | | | | | | | | | |
| Arctic Cod Tac3 | 68.9 | 49.2 | 42.1 | 38.9 | 43.8 | 69.6 | 54.1 | 40 | 70.8 | 73.3 | — | 34.6 | 23.4 |
| (gadiforms) | | | | | | | | | | | | | |
| Frog Tac3 | 53.5 | 40.9 | 48 | 47.3 | 50.8 | 60.6 | 49.3 | 39.1 | 55.5 | 60.5 | 54.9 | — | 36 |
| Aligator Tac3 | 45.2 | 37.7 | 56 | 54.1 | 59.3 | 45.9 | 38.3 | 33.1 | 43.2 | 47.2 | 35.9 | 51.2 | — |

— Same species, not applicable.

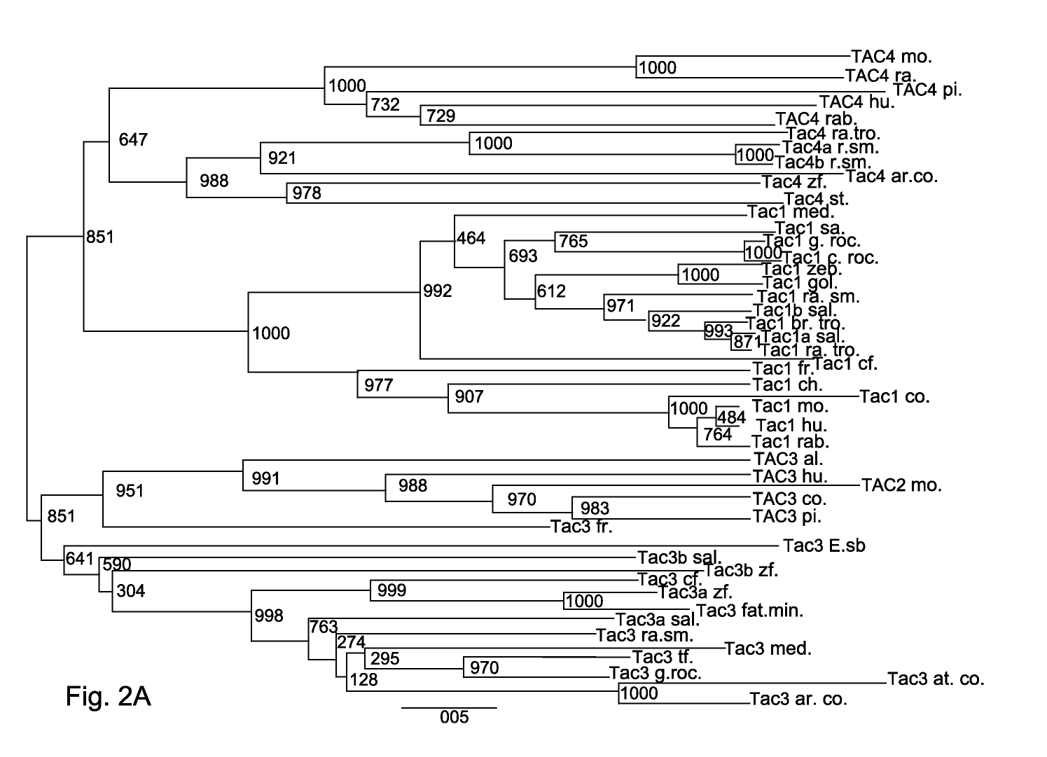

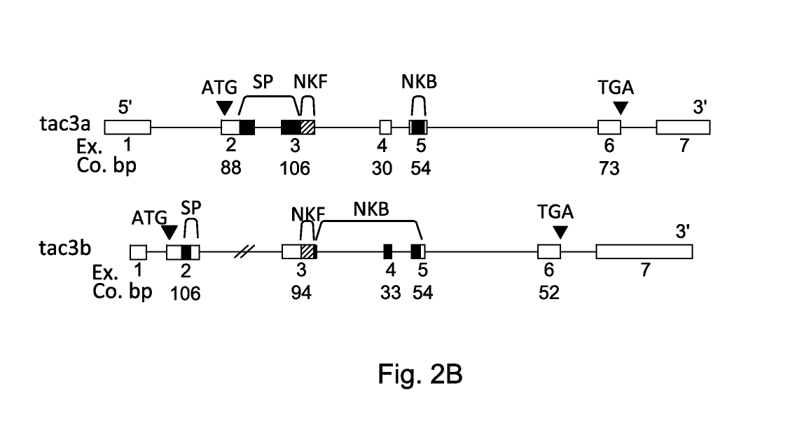

A comprehensive search for the purpose of the identification of Tac3 sequences containing the NKB peptide sequence from fish genome and ESTs known to date revealed several novel piscine (fish-related) NKBs A phylogenetic tree of all the vertebrate neurokinin genes was generated, as presented in FIG. 2A. The resulting tree showed that the vertebrate neurokinin genes identified to date fall into several distinct lineage groups. The identified Tac3 peptides from fish were grouped together with all other previously cloned or predicted Tac3 sequences from mammals, frog and alligator. The other lineage includes Tac1 from both mammals and fish that were cloned in the current study, while the third lineage included mammalian tac4 and a unique piscine group, now named Tac4 (see FIG. 2A). No precursors were found in invertebrate species that contained the exact NKB sequence [18]. The unrooted phylogenetic tree of neurokinin sequences was generated with a MEGA version 4 [32].

The in silico analyses of fish genomic structure verified that the zftac3 consists of seven exons, as shown in FIG. 2B). In mammals the tac3 gene contains seven exons, five of which are translated to form the prepro-NKB protein. Notably, zftac3a sequence was encoded in the fifth exon, while zftac3b spans exons 3 to 5 (FIG. 2B). Surprisingly, unlike in mammalian NKBs, the deduced amino acid sequence of both zebrafish tac3 genes encoded an additional putative tachykinin sequence, flanked by a Gly C-terminal amidation signal at their C termini and typical endoproteolytic sites at both termini, suggesting that novel tachykinin peptides, namely, YNDIDYDSFVGLM-NH₂ (denoted by SEQ ID NO:42) and YDDIDYDSFVGLM-NH₂ (denoted by SEQ ID NO:43), are spliced from Tac3a and Tac3b, respectively (FIGS. 1A and 1B, respectively) are produced from the precursors. Intriguingly, this additional peptide that is produced from Tac3a and Tac3b precursors was found in zebrafish, and also in all other fish species cloned in this study (11 species), however, this specific peptide could not be detected in chicken, anolis, alligator or Xenopus. These peptides possess an N-terminal dibasic cleavage site with the potential to release the peptide and the common NKB motif FVGLM at their C terminal. Hence, the novel peptides, denoted by SEQ ID NOs:42 and 43 (were generally termed neurokinin F (NKF), since they were spliced only in fish species (see FIGS. 1A, B). The NKF peptide denoted by SEQ ID NO:42 was used in the following Examples. Without wishing to be bound by theory, although this tachykinin (NKF) still exists in fish, it was probably lost during evolution in other species. Interestingly, in Tac4 there is a similar loss of one active peptide in mammals (the C-terminal peptide in Tac4 as opposed to the N-terminal peptide in Tac3), whereas most fish species retain putative active peptides in both locations.

### Example 2

### Cloning of NKB Receptors and their Phylogenetic Analysis

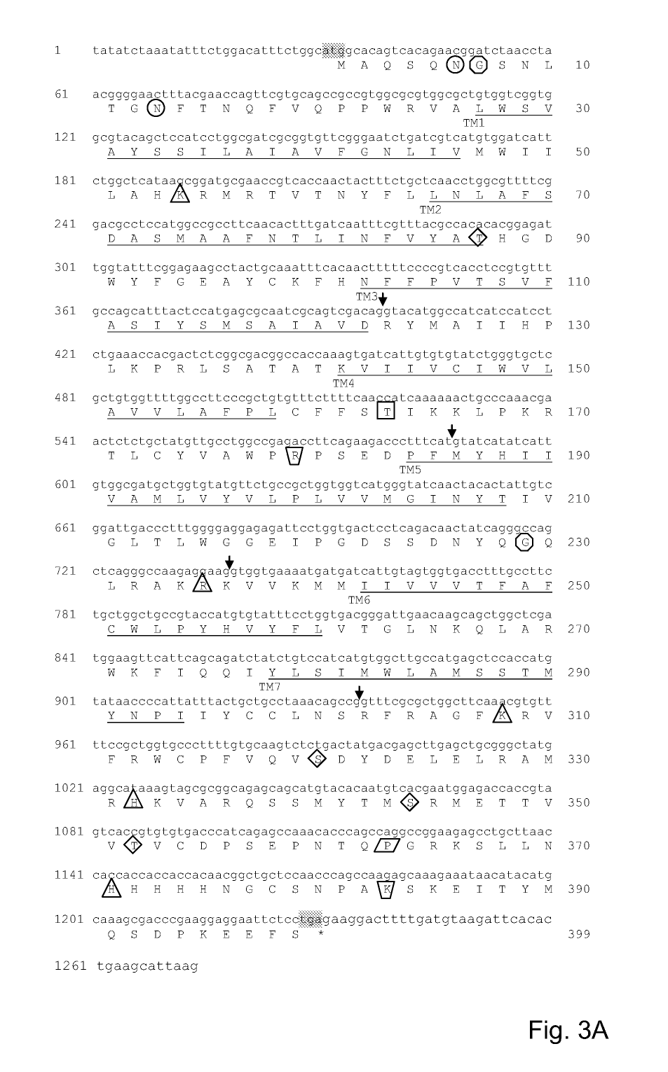

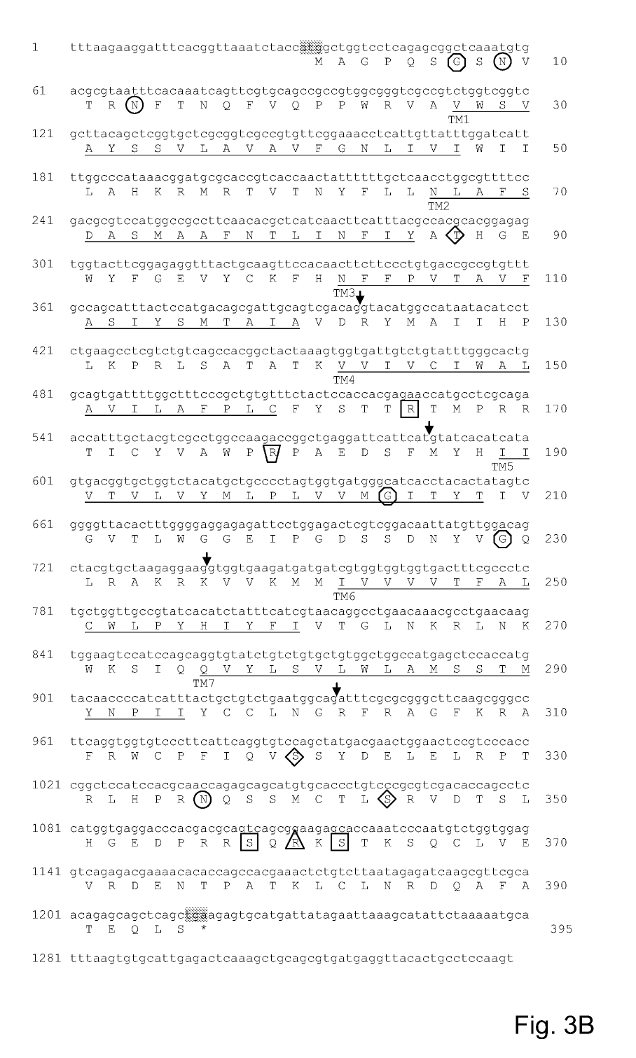

The full length receptors tac3ra and tac3rb cDNA (denoted by SEQ ID NO:6 and 7, respectively) from zebrafish brain were cloned by RT-PCR with specific primers, as listed in Table 1, above. As shown in FIG. 3, tac3ra cDNA has an ORF composed of 125 amino acids (FIG. 3A, GenBank accession No. JF317292), while tac3rb has an ORF of 115 aa (FIG. 3B, GenBank accession No. JF317293). The predicted tac3ra and tac3rb N termini have features consistent with a signal peptide, FIG. 3,). Sequence analysis of the two types of zebrafish receptors identified distinct potential sites for N-glycosylation, protein kinase C (PKC) phosphorylation, protein kinase A (PKA) phosphorylation, casein kinase II phosphorylation, tyrosine kinase phosphorylation, and N-myristoylation sites (see FIG. 3 and the legend thereof). The N- and C-terminals are, as for other G-protein coupled receptors, the most divergent regions. The homology between the different NKB receptors is shown in Table 4, below. Surprisingly, a third Tac3 receptor was found in sequence searches, so identification of more family members from different species was attempted, to allow confident assignment to the various Tac receptor family branches. Based on protein sequences encoding vertebrate NKB receptors, BLAST searches were performed to identify close homologues of the NKB receptor family in various species; in addition to mammalian NKB receptors that were annotated before, NKB receptors from different fish species were also identified by the inventors.

TABLE 4
Percent amino acid sequence identities (upper right to the table diagonal) and similarities (lower left
to the table diagonal) among Tac3r of different species as determined by EMBOSS* Stretcher alignment tool

| | Zebrafish | Zebrafish | Zebrafish | Human | Cow | Mouse | Chicken | Medaka | Medaka | Tetraodon | Tetraodon | Frog |
|---|---|---|---|---|---|---|---|---|---|---|---|---|
| | Tac3ra | Tac3rb | Tac3rc | Tac3r | Tac3r | Tac3r | Tac3r | Tac3ra | Tac3rb | Tac3ra | Tac3rb | Tac3r |
| Zebrafish TacR3A | — | 74.9 | 60.7 | 56.4 | 57.3 | 59.2 | 60.6 | 73.8 | 68.4 | 71.8 | 65.7 | 60 |
| (cypriniformes) | | | | | | | | | | | | |
| Zebrafish TacR3B | 85 | — | 61.3 | 57.5 | 57.7 | 59.7 | 62 | 74.5 | 72.7 | 73.3 | 71.1 | 59.4 |
| (cypriniformes) | | | | | | | | | | | | |
| Zebrafish TacR3C | 75.9 | 71.7 | — | 50.5 | 51.3 | 53.1 | 52.2 | 61.9 | 61.3 | 62.4 | 58.6 | 52.3 |
| (cyriniformes) | | | | | | | | | | | | |
| Human TacR3 | 67.6 | 67.3 | 63.8 | — | 90.1 | 86 | 75.3 | 59.3 | 54.2 | 57.6 | 52.5 | 68.8 |
| Cow TacR3 | 67.9 | 67.4 | 64.2 | 93.1 | — | 86 | 76.3 | 60.3 | 54 | 57.2 | 52.1 | 69.7 |
| Mouse TacR3 | 70.4 | 69.5 | 64.9 | 90.5 | 90.3 | — | 76.6 | 59.7 | 54.6 | 59.7 | 54 | 70.3 |
| Chicken TacR3 | 73.8 | 72.7 | 67.4 | 82.6 | 83.8 | 84.1 | — | 61.7 | 57.9 | 60.7 | 55.1 | 71.2 |
| Medaka Tac3RA | 84 | 84 | 71.4 | 69.5 | 70.2 | 71.7 | 74 | — | 70.4 | 83.3 | 67.2 | 61 |
| (beloniformes) | | | | | | | | | | | | |
| Medaka Tac3RB | 80.5 | 82 | 74.7 | 63.7 | 63.3 | 65 | 68.8 | 79.9 | — | 68.8 | 74.9 | 54.4 |
| (beloniformes) | | | | | | | | | | | | |
| Tetraodon Tac3RA | 82.5 | 82.3 | 75.5 | 68 | 68.3 | 70.4 | 73.5 | 92 | 77.2 | — | 66.7 | 57.4 |
| (tetraodontiformes) | | | | | | | | | | | | |
| Tetraodon Tac3RB | 75.2 | 79 | 72.3 | 62.2 | 61.3 | 63.3 | 64.5 | 75.7 | 82.7 | 74 | — | 53.2 |
| (tetraodontiformes) | | | | | | | | | | | | |
| Frog TacR3 | 73.8 | 72.4 | 68.3 | 78.6 | 78.2 | 80.4 | 83.4 | 75.9 | 67.8 | 73.6 | 66 | — |

— Same species, not applicable.

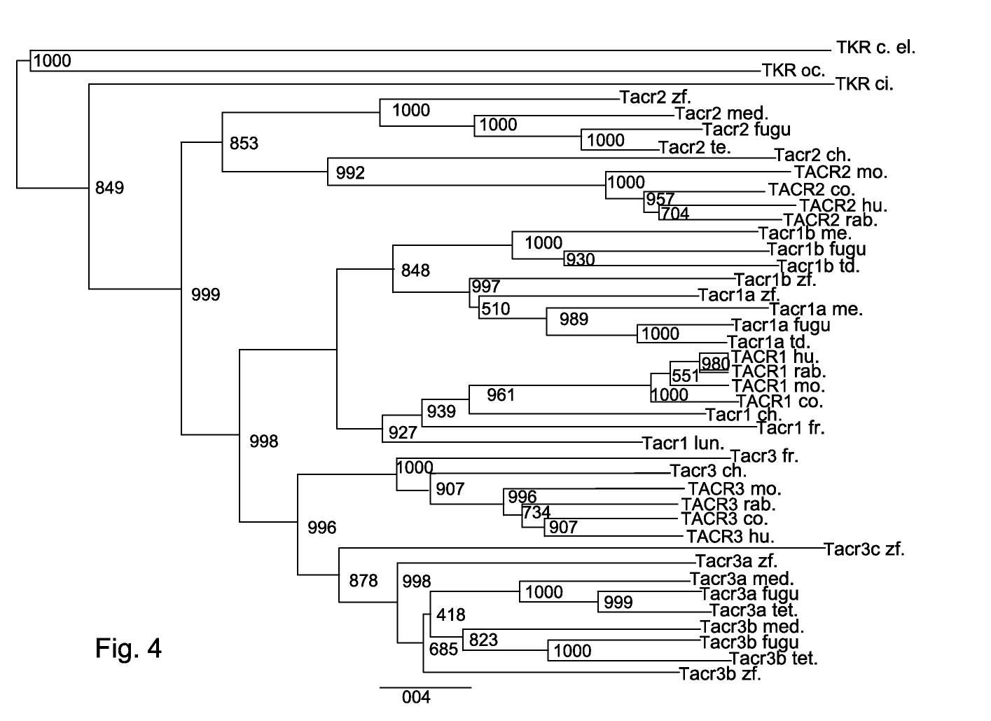

As graphically presented in FIG. 4, the phylogenetic tree contained vertebrate NKB receptors form three clearly separable groups that correspond to TAC3R, TAC1R and TAC2R. Putative orthologs of NKB receptors members were additionally identified in several nonvertebrate species, namely, c. elegans, ciona and octopus, that served as outgroup sequences and determined the root of these three groups. The root is located between TAC2R and TAC1R, indicating that these groups split early in evolution of the family. The tree shows that TAC3R and TAC3R are closest to each other, suggesting that their separation is a more recent evolutionary event.

Two forms of tac3 genes were found in zebrafish and salmon, but more evolved fish contained only one tac3 ortholog; however, all fish species exhibit two forms of NKB receptors, suggesting that the piscine NKB/NKBR can provide an excellent model for understanding the molecular coevolution of the peptide/receptor pairs.

### Example 3

### Chromosomal Synteny of tac3 and tac3 Receptor

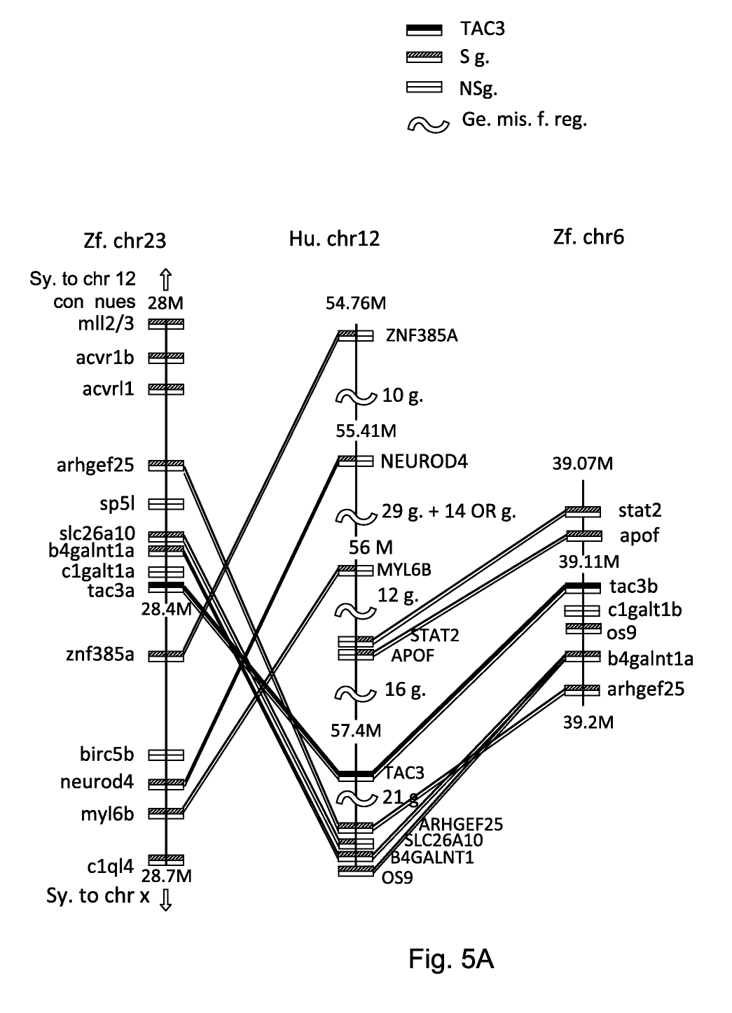

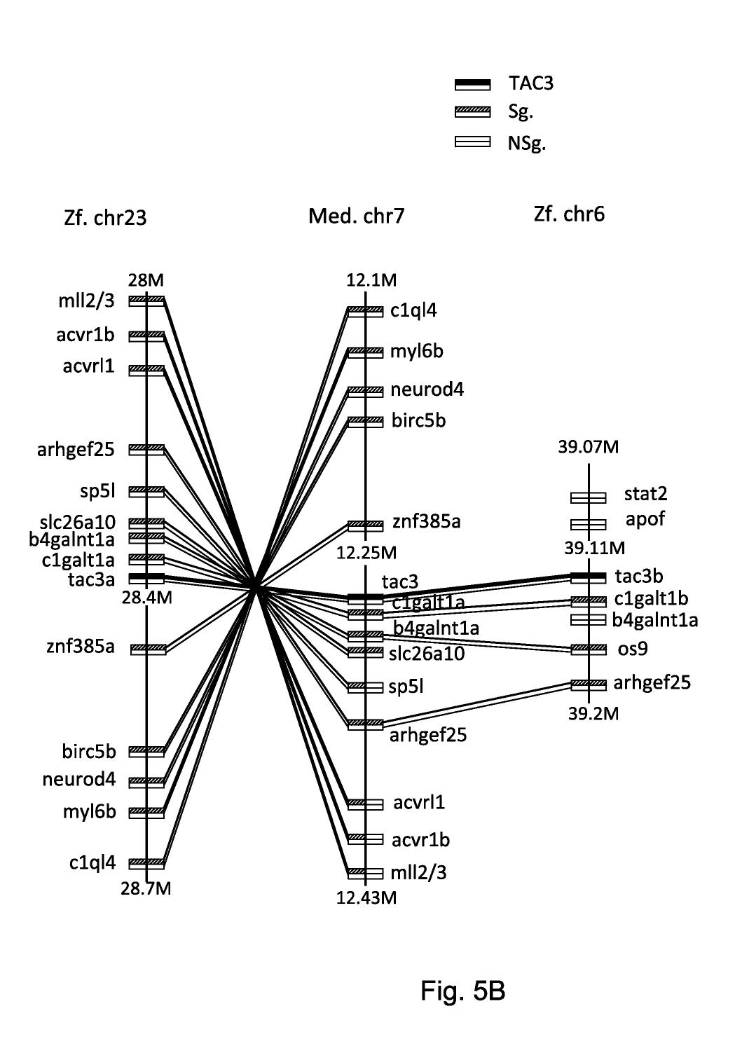

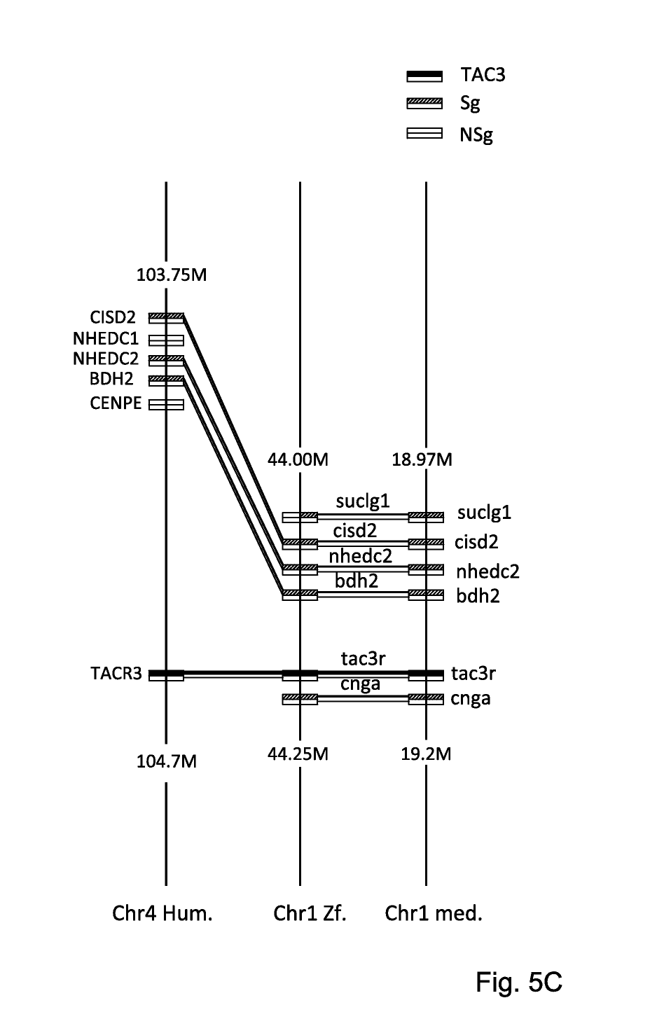

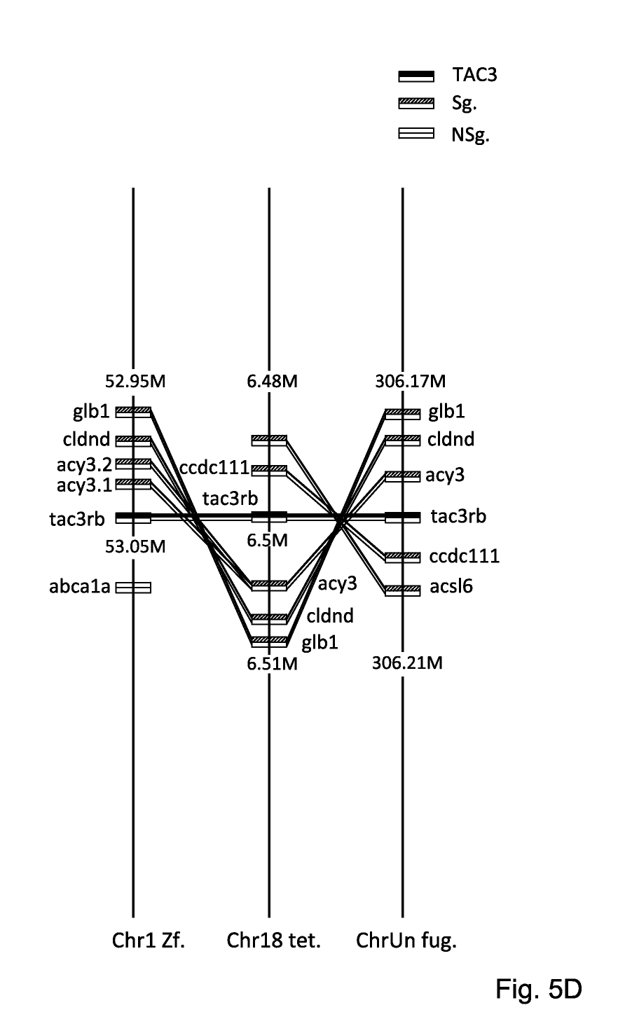

Chromosome syntenic analysis revealed that the locus of tac3 is highly conserved between teleosts (FIG. 5). Zebrafish tac3a is located on chromosome 23 while zebrafish tac3b is located on chromosome 6. The only tac3 found in medaka is located on chromosome 7 (see FIG. 5B). For the zebrafish tac3 gene, the first nearest neighboring gene (c1galt1a) is non-syntenic gene, while the next neighboring genes (b4galnt1a and slc6a1) were found in inverse order in the human (FIG. 5A). Despite nearly perfect preservation of synteny, a substantial shuffling of gene order along corresponding chromosome arms was found between the zebrafish and the human. The neighborhood of gene loci of tac3a, are conserved in the zebrafish, fugu, and medaka (FIG. 5B).

The genomic locations of tac3 receptors in human and in various fish species was then explored (FIGS. 5C and D). Human TAC3R is located on chromosome 4, while in zebrafish, fugu, madaka and Tetraodon, tac3ra are located on chromosome 1. For the zebrafish tac3ra gene, the first nearest neighboring gene (cnga2) is non-syntenic gene with the human, but synthetic with Medaka, fugu and Tetraodon. The next up-stream neighboring genes (bdh2, nhedc2, and cisd2) were found in similar locations in all species analyzed (FIG. 5C). Human genome lacks tac3rb, while in tetroadon, fugu and medaka, it exists (FIG. 5D). The next up-stream neighboring genes in the zebrafish (acy3.1, acy3.2, cldnd and glb1) were found in reverse order in the tetraodon (FIG. 5D).

### Example 4

### Tissue Distribution of tac3 and tac3 Receptors in Zebrafish

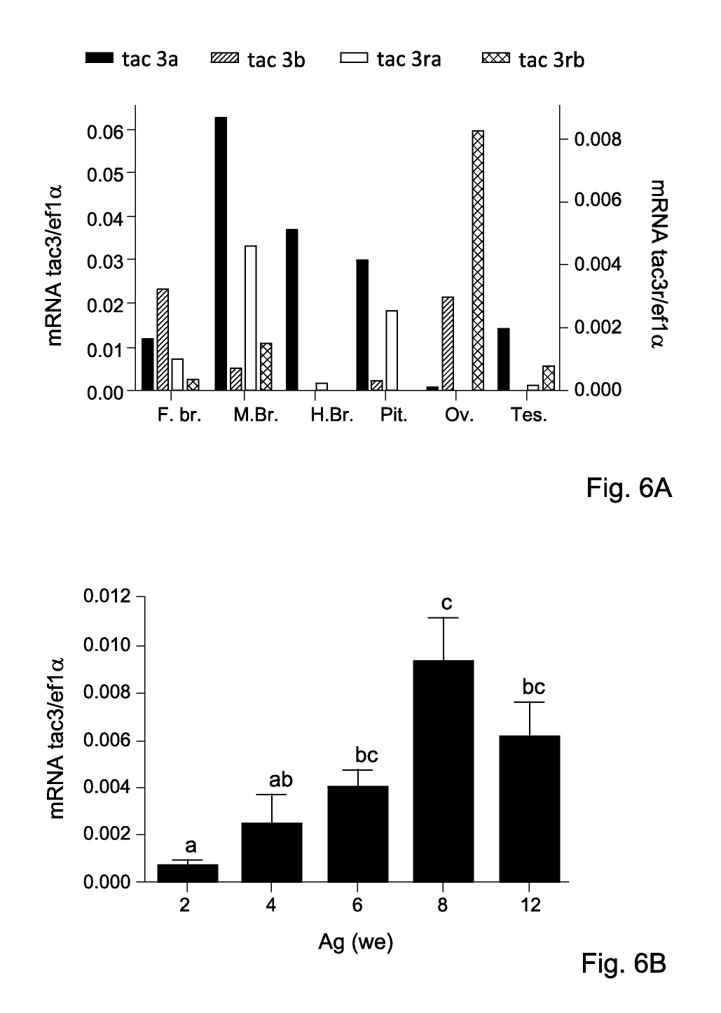

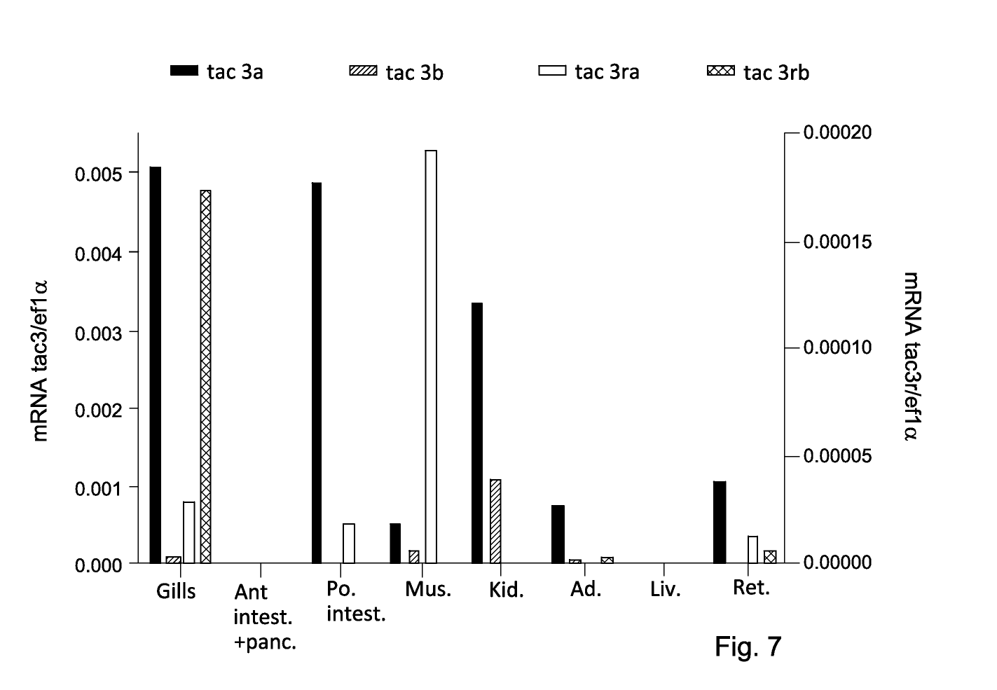

In order to elucidate the physiological role(s) of the NKB/NKBR signaling system, the tissue distribution of both ligands (tac3a, tac3b) and receptor (tac3ra, tac3rb) mRNAs in zebrafish was next examined by means of real-time PCR analysis, according to the publication by Biran et al., 2008 [4]. Briefly, the zebrafish brain was dissected into three parts, of which the anterior part contains the telencephalon, the midbrain contains the optic tectum, diencephalon, and hypothalamus, and the hindbrain the medulla oblongata and cerebellum. As demonstrated in FIG. 6A, tac3a mRNA was detected mostly in the midbrain, while tac3b mRNA was detected mainly in the forebrain. Both tac3a and tac3ra were expressed in the pituitary (FIG. 6A), corroborating with findings in mammals, where NKB and N3R were both expressed in the median eminence—which is missing in fish [2]. As also shown in FIG. 6A, tac3rb was expressed in the forebrain and was highest in the ovary. Different types of tac3 and tac3r were expressed in the ovary and testis (FIG. 6). Low levels of mRNA expression of all four transcripts were found in the liver, retina, adipose tissue. However, relatively high mRNA levels of tac3a and tac3rb were expressed in the gills, tac3a in the posterior intestine and tac3ra in the muscle (FIG. 7). The expression patterns of these genes in the brain-pituitary-gonad axis further support the potential role of the NKB system in fish reproduction.

### Example 5

### Gene Expression of NKB mRNA During Sexual Maturation

In order to decipher the involvement of the NKB/NKBR system in processes leading to puberty in fish, the expression profiles of zebrafish tac3a mRNA was evaluated in the brain during several different stages of development, by means of real-time PCR. As shown in FIG. 6B, low expression of tac3a mRNA was detected in zebrafish at the age of 2-4 week (wk) post fertilization. The expression levels of this gene gradually increased and peaked at 8 wk post fertilization. Zebrafish tac3a mRNA levels subsequently decreased at 12 wk post fertilization (FIG. 6B), when it is known that at this age, the fish gonads contain clear, well-developed oocytes and spermatozoa [4]. The increase of tac3a mRNA toward puberty, which is parallel to the increase in kiss1 at this age [6, 19] may point for a possible involvement of the tac3/tac3r system in puberty.

### Example 6

### Localization of Embryonic tac3a Cells by Whole Mount In Situ Hybridization

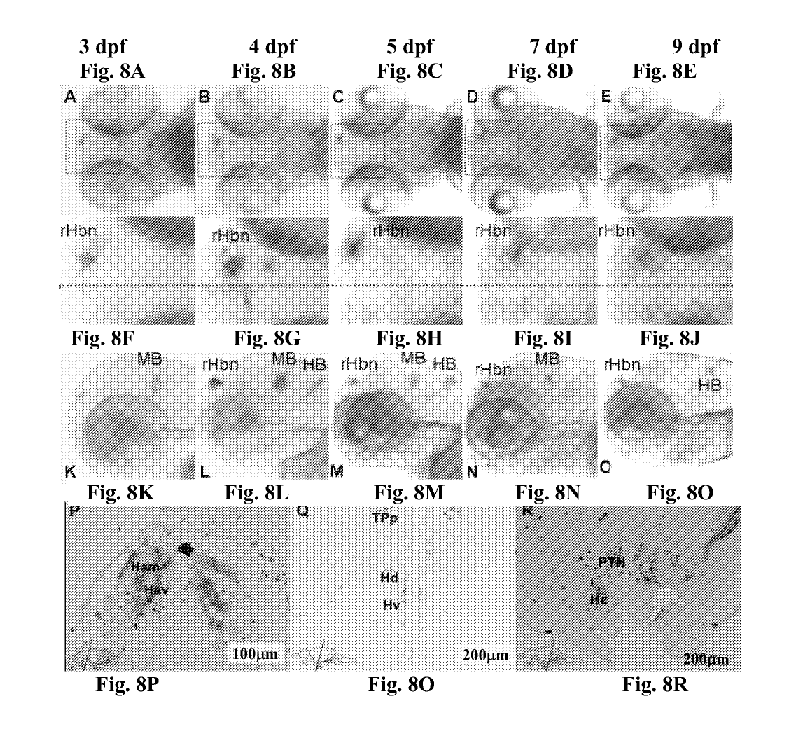

The first appearance of tac3a expression was detected 3 days post fertilization (dpf) at the right habenula nuclei and the midbrain (FIGS. 8A, F, K). At 4 to 5 dpf, the signal intensity at the habenula and midbrain increased, probably reflecting increased cell number (FIGS. 8B, C, G, H, L and M). In addition, expression at the hindbrain was noted. Analysis of elderly larvae (7 and 9 dpf) revealed a decrease in tac3a signal intensity (FIGS. 8D, E, I, J, N, O), and at 12 dpf was barely detected.

Localization of tac3a expression during early stages of development as detected by whole mount in situ hybridization revealed a specific and robust signaling at the habenula, midbrain and hindbrain (FIG. 8). The unilateral expression of tac3a to right of the midline (dotted line) in the habenula (F-J) is of note. During embryogenesis tac3a is dominantly expressed at the right habenula nuclei, whereas at adulthood, tac3a is expressed at both of the habenula lobes (FIG. 8 P). This asymmetrical expression of tac3a in the habenula is consistent with previous results shown that the habenula in zebrafish display left-right asymmetries in gene expression [20, 21]. Interestingly, it was shown that neurons in the left habenula are present earlier than in the right [22]. Neuronal organization asymmetries in the epithalamus (i.e. habenular nuclei and pineal complex) are well-known amongst vertebrates [23]. Therefore, zebrafish tac3a can be used as a useful marker for the research of vertebrate brain lateralization.

### Example 7

### Localization of tac3a mRNA in the Brain of Adult Zebrafish

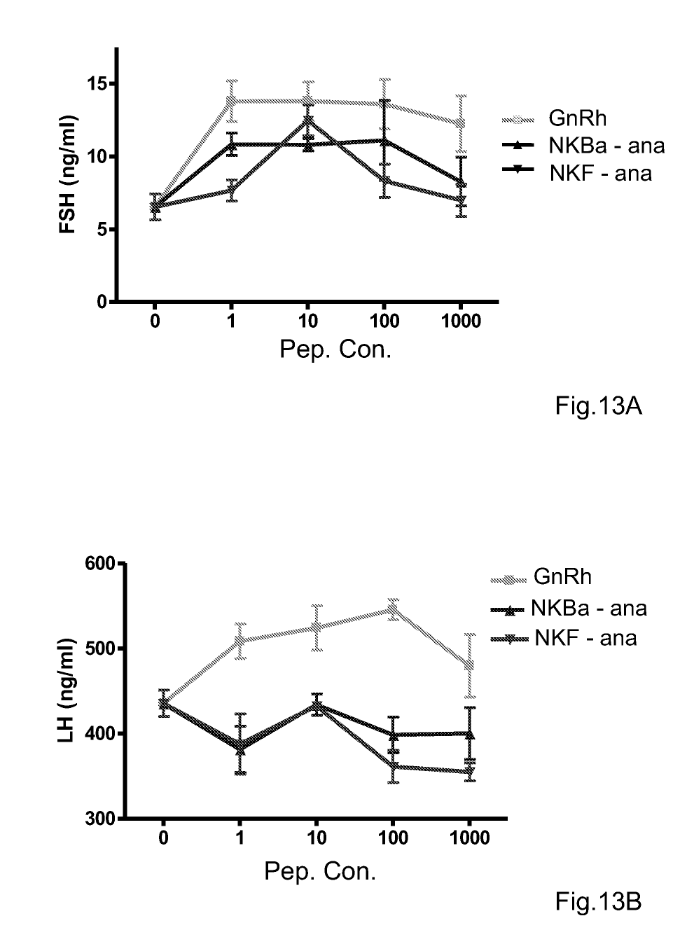

Localization of tac3a mRNA in the zebrafish brain was also determined by means of in situ hybridization (ISH) techniques. As shown in FIGS. 8 P, Q and R and as noted above, tac3a mRNA expressing neurons were detected in the Habenula (Ha; FIG. 8P), along periventricular Hypothalamus (FIGS. 8 Q and R), the periventricular nucleus of posterior tuberculum (TPp; FIG. 13C), and the posterior tuberal nucleus (PTN; FIG. 8R). These brain nuclei were previously shown to express other important neuropeptides that regulate reproduction [19], metabolism [24], and stress [25]. In mammalian, arcuate nucleus (ARC), KISS1 neurons that co-express NKB and dynorphin were proposed as a central node into which potential stress, metabolic and photoperiodic signals are conveyed to regulate GnRH release [26]. The nucleus lateralis tuberis (NLT) is considered as the piscine homologous structure to the mammalian ARC [24]. Without wishing to be bound by theory, the localization of tac3a, kiss2, kiss1rb, lepr, MCH2 and two MCH1Rs, Urotensin I, CRF and CRF-binding protein to the ventral zone of the periventricular hypothalamus, as presently demonstrated in FIG. 8Q for tac3a and was previously shown for the other cellular components mentioned above by others [27, 32 and 32] suggests that not all neuropeptide pathways are as conserved as formerly thought.

### Example 8

### Pharmacological Analysis and Signal Transduction Pathways of zftac3

The response, binding selectivity and signal transduction pathways of the novel tachykinin receptors to their agonists were evaluated by functional expression analysis, using COS-7 cells. Graded concentrations of the novel zebrafish tachykinins peptides (NKBa, NKBb and NKF), as well as human NKB (hNKB) and its agonist senktide were applied to COS-7 cells that express the human NKB receptor (huNKBR), Tac3ra or Tac3rb. To perform the functional expression analysis, the reporters SRE-Luc and CRE-Luc were used, the specificity of which to the activation of PKC/Ca²⁺ and PKA/cAMP signal transduction pathways, respectively were demonstrated before by the inventors [4]. The EC₅₀ values of tachykinins for each receptor are summarized in Table 5, below.

TABLE 5
EC₅₀ values (nM) of human and unique piscine NKBs

| CRE or | | | |
|---|---|---|---|
| SRE NKBR | zfTac3ra | zfTac3rb | huNK3R |
| NKBRs EC₅₀s | | | |
| CRE-Luc (nM) | | | |
| zfNKBa | 5.75 ± 1.45 | 4.55 ± 1.57 | 3.73 ± 1.25 |
| zfNKBb | 237.20 ± 134.20 | 519.20 ± 160.30 | 605.00 ± 132.00 |
| zfNKF | 4.94 ± 1.83 | 1.80 ± 1.55 | 4.36 ± 1.25 |
| huNKB | 12.94 ± 13.3 | 8.12 ± 1.56 | 4.71 ± 2.08 |
| Senktide | 48.95 ± 14.89 | 20.10 ± 15.1 | 17.41 ± 1.26 |
| NKBRs EC₅₀s | | | |
| SRE-Luc (nM) | | | |
| zfNKBa | 0.50 ± 0.17 | 1.47 ± 1.66 | 0.49 ± 0.18 |
| zfNKBb | 8.96 ± 1.19 | 33.83 ± 14.7 | 204.40 ± 138.60 |
| zfNKF | 0.54 ± 0.13 | 0.36 ± 0.16 | 0.52 ± 0.18 |
| huNKB | 2.20 ± 1.49 | 0.82 ± 0.16 | 0.67 ± 0.16 |
| Senktide | 2.67 ± 1.44 | 2.72 ± 1.54 | 1.51 ± 1.63 |

Serum responsive element (SRE)-Luc was used as a reporter gene that follows PKC activation;
cAMP responsive element (CRE)-Luc was used to follow PKA activation.
Mean ± SEM

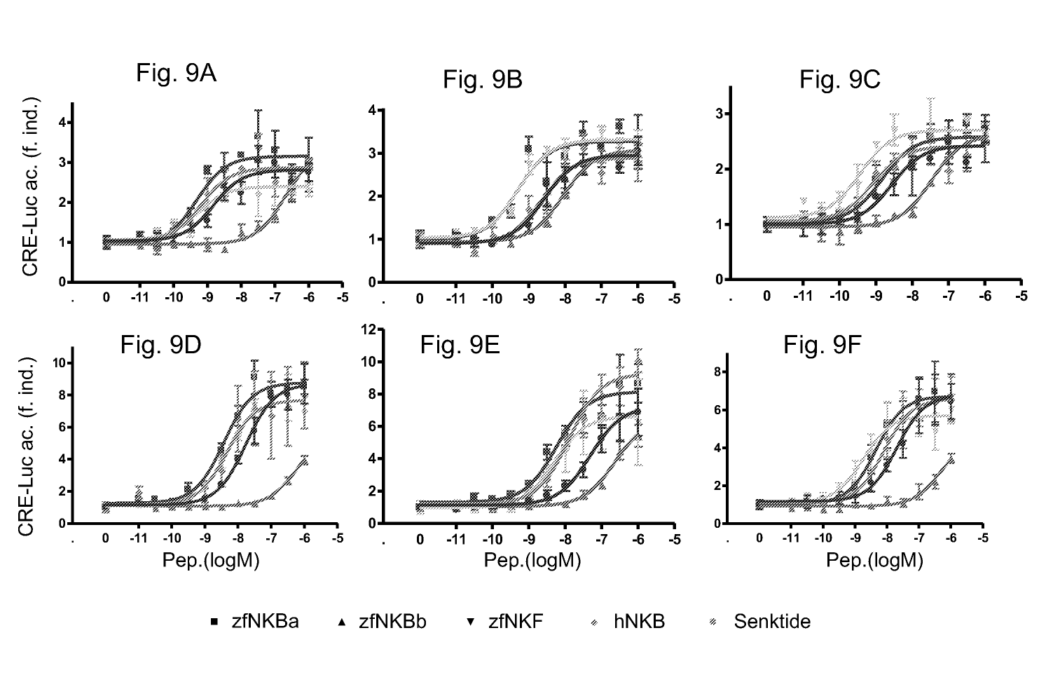

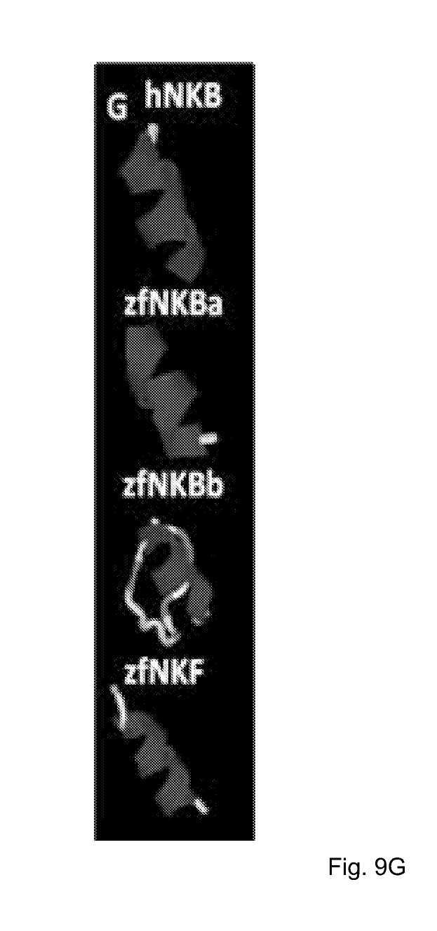

As demonstrated in FIG. 9, both human and piscine tachykinins induced a concentration-dependent increase in both SRE-Luc and CRE-Luc activity. For human NKBR (huNK3R), in both signal transduction systems, zebrafish NKBa and NKF showed high potency, similar to the potency shown by human NKB, however, zebrafish NKBb peptide exhibited a relatively low potency (FIGS. 9A, D). A similar pattern was demonstrated for both zebrafish tac3 receptors, where zebrafish NKBa (i.e. the peptide derived from tac3a), and zebrafish NKF (also derived from tac3a), were the most potent among all tachykinins examined, in both signal transduction systems (FIG. 9). These results revealed that both novel peptides (zebrafish Tac3a and NKF) are all endogenous ligands of Tac3 receptors. It is noteworthy that this is the first report of a tachykinin receptors that are shown to be activated by a second peptide derived from the NKB gene (zebrafish NKF). However, NKBb was less effective than the other forms in eliciting luciferase activity by both signal transduction pathways. The results thus demonstrate that the zebrafish novel receptors disclosed herein relay their signal trough both protein kinase C (PKC) and protein kinase A (PKA) transduction pathways, as manifested by the SRE-Luc and CRE-Luc activity assays.

### Example 9

### Ligand Models

FIG. 9G provides a ribbon representation of the zebrafish Tac3 crystal structures in comparison to the human hNKB (PDB ID 1p9f). Intriguingly, although the three zfNKBs vary in size 10, 24, and 13 aa for NKBa, NKBb, and NKFa, respectively, all of the predicted peptides yielded high-resolution, high-quality structures with typical globular folding consisting of alpha-helix-loop motifs (FIG. 9G). Thus, all three zebrafish Tac3 crystal structures approximate a binding competent conformation similar to that of the human NKB. Mammalian NKB forms a helical structure in the presence of dodecylphosphocholine micelles [27]. Without wishing to be bound by theory, the overall induction of a helical conformation in the mid region of each of the tachykinins appears to be crucial for tachykinin receptor activation. Selectivity for each receptor has been attributed to changes in the helix length having an effect on the distribution of the hydrophobic and hydrophilic extremes of the tachykinin peptides.

### Example 10

### In Vivo Effect of Estradiol

The inventors next examined the involvement of the NKB system in reproduction. In fish, clear evidence exists regarding the important role played by estradiol during the period of reproduction [3]. Zebrafish have two forms of gonadotropin-releasing hormone: GnRH2 is localized to the midbrain tegmentum, and GnRH3 (considered to be the hypophysiotropic form) is located at both the olfactory bulb terminal nerve (OB-TN) and the preoptic area (POA) [28]. Since in mammals, kisspeptin and NKB are expressed in the same neurons in the hypothalamic arcuate nucleus (ARC), and play a key role in physiological regulation of GnRH neurons [26], the effect of estradiol on the expression of genes along the GnRH-kisspeptin system was tested. As shown in FIG. 10A, estradiol treatment of juvenile (prepubertal) zebrafish enhanced expression of key genes involved in reproduction (gnrh3, kiss2 and kiss1) concomitantly with a significant increase in the expression level of tac3a. In parallel a significant increase in the expression of tac3ra, tac3rb and kiss1ra was detected (see FIG. 10B). As shown above, both Tac3 receptors bound the novel zebrafish NKBs (FIGS. 9B-C and E-F), and kiss1ra was previously shown to bind kiss2 with higher affinity than kiss1 [29]. In mammals, there is strong evidence of sexual dimorphism of NKB neurons: larger numbers of NKB neurons have been identified in ARC of ewes than of rams [30]. Without wishing to be bound by theory, the transcription of NKB could be directly altered by estrogen receptors, as sequences corresponding to the estrogen responsive element and imperfect palindromic ERE have been reported upstream of the TAC3 gene transcriptional start site [7]. In fish estradiol is involved in both early oogenesis and the beginning of the first wave of vi-tellogenesis that precede puberty, collaborating these findings that tac3a expression peaked in prepubertal fish (FIG. 6A). Moreover, increased levels of estradiol are a required characteristic of both follicular growth and final oocyte maturation in fish, pointing toward the involvement of the piscine NKB system in control of reproduction, probably in concert with kisspeptin and GnRH.

### Example 11

### In Vivo Effect of NKBs in Zebrafish

To further characterize the role of the NKB/NKBR system in fish reproduction the in vivo biological function of zebrafish NKB peptides was next examined. A single intraperitoneal injection of zfNKBa or zfNKF elicited a significant LH secretion in sexually mature female zebrafish (FIG. 10C). The magnitude of the induced LH discharge was comparable with that observed in response to GnRH. LH response to the hNKBR agonist, senktide, or zfNKBb was less pronounced (FIG. 10C), in a similar way to the order of potency obtained in the transactivation assay (FIG. 9).

### Example 12

### In Vivo Effect of NKBs Analogues

The activation of NKB receptors was then tested by their cognate novel fish NKBs as well as by analogues (agonists) thereof.

The NKB analogues (i.e. the NKBa-analog, NKBb-analog and NKF-analog, as denoted by SEQ ID NO. 44-46) were first analyzed in comparison to their native forms in a transactivation assay, using reporter genes for specific signal transduction pathways. To validate the specificity of the serum-responsive element (SRE)-Luc and cAMP-responsive element (CRE)-Luc reporter systems, specific activators for PLC/PKC and AC/PKA signaling pathways were used in control experiments. As described in Biran et al., 2008 [4], the SRE-Luc reporter system was significantly activated by a PKC activator, but not by the PKA activator, whereas the CRE-Luc reporter system was activated by PKA activator but not by the PKC activator.

Thus, the ability of the three novel piscine peptides (namely, NKBa, NKBb and NKF, denoted by SEQ ID NO.40, 41 and 42), as well as the ability of the three analogous agonistic peptides, namely, NKBa-analog, NKBb-analog and NKF-analog, to differentially activate the different tac3 receptors, using SRE as a reporter gene that follows PKC activation and CRE as a reporter gene for the PKA pathway, was tested. Human NK3R (human NKB receptor) and human NKB served as controls.

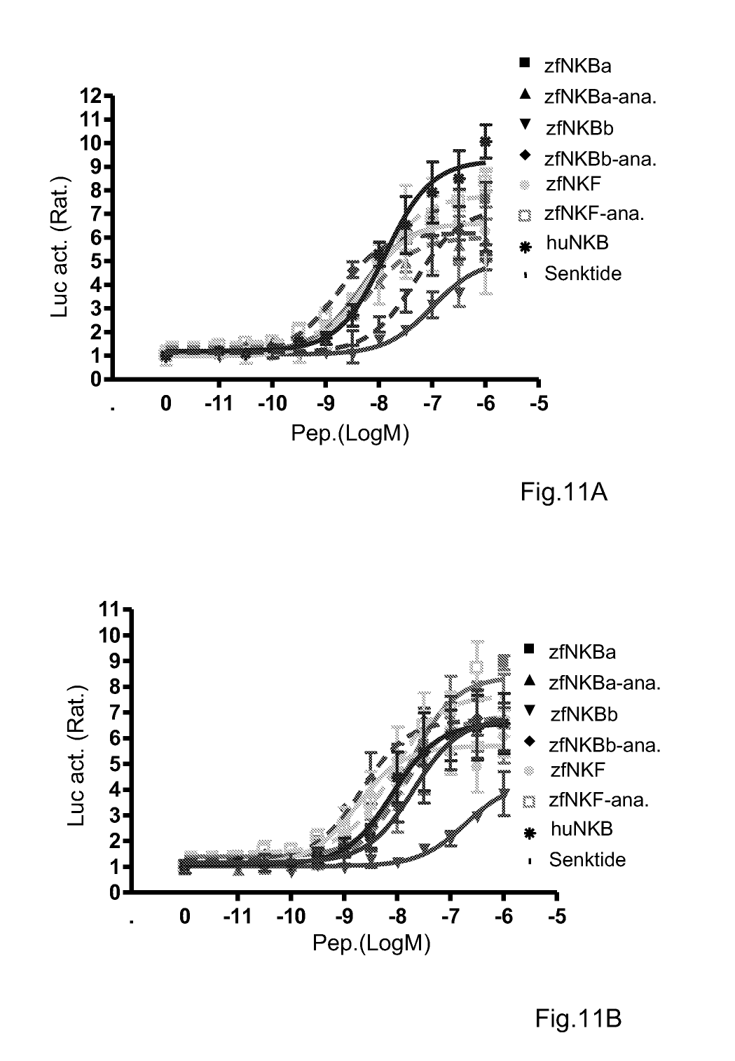

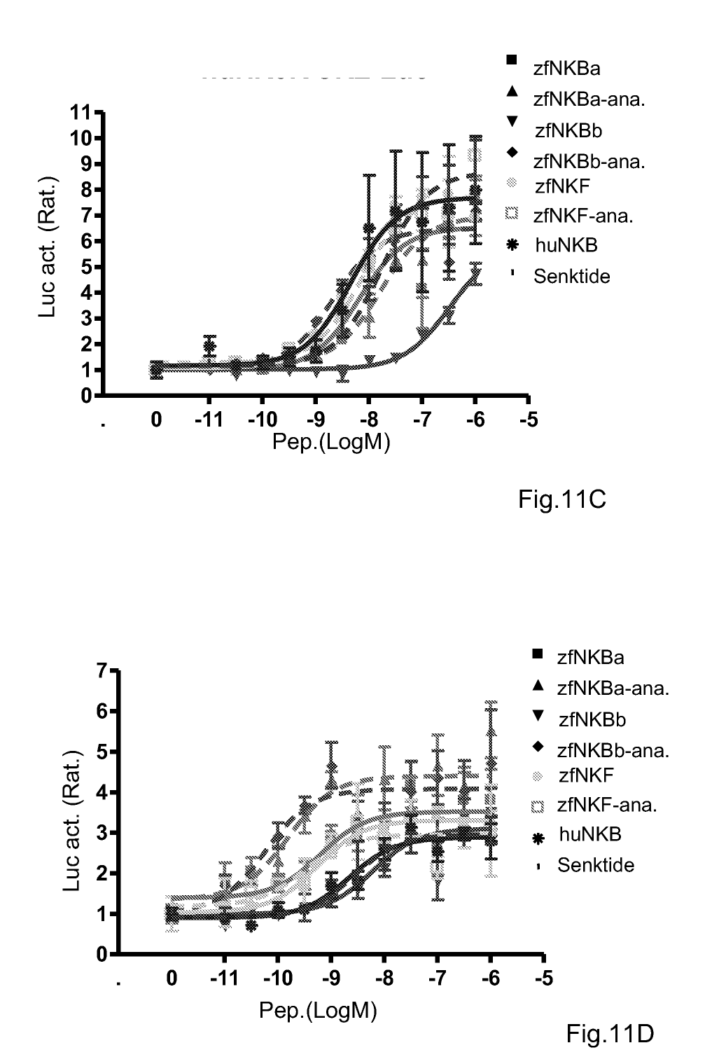

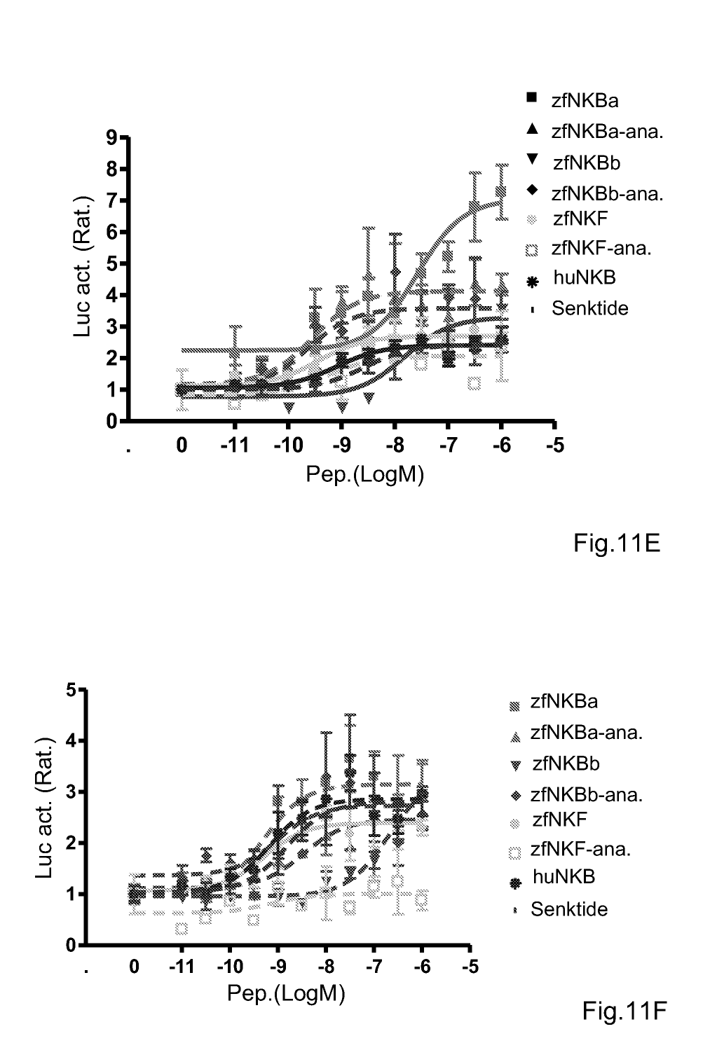

As monitored by a reporter assay based on measuring SRE-driven luciferase activity in COS-7 cells, transiently transfected with human NK3R, all three piscine NKBs (NKBa, NKBb and NKF) elicited an increased response, when NKBb has the lowest effect (FIG. 11F). NKBb was found to have the lowest effect also when it was tested on activation of the piscine NKB receptors (FIGS. 11A, B, D, E). The most effective peptide was NKBa. All the new analogues were more effective than their native forms, meaning lower EC₅₀ values, implicating that lower concentrations of the NKB analogs were required. The most pronounced effect, at the PKA pathway, was observed for the peptides NKBb and its analogue, on Tac3rb (EC₅₀=191.1 vs. 2.2 (zfTac3rb), or 99.6 vs 1.7 (zfTac3ra) for NKBb and NKBb-analog, respectively; FIG. 11B and Table 6, below) the effect of NKF on Tac3rb (EC₅₀=1.8 vs. 7.75, for NKF and NKF-analog, respectively; FIG. 11B; Table 6); the effect of NKBa on Tac3rb (EC₅₀=16 vs. 11.9, for NKBa and NKBa-analog, respectively; FIG. 11A; Table 5). The most efficient increase, at the PKC pathway, was found with the effect of NKBb on Tac3ra (EC₅₀=6.06 vs 0.06 for NKBb and NKBb-analog, respectively; FIG. 11D; Table 7); the effect of NKF on Tac3ra (EC₅₀=0.55 vs. 0.31, for NKF and NKF-analog, respectively; FIG. 11D; Table 7); the effect of NKBa on Tac3rb (EC₅₀=26.06 vs. 0.29, for NKBa and NKBa-analog, respectively; FIG. 11E; Table 7); the effect of NKBb on Tac3rb (EC₅₀=15.3 vs. 0.23, for NKBb and NKBb-analog, respectively; FIG. 11E; Table 7).

TABLE 6
EC₅₀ values (nM) of human and novel piscine NKBs, and their respective
analogues, using CRE-Luc to follow PKA activation. Mean ± SEM.

| | zfTac3ra | zfTac3rb | huTac3R |
|---|---|---|---|
| zfNKBa | 4.75 ± 1.48 | 16.43 ± 11.98 | 6.64 ± 1.53 |
| zfNKBa-analog | 6.94 ± 1.28 | 11.87 ± 16.94 | 16.78 ± 14.44 |
| zfNKBb | 99.56 ± 12.94 | 191.1 ± 154.67 | 336.40 ± 133.10 |
| zfNKBb-analog | 1.68 ± 1.21 | 2.18 ± 1.34 | 2.19 ± 1.33 |
| zfNKF | 4.94 ± 1.83 | 1.8 ± 1.55 | 4.36 ± 1.25 |
| zfNKF-analog | 9.14 ± 1.27 | 7.75 ± 1.41 | 7.33 ± 1.30 |
| huNKB | 12.94 ± 13.3 | 8.12 ± 1.56 | 4.71 ± 2.08 |
| Senktide | 48.95 ± 14.89 | 20.1 ± 15.1 | 17.41 ± 1.26 |

TABLE 7
EC₅₀ values (nM) of human and novel piscine NKBs, and their respective
analogues, using SRE was as a reporter gene that follows PKC activation.
Mean ± SEM.

| | zfTac3ra | zfTac3rb | huTac3R |
|---|---|---|---|
| zfNKBa | 0.62 ± 0.22 | 26.06 ± 35.16 | 0.49 ± 0.18 |
| zfNKBa-analog | 0.15 ± 0.19 | 0.29 ± 0.23 | 3.44 ± 2.69 |
| zfNKBb | 6.06 ± 1.90 | 15.33 ± 18.26 | 204.40 ± 138.60 |
| zfNKBb-analog | 0.06 ± 0.03 | 0.23 ± 0.25 | 0.94 ± 0.44 |
| zfNKF | 0.55 ± 0.13 | 0.37 ± 0.16 | 0.52 ± 0.18 |
| zfNKF-analog | 0.31 ± 0.26 | 0.54 ± 0.39 | 0.71 ± 1.40 |
| huNKB | 2.20 ± 1.49 | 0.82 ± 0.16 | 0.67 ± 0.16 |
| Senktide | 2.68 ± 1.44 | 2.72 ± 1.54 | 1.50 ± 1.63 |

### Example 13

### NKB Analogues Increase Gonadotropin Release In-Vivo from Juvenile Tilapia

Tilapia females (two-months old) were injected with the NKBa analogue denoted by SEQ ID NO.44 or the NKF analogue denoted by SEQ ID NO.46 at 0.5 or 5 μg/kg body weight (BW). Blood was withdrawn from the caudal vein and plasma Luteinizing hormone (LH) and Follicle-stimulating hormone (FSH) levels were measured using a homologous sensitive enzyme-linked immunosorbent assay (ELISA), as described by Aizen et al. 2007 [33]. As shown in FIG. 12A, the NKF analogue (at 5 μg/kg) increased significantly the level of FSH. A similar effect was demonstrated by the NKF analogue (at 5 μg/kg), in increasing the level of LH (FIG. 12B).

### Example 14

### NKB Analogues Increase Gonadotropin Release from Tilapia Pituitary Dispersed Cells

Tilapia pituitary cells were subjected to enzymatic dispersion. Pituitary cells were stimulated with 10 nM of the NKBa or NKF analogue, denoted by SEQ ID NO.44 and SEQ ID NO.46, respectively, for 4 hr, when the medium was collected. As demonstrated in FIG. 13A, both analogues significantly increased the release of FSH from tilapia pituitary cells, indicating that the pituitary contains receptors for neurokinin B, as was also shown in the tissue distribution of the tac receptors. Stimulation of tilapia pituitary cells with GnRH at [10 nM] was used as a control.

### Example 15

### NKB Analogues Increase Gonadotropin Release from Mature Carp, In-Vivo

Mature female carp, just before spawning, were injected with NKBa, NKBb and NKF analogues, denoted by SEQ ID NO.44, SEQ ID NO.45 and SEQ ID NO.46, respectively (20 μg/kg body weight). LH levels was measured by specific ELISA, according to Aizen et al. 2012 [33]. As demonstrated in FIG. 14, all the three NKB analogues increased the level of LH significantly, although the effect remained constant throughout the experiment only for the NKF analogue.

### Example 16

### Development and Evaluation of Strategies to Advance the Onset of Puberty with NKB

The effect of NKB on gonadal development is examined by applying the most effective dose, at the most effective mode and way of administration of the NKB analog. These hormonal manipulations are then applied to fish, which are considered as too young to breed. The steroids (E2 and 11KT) as well as the gonadotropins (LH and FSH) hormonal levels in these fish are then analyzed in parallel, and a histological examination of the fish gonads is performed.

### Example 17

### The Effect of NKB Analogs on Ovulation and Final Oocyte Maturation

A group of mature fish (broodstock) is anaesthetized. Females are cannulated with a plastic catheter, and only females showing oocytes with migrating germinal vesicle are selected for injection. The migration of the germinal vesicle is checked using the SERRA solution (ethyl alcohol 96%: formalin: glacial acetic acid, 6:3:1, v/v) in a subsample of eggs under the stereoscope. Females are injected with NKB analogs at various doses and males are injected with the same doses. Several hours later, females are injected with a second, similar dose. Thereafter, the females are housed with the males. Next (24-72 hours later), gametes are collected by abdominal massage (stripping). The quality of the gametes is examined and eggs are collected by gentle abdominal massage pressure into a 500 ml glass vial. Eggs are placed onto a dried tray and then 0.4 ml sperm per each 100 ml eggs is added. Sperm is activated by thorough mixing. The time of sperm activation is taken as time zero. The eggs are then incubated in the incubation system until hatching.

TABLE 8
Sequence ID NO. used in the sequence listing

| SEQ ID | |
|---|---|
| NO | Description |
| 1 | Amino acid sequence of mouse Tac2, NP_033338.2 |
| 2 | Amino acid sequence of monodelphis domesitca, XP_001365310 |
| 3 | Amino acid sequence of ornAna1, Contig39139: 6403-6445 |
| 4 | Nucleic acid sequence of zebra fish full length tac3a (zftac3a), JN392856 |
| 5 | Nucleic acid sequence of zebra fish full length tac3b (zftac3b), JN392857 |
| 6 | Nucleic acid sequence of zebra fish full length tac3ra (zftac3ra), JF317292 |
| 7 | Nucleic acid sequence of zebra fish full length tac3rb (zftac3rb), JF317293 |
| 8 | Nucleic acid sequence for quantitative real time PCR, zf ef1a-1237F |
| 9 | Nucleic acid sequence for quantitative real time PCR, zf ef1a-1419R |
| 10 | Nucleic acid sequence for quantitative real time PCR, GnRH2-36F |
| 11 | Nucleic acid sequence for quantitative real time PCR, GnRH2-196R |
| 12 | Nucleic acid sequence for quantitative real time PCR, GnRH3-47F |
| 13 | Nucleic acid sequence for quantitative real time PCR, GnRH3-208R |
| 14 | Nucleic acid sequence for quantitative real time PCR, kiss1-10F |
| 15 | Nucleic acid sequence for quantitative real time PCR, kiss1-210R |
| 16 | Nucleic acid sequence for quantitative real time PCR, kiss2-137F |
| 17 | Nucleic acid sequence for quantitative real time PCR, kiss2-317R |
| 18 | Nucleic acid sequence for quantitative real time PCR, kiss1ra-856F |
| 19 | Nucleic acid sequence for quantitative real time PCR, kiss1ra-1095R |
| 20 | Nucleic acid sequence for quantitative real time PCR, kiss1rb-755F |
| 21 | Nucleic acid sequence for quantitative real time PCR, kiss1rb-1041R |
| 22 | Nucleic acid sequence for quantitative real time PCR, zf tac3a-F29 |
| 23 | Nucleic acid sequence for quantitative real time PCR, zf tac3a-R191 |
| 24 | Nucleic acid sequence for quantitative real time PCR, zf tac3b-F86 |
| 25 | Nucleic acid sequence for quantitative real time PCR, zf tac3b-R246 |
| 26 | Nucleic acid sequence for quantitative real time PCR, zf tac3ra F154 |
| 27 | Nucleic acid sequence for quantitative real time PCR, zf tac3ra R334 |
| 28 | Nucleic acid sequence for quantitative real time PCR, zf tac3rb F343 |
| 29 | Nucleic acid sequence for quantitative real time PCR, zf tac3rb R523 |
| 30 | Nucleic acid sequence for in situ hybridization or cloning, zf tac3a F-122 |
| 31 | Nucleic acid sequence for in situ hybridization or cloning, zf tac3a R360 |
| 32 | Nucleic acid sequence for in situ hybridization or cloning, zf tac3b F-17 |
| 33 | Nucleic acid sequence for in situ hybridization or cloning zf tac3b stop |
| 34 | Nucleic acid sequence for cloning, zf tac3Ra start |
| 35 | Nucleic acid sequence for cloning, zf tac3Ra stop |
| 36 | Nucleic acid sequence for cloning, zf tac3Rb start |
| 37 | Nucleic acid sequence for cloning, zf tac3Rb stop |
| 38 | Nucleic acid sequence of zebra fish tac3a (position −122 to 380 of |
| | JN392856) |
| 39 | Nucleic acid sequence of zebra fish tac3a (position 122 to 360 of JN392856) |
| 40 | Amino acid sequence of zebra fish Tac3a second fragment peptide (NKBa) |
| 41 | Amino acid sequence of zebra fish Tac3b second fragment peptide (NKBb) |
| 42 | Amino acid sequence of zebra fish first fragment peptide Tac3af (NKFa) |
| 43 | Amino acid sequence of zebra fish first fragment peptide Tac3bf (NKFb) |
| 44 | Amino acid sequence of NKBa-analog |
| 45 | Amino acid sequence of NKBb-analog |
| 46 | Amino acid sequence of NKF-analog |
| 47 | Amino acid sequence of Senktide (NKB agonist) |
| 48 | Amino acid sequence of mammalian NKB |
| 49 | Amino acid sequence of zebra fish full length tac3a |
| 50 | Amino acid sequence of zebra fish full length tac3b |
| 51 | Nucleic acid sequence of fathead minnow (Pimephales promelas) tac3, |
| | BK008100 |
| 52 | Amino acid sequence of fathead minnow (Pimephales promelas) tac3 |
| 53 | Amino acid sequence of fathead minnow (Pimephales promelas) tac3 first |
| | fragment |
| 54 | Amino acid sequence of fathead minnow (Pimephales promelas) tac3 second |
| | fragment |
| 55 | Nucleic acid sequence of channel catfish (Ictalurus punctatus) tac3, |
| | BK008101 |
| 56 | Amino acid sequence of channel catfish (Ictalurus punctatus) tac3 |
| 57 | Amino acid sequence of channel catfish (Ictalurus punctatus) tac3 first |
| | fragment (NKF) |
| 58 | Amino acid sequence of channel catfish (Ictalurus punctatus) tac3, second |
| | fragment (NKB) |
| 59 | Nucleic acid sequence of Atlantic salmon (Salmo salar) tac3a, BK008102 |
| 60 | Amino acid sequence of Atlantic salmon (Salmo salar) tac3a |
| 61 | Amino acid sequence of Atlantic salmon (Salmo salar) tac3a, first fragment |
| | (NKF) |
| 62 | Amino acid sequence of Atlantic salmon (Salmo salar) tac3a, second |
| | fragment (NKBa) |
| 63 | Nucleic acid sequence of Atlantic salmon tac3b, BK008103 |
| 64 | Amino acid sequence of Atlantic salmon (Salmo salar) tac3b |
| 65 | Amino acid sequence of Atlantic salmon (Salmo salar) tac3b, first fragment |
| | (NKF) |
| 66 | Nucleic acid sequence of European seabass (Dicentrarchus labrax) tac3, |
| | BK008116 |
| 67 | Amino acid sequence of European seabass (Dicentrarchus labrax) tac3 |
| 68 | Amino acid sequence of European seabass (Dicentrarchus labrax) tac3, first |
| | fragment (NKF) |
| 69 | Amino acid sequence of European seabass (Dicentrarchus labrax) tac3, |
| | second fragment (NKB) |
| 70 | Nucleic acid sequence of Tilapia Oreochromis niloticus |
| 71 | Amino acid sequence of Tilapia Oreochromis niloticus |
| 72 | Amino acid sequence of Tilapia Oreochromis niloticus, first fragment (NKF) |
| 73 | Amino acid sequence of Tilapia Oreochromis niloticus, second fragment |
| | (NKB) |
| 74 | Nucleic acid sequence of medaka (Oryzias latipes) tac3, BK008114 |
| 75 | Amino acid sequence of medaka (Oryzias latipes) tac3 |
| 76 | Amino acid sequence of medaka (Oryzias latipes) tac3, first fragment (NKF) |
| 77 | Amino acid sequence of medaka (Oryzias latipes) tac3, second fragment |
| | (NKB) |
| 78 | Nucleic acid sequence of Antarctic toothfish (Dissostichus mawsoni) tac3, |
| | BK008104 |
| 79 | Amino acid sequence of Antarctic toothfish (Dissostichus mawsoni) tac3 |
| 80 | Nucleic acid sequence of grass rockfish (Sebastes rastrelliger) tac3, |
| | BK008105 |
| 81 | Amino acid sequence of grass rockfish (Sebastes rastrelliger) tac3 |
| 82 | Nucleic acid sequence of Atlantic cod (Gadus morhua) tac3, BK008107 |
| 83 | Amino acid sequence of Atlantic cod (Gadus morhua) tac3 |
| 84 | Nucleic acid sequence of Arctic cod (Boreogadus saida) tac3, BK008109 |
| 85 | Amino acid sequence of Arctic cod (Boreogadus saida) tac3 |
| 86 | Nucleic acid sequence of western clawed frog (Xenopus tropicalis) tac3, |
| | BK008110 |
| 87 | Amino acid sequence of western clawed frog (Xenopus tropicalis) tac3 |
| 88 | Nucleic acid sequence of rainbow smelt (Osmerus mordax) tac3, BK008111 |
| 89 | Amino acid sequence of rainbow smelt (Osmerus mordax) tac3 |
| 90 | Nucleic acid sequence of American alligator (Alligator mississippiensis) |
| | tac3, BK008115 |
| 91 | Amino acid sequence of American alligator (Alligator mississippiensis) tac3 |
| 92 | FXGLM |
| 93 | Amino acid sequence of Atlantic salmon (Salmo salar) tac3b, second |
| | fragment (NKBb) |
| 94 | Amino acid sequence of zebra fish full length tac3ra (zftac3ra) |
| 95 | Amino acid sequence of zebra fish full length tac3rb (zftac3rb) |
| 96 | Amino acid sequence of the first peptide fragment X¹-X²-X³-X⁴-X⁵-X⁶- |
| | Asp⁷-X⁸-Phe⁹-Val¹⁰-X¹¹-Leu¹²-Met¹³ |
| 97 | Amino acid sequence of the first peptide fragment with the DID motif |
| 98 | Amino acid sequence of the Dissostichus mawsoni (Antarctic toothfish) |
| | NKF peptide |
| 99 | Amino acid sequence of the Dissostichus mawsoni (Antarctic toothfish) |
| | NKBa peptide |
| 100 | Amino acid sequence of the Sebastes rastrelliger (grass rockfish) NKF |
| | peptide |
| 101 | Amino acid sequence of the Sebastes rastrelliger (grass rockfish) NKBa |
| | peptide |
| 102 | Amino acid sequence of the Gadus morhua (Atlantic cod) NKF peptide |
| 103 | Amino acid sequence of the Arctic cod (Boreogadus saida) NKF peptide |
| 104 | Amino acid sequence of the Arctic cod (Boreogadus saida) NKBa peptide |
| 105 | Amino acid sequence of the Osmerus mordax (rainbow smelt) NKF peptide |
| 106 | Amino acid sequence of the Osmerus mordax (rainbow smelt) NKBa peptide |

1. An isolated preprohormone or any active hormone peptide derived therefrom regulating reproduction in fish, wherein said preprohormone comprises a first and a second peptide fragments:
a. said first peptide fragment comprising the amino acid sequence of:
X¹-X²-X³-X⁴-X⁵-X⁶-Asp⁷-X⁸-Phe⁹-Val¹⁰-X¹¹-Leu¹²-Met¹³, as denoted by SEQ ID NO. 96, wherein
X¹, is Tyr or a hydrophobic, a polar or positively charged amino acid selected from Phe, Ser, Lys and Gln;
X², is Asn or Ser or a hydrophobic, a polar, an acidic or positively charged amino acid selected from His, Thr, Asp, Arg, Lys and Ile;
X³, is Asp or a hydrophobic or a polar amino acid selected from Phe and Arg;
X⁴, is Ile or Leu or a polar or a hydrophobic amino acid selected from Asp, Tyr and Val;
X⁵, is Asp or an hydrophobic amino acid Met;
X⁶, is Tyr or acidic amino acid selected from Asp or His;
X⁸, is Ser or a polar or hydrophobic amino acid selected from Thr, Phe and Val;
X¹¹, is Gly or a polar amino acid, Ser;
X¹³, is Met or a hydrophobic amino acid Leu, and
b. said second peptide fragment comprising the amino acid sequence of:
Glu¹-Met²-His³-Asp⁴-Ile⁵-Phe⁶-Val⁷-Gly⁸-Leu⁹-Met¹⁰ as denoted by SEQ ID NO. 40 and variants thereof, said variants comprise a substitution in at least one position selected from a group consisting of:
Glu¹ is any one of Asn, Asp and Tyr; His³ is any one of Asn and Asp; Asp⁴ is Gln; Ile⁵ is Val; Phe⁶ is Leu; Val⁷ is Ile; Gly⁸ is Ala; and Met¹⁰ is Leu;
wherein said active hormone peptide comprises at least one of said first and second peptide fragments.

2. The preprohormone according to claim 1, wherein said first peptide comprises an amino acid sequence as denoted by any one of SEQ ID NO. 42, 43, 53, 57, 61, 65, 68, 72, 76, 98, 100, 102, 103 and 105, or any analogs or derivatives thereof, said second peptide fragment comprises the amino acid sequence as denoted by any one of SEQ ID NO. 40, 41, 54, 58, 62, 69, 73, 77, 93, 99, 101, 104 and 106, or any analogs or derivatives thereof, wherein said preprohormone is designated NKBa (Neurokinin Ba) and comprises an amino acid sequence as denoted by any one of SEQ ID NO: 49, 52, 55, 56, 60, 67, 71, 75, 79, 81, 83, 85, 87, 89 and 91 or any analogues and derivatives thereof, and wherein said preprohormone is designated NKBb (Neurokinin Bb) and comprises an amino acid sequence as denoted by any one of SEQ ID NO: 50 and 64 and any analogues and derivatives thereof.

3-7. (canceled)

8. An isolated peptide comprising at least one of:
a. a first peptide fragment, wherein said first fragment is a trideca peptide of the amino acid sequence of:
X¹-X²-X³-X⁴-X⁵-X⁶-Asp⁷-X⁸-Phe⁹-Val¹⁰-X¹¹-Leu¹²-Met¹³, as denoted by SEQ ID NO. 96, wherein
X¹, is Tyr or a hydrophobic, a polar or positively charged amino acid selected from Phe, Ser, Lys and Gln;
X², is Asn or Ser or a hydrophobic, a polar, an acidic or positively charged amino acid selected from His, Thr, Asp, Arg, Lys and Ile;
X³, is Asp or a hydrophobic or a polar amino acid selected from Phe and Arg;
X⁴, is Ile or Leu or a polar or a hydrophobic amino acid selected from Asp, Tyr and Val;
X⁵, is Asp or an hydrophobic amino acid Met;
X⁶, is Tyr or acidic amino acid selected from Asp or His;
X⁸, is Ser or a polar or hydrophobic amino acid selected from Thr, Phe and Val;
X¹¹, is Gly or a polar amino acid, Ser;
X¹³, is Met or a hydrophobic amino acid Leu; and
b. a second peptide fragment comprising the amino acid sequence of:
Glu¹-Met²-His³-Asp⁴-Ile⁵-Phe⁶-Val⁷-Gly⁸-Leu⁹-Met¹⁰ as denoted by SEQ ID NO. 40 and variants thereof, said variants comprise a substitution in at least one position selected from a group consisting of:
Glu¹ is any one of Asn, Asp and Tyr; His³ is any one of Asn and Asp; Asp⁴ is Gln; Ile⁵ is Val; Phe⁶ is Leu; Val⁷ is Ile; Gly⁸ is Ala; and Met¹⁰ is Leu;
wherein said peptide regulates reproduction in fish.

9. The peptide according to claim 8 comprising said first peptide fragment of the amino acid sequence of:
X¹-X²-X³-X⁴-X⁵-X⁶-Asp⁷-X⁸-Phe⁹-Val¹⁰-X¹¹-Leu¹²-Met¹³, as denoted by SEQ ID NO. 96, wherein:
X¹, is Tyr or a hydrophobic, a polar or positively charged amino acid selected from Phe, Ser, Lys and Gln;
X², is Asn or Ser or a hydrophobic, a polar, an acidic or positively charged amino acid selected from His, Thr, Asp, Arg, Lys and Ile;
X³, is Asp or a hydrophobic or a polar amino acid selected from Phe and Arg;
X⁴, is Ile or Leu or a polar or a hydrophobic amino acid selected from Asp, Tyr and Val;
X⁵, is Asp or an hydrophobic amino acid Met;
X⁶, is Tyr or acidic amino acid selected from Asp or His;
X⁸, is Ser or a polar or hydrophobic amino acid selected from Thr, Phe and Val;
X¹¹, is Gly or a polar amino acid, Ser;
X¹³, is Met or a hydrophobic amino acid Leu.

10. The peptide according to claim 9, wherein said peptide is a trideca peptide comprising an amino acid sequence as denoted by any one of SEQ ID NO. 42, 43, 53, 57, 61, 65, 68, 72, 76, 98, 100, 102, 103 and 105, or any analogs or derivatives thereof.

11. (canceled)

12. The peptide according to claim 10, wherein said trideca peptide is designated NKFa and consists of the amino acid sequence of Tyr-Asn-Asp-Ile-Asp-Tyr-Asp-Ser-Phe-Val-Gly-Leu-Met-NH₂, as denoted by SEQ ID NO:42, or any analogs and derivatives thereof and wherein said trideca peptide analog is Succ-Asp-Ser-Phe-N(Me)Val-Gly-Leu-Met-NH₂ as denoted by SEQ ID NO: 46.

13. (canceled)

14. The peptide according to claim 8, comprising said second peptide fragment, said peptide comprises the amino acid sequence of:
Glu¹-Met²-His³-Asp⁴-Ile⁵-Phe⁶-Val⁷-Gly⁸-Leu⁹-Met¹⁰ as denoted by SEQ ID NO. 40 and variants thereof, said variants comprise a substitution in at least one position selected from a group consisting of: Glu¹ is any one of Asn, Asp and Tyr; His³ is any one of Asn and Asp; Asp⁴ is Gln; Ile⁵ is Val; Phe⁶ is Leu; Val⁷ is Ile; Gly⁸ is Ala; and Met¹⁰ is Leu.

15. The peptide according to claim 14, comprising an amino acid sequence as denoted by any one of SEQ ID NO. 40, 41, 54, 58, 62, 69, 73, 77, 93, 99, 101, 104 and 106, or any analogues and derivatives thereof.

16. The peptide according to claim 15, wherein said peptide is designated NKBa and consists of an amino acid sequence of Glu-Met-His-Asp-Ile-Phe-Val-Gly-Leu-Met-NH₂, as denoted by SEQ ID NO:40, and any analogues and derivatives thereof, said peptide analog is Succ-Asp-Ile-Phe-N(Me)Val-Gly-Leu-Met-NH₂ denoted by SEQ ID NO:44, and wherein said peptide is designated NKBb and consists of the amino acid sequence of Ser-Thr-Gly-Ile-Asn-Arg-Glu-Ala-His-Leu-Pro-Phe-Arg-Pro-Asn-Met-Asn-Asp-Ile-Phe-Val-Gly-Leu-Leu-NH₂, as denoted by SEQ ID NO: 41, and any analogues and derivatives thereof, said peptide analog is Succ-Asp-Phe-N(Me)Val-Gly-Leu-Met-NH₂ denoted by SEQ ID NO:45.

17-22. (canceled)

23. A composition comprising at least one of an isolated active hormone peptide regulating reproduction in fish as defined in claim 8, any analogues thereof, any preprohormone thereof, any nucleic acid sequence encoding said preprohormone or any combinations thereof and a pharmaceutically acceptable carrier, excipient or diluent.

24. The composition according to claim 23, wherein said composition comprises an effective amount of at least one of an isolated active hormone peptide as denoted by any one of SEQ ID NO. 42, 40, 41, 43, 53, 54, 57, 58, 61, 62, 65, 68, 69, 72, 73, 76, 77, 93, 98, 99, 100, 101, 102, 103, 104, 105 and 106, any analogues thereof, as denoted by any one of SEQ ID NO. 44, 46 and 45, any preprohormone thereof as denoted by any one of SEQ ID NO. 49, 50 52, 56, 60, 64, 67, 71, 75, 79, 81, 83, 85, 89, 87, and 91 or any nucleic acid sequence encoding said preprohormone, as denoted by any one of SEQ ID NO. 4, 5, 51, 55, 59, 63, 66, 70, 74, 78, 80, 82, 84, 86, 88 and 90 and any combinations thereof.

25. The composition according to claim 24, wherein said composition comprises an effective amount of at least one trideca active hormone peptide, said peptide is designated NKFa and consists of the amino acid sequence of Tyr-Asn-Asp-Ile-Asp-Tyr-Asp-Ser-Phe-Val-Gly-Leu-Met-NH₂, as denoted by SEQ ID NO: 42, or any analogs and derivatives thereof, and wherein said trideca peptide analog is Succ-Asp-Ser-Phe-N(Me)Val-Gly-Leu-Met-NH₂ as denoted by SEQ ID NO:46.

26. (canceled)

27. The composition according to claim 24, wherein said composition comprises an effective amount of at least one active hormone peptide, said peptide is designated NKBa and consists of the amino acid sequence of Glu-Met-His-Asp-Ile-Phe-Val-Gly-Leu-Met NH₂, as denoted by SEQ ID NO:40, and any analogues and derivatives thereof, said peptide analog is Succ-Asp-Ile-Phe-N(Me)Val-Gly-Leu-Met-NH₂, as denoted by SEQ ID NO: 44 and wherein said composition comprises an effective amount of at least one active hormone peptide, said peptide is designated NKBb and consists of the amino acid sequence of Ser-Thr-Gly-Ile-Asn-Arg-Glu-Ala-His-Leu-Pro-Phe-Arg-Pro-Asn-Met-Asn-Asp-Ile-Phe-Val-Gly-Leu-Leu-NH₂, denoted by SEQ ID NO:41, and analogues and derivatives thereof, said peptide analog is Succ-Asp-Phe-N(Me)Val-Gly-Leu-Met-NH₂ denoted by SEQ ID NO:45.

28-30. (canceled)

31. The composition according to claim 23, wherein said isolated active hormone peptide is presented in an amount effective for regulating reproduction in fish, and wherein said regulation of reproduction in fish comprises at least one of: advancing the onset of puberty, regulating the timing and amount of ovulation and spawning, synchronization or stimulation of reproduction, enhancing the development of gammets, enhancing vitellogenesis, induction of Gonadotropin-releasing hormone (GnRH), induction of Kisspeptine, increase in the levels of hypothalamic neurohormones, increasing the level of Luteinizing-hormone (LH) or Follicle-stimulating hormone (FSH) and induction of oocyte maturation.

32. (canceled)

33. A method for regulating reproduction in fish comprising the step of administering to said fish an effective amount of at least one of an isolated active hormone peptide, any analogues thereof, any preprohormone thereof or any nucleic acid sequence encoding said preprohormone, any combinations thereof and any composition comprising the same, wherein said preprohormone comprises a first and a second peptide fragments:
a. said first peptide fragment comprising the amino acid sequence of:
X-X²-X³-X⁴-X⁵-X⁶-Asp⁷-X⁸-Phe⁹-Val¹⁰-X¹¹-Leu¹²-Met¹³, as denoted by SEQ ID NO. 96, wherein
X¹, is Tyr or a hydrophobic, a polar or positively charged amino acid selected from Phe, Ser, Lys and Gln;
X², is Asn or Ser or a hydrophobic, a polar, an acidic or positively charged amino acid selected from His, Thr, Asp, Arg, Lys and Ile;
X³, is Asp or a hydrophobic or a polar amino acid selected from Phe and Arg;
X⁴, is Ile or Leu or a polar or a hydrophobic amino acid selected from Asp, Tyr and Val;
X⁵, is Asp or an hydrophobic amino acid Met;
X⁶, is Tyr or acidic amino acid selected from Asp or His;
X⁸, is Ser or a polar or hydrophobic amino acid selected from Thr, Phe and Val;
X¹¹, is Gly or a polar amino acid, Ser;
X¹³, is Met or a hydrophobic amino acid Leu, and
b. said second peptide fragment comprising the amino acid sequence of:
Glu¹-Met²-His³-Asp⁴-Ile⁵-Phe⁶-Val⁷-Gly⁸-Leu⁹-Met¹⁰ as denoted by SEQ ID NO. 40 and variants thereof, said variants comprise a substitution in at least one position selected from a group consisting of:
Glu¹ is any one of Asn, Asp and Tyr; His³ is any one of Asn and Asp; Asp⁴ is Gln; Ile⁵ is Val; Phe⁶ is Leu; Val⁷ is Ile; Gly⁸ is Ala; and Met¹⁰ is Leu;
wherein said active hormone peptide comprises at least one of said first and second peptide fragments.

34. The method according to claim 33, comprising the step of administering to said fish an effective amount of at least one of an isolated active hormone peptide as denoted by any one of SEQ ID NO. 42, 40, 41, 43, 53, 54, 57, 58, 61, 62, 65, 68, 69, 72, 73, 76, 77, 93, 98, 99, 100, 101, 102, 103, 104, 105 and 106, any analogues thereof, as denoted by any one of SEQ ID NO. 44, 46 and 45, any combinations thereof, any preprohormone thereof as denoted by any one of SEQ ID NO. 49, 50 52, 56, 60, 64, 67, 71, 75, 79, 81, 83, 85, 89, 87, and 91 or any nucleic acid sequence encoding said preprohormone, as denoted by any one of SEQ ID NO. 4, 5, 51, 55, 59, 63, 66, 70, 74, 78, 80, 82, 84, 86, 88 and 90.

35. The method according to claim 34, comprising the step of administering to said fish an effective amount of at least one trideca active hormone peptide, said peptide is designated NKFa and consists of the amino acid sequence of Tyr-Asn-Asp-Ile-Asp-Tyr-Asp-Ser-Phe-Val-Gly-Leu-Met-NH₂, as denoted by SEQ ID NO: 42, or any analogs and derivatives thereof and said trideca peptide analog is Succ-Asp-Ser-Phe-N(Me)Val-Gly-Leu-Met-NH₂ as denoted by SEQ ID NO:46.

36. (canceled)

37. The method according to claim 34, comprising the step of administering to said fish an effective amount of at least one active hormone peptide, said peptide is designated NKBa and consists of the amino acid sequence of Glu-Met-His-Asp-Ile-Phe-Val-Gly-Leu-Met NH₂, as denoted by SEQ ID NO:40, and any analogues and derivatives thereof and said peptide analog is Succ-Asp-Ile-Phe-N(Me)Val-Gly-Leu-Met-NH₂, as denoted by SEQ ID NO: 44.

38. (canceled)

39. The method according to claim 34, comprising the step of administering to said fish an effective amount of at least one active hormone peptide, said peptide is designated NKBb and consists of the amino acid sequence of Ser-Thr-Gly-Ile-Asn-Arg-Glu-Ala-His-Leu-Pro-Phe-Arg-Pro-Asn-Met-Asn-Asp-Ile-Phe-Val-Gly-Leu-Leu-NH₂, denoted by SEQ ID NO:41, and analogues and derivatives thereof and said peptide analog is Succ-Asp-Phe-N(Me)Val-Gly-Leu-Met-NH₂, as denoted by SEQ ID NO:45.

40. (canceled)

41. The method according to claim 33, wherein said regulation of reproduction in fish comprises at least one of: advancing the onset of puberty, regulating the timing and amount of ovulation and spawning, synchronization or stimulation of reproduction, enhancing the development of gammets, enhancing vitellogenesis, induction of Gonadotropin-releasing hormone (GnRH), induction of Kisspeptine, increase in the levels of hypothalamic neurohormones, increasing the level of Luteinizing-hormone (LH) or Follicle-stimulating hormone (FSH) and induction of oocyte maturation.

42-44. (canceled)


SEQUENCE LISTING (106 sequences)

SEQ ID NO: 1 - protein, 116 aa, Mus musculus
  MISC_FEATURE: mouse Tac2, NP_033338.2
MRSAMLFAAVLALSLAWTFGAVCEEPQGQGGRLSKDSDLYQLPPSLLRRLYDSRPVSLEGLLKVLSKASVGPKETSLPQKRDMHDFFVGLMGKRNSQPDTPTDVVEENTPSFGILK

SEQ ID NO: 2 - protein, 143 aa, Monodelphis domestica
  MISC_FEATURE: XP_001365310
MRTLLLMAILVISIGRSCHAVCEESKEQGAFGGGHSKVLDLYQLPPSLLRRLYNSRSISLDGLLRLLSKTSVDSKETMDYQKRDMHDFFVGLMGKRNIQADETLEELSKSLDLGTGADFFLLIEEEGKTPEETIPAGKGALGN

SEQ ID NO: 3 - protein, 14 aa, Artificial Sequence
  ornAna1, Contig391396403-6445
GDMHDFFVGLMGKR

SEQ ID NO: 4 - DNA, 502 bp, Danio rerio
  misc_feature: zebra fish full length tac3a (zftac3a), JN392856
ccctgtctctgtgtcttgtctgatagatagtccatcacaaaaggatttaactctccaagcagaaactggaactgagctcttttctacagatctacatcctcagctcagagtaatctgtaaagatgtaccgtggacttgtgttactgttcttggttttggtgctggaaactcgatggagtgagtcgagctgtcagcagtcagagtctcaaagatcagtttcaagcgagagtccaagttttcggatgtcgactcataacttgctgaagaggtataatgacatagattatgacagtttcgtcggattaatggggcgcagaaacgccgaaacagatgatataccaccccaacgtaaaagggaaatgcacgatatctttgttggactcatgggtcgacgaagcgctgaacctgaatccggacgtcaatggaggaaagagtacccagaaccaagcggaggaatcttcttcaacaaatgcaaactgaggtttcgtcgtgggttataggc

SEQ ID NO: 5 - DNA, 405 bp, Danio rerio
  misc_feature: zebra fish full length tac3b (zftac3b), JN392857
ctgaacagcatcctgttaacaccagctcatacgagtacttggataaggtgtgcgaggatgtcctgcggctggctgctcgcgctgctcgtccacgtgctgctgctgctcgcgtgcccgagactctcgcggagcgccctcgactactccttcactgacaacagcgacgcccagccggagcgctacgacaaacgatatgatgatattgattacgacagtttcgtcggcctgatgggcaggaggagcacaggaataaatcgtgaggcacatttgccatttagaccgaatatgaatgacatctttgtcggactgttaggacggagaaacactttgtcgtctatgagaaaagaaaggagagggaacattttcttcaaggatggaagactgaggttttgctgtggtgtatga

SEQ ID NO: 6 - DNA, 1269 bp, Danio rerio
  misc_feature: zebra fish full length tac3ra (zftac3ra), JF317292
tatatctaaatatttctggacatttctggcatggcacagtcacagaacggatctaacctaacggggaactttacgaaccagttcgtgcagccgccgtggcgcgtggcgctgtggtcggtggcgtacagctccatcctggcgatcgcggtgttcgggaatctgatcgtcatgtggatcattctggctcataagcggatgcgaaccgtcaccaactactttctgctcaacctggcgttttcggacgcctccatggccgccttcaacactttgatcaatttcgtttacgccacacacggagattggtatttcggagaagcctactgcaaatttcacaactttttccccgtcacctccgtgtttgccagcatttactccatgagcgcaatcgcagtcgacaggtacatggccatcatccatcctctgaaaccacgactctcggcgacggccaccaaagtgatcattgtgtgtatctgggtgctcgctgtggttttggccttcccgctgtgtttcttttcaaccatcaaaaaactgcccaaacgaactctctgctatgttgcctggccgagaccttcagaagaccctttcatgtatcatatcattgtggcgatgctggtgtatgttctgccgctggtggtcatgggtatcaactacactattgtcggattgaccctttggggaggagagattcctggtgactcctcagacaactatcagggccagctcagggccaagaggaaggtggtgaaaatgatgatcattgtagtggtgacctttgccttctgctggctgccgtaccatgtgtatttcctggtgacgggattgaacaagcagctggctcgatggaagttcattcagcagatctatctgtccatcatgtggcttgccatgagctccaccatgtataaccccattatttactgctgcctaaacagccggtttcgcgctggcttcaaacgtgttttccgctggtgcccttttgtgcaagtctctgactatgacgagcttgagctgcgggctatgaggcataaagtagcgcggcagagcagcatgtacacaatgtcacgaatggagaccaccgtagtcaccgtgtgtgacccatcagagccaaacacccagccaggccggaagagcctgcttaaccaccaccaccaccacaacggctgctccaacccagccaagagcaaagaaataacatacatgcaaagcgacccgaaggaggaattctcctgagaaggacttttgatgtaagattcacactgaagcattaag

SEQ ID NO: 7 - DNA, 1318 bp, Danio rerio
  misc_feature: zebra fish full length tac3rb (zftac3rb), JF317293
tttaagaaggatttcacggttaaatctaccatggctggtcctcagagcggctcaaatgtgacgcgtaatttcacaaatcagttcgtgcagccgccgtggcgggtcgccgtctggtcggtcgcttacagctcggtgctcgcggtcgccgtgttcggaaacctcattgttatttggatcattttggcccataaacggatgcgcaccgtcaccaactattttttgctcaacctggcgttttccgacgcgtccatggccgccttcaacacgctcatcaacttcatttacgccacgcacggagagtggtacttcggagaggtttactgcaagttccacaacttcttccctgtgaccgccgtgtttgccagcatttactccatgacagcgattgcagtcgacaggtacatggccataatacatcctctgaagcctcgtctgtcagccacggctactaaagtggtgattgtctgtatttgggcactggcagtgattttggctttcccgctgtgtttctactccaccacgagaaccatgcctcgcagaaccatttgctacgtcgcctggccaagaccggctgaggattcattcatgtatcacatcatagtgacggtgctggtctacatgctgcccctagtggtgatgggcatcacctacactatagtcggggttacactttggggaggagagattcctggagactcgtcggacaattatgttggacagctacgtgctaagaggaaggtggtgaagatgatgatcgtggtggtggtgactttcgccctctgctggttgccgtatcacatctatttcatcgtaacaggcctgaacaaacgcctgaacaagtggaagtccatccagcaggtgtatctgtctgtgctgtggctggccatgagctccaccatgtacaaccccatcatttactgctgtctgaatggcagatttcgcgcgggcttcaagcgggccttcaggtggtgtcccttcattcaggtgtccagctatgacgaactggaactccgtcccacccggctccatccacgcaaccagagcagcatgtgcaccctgtcccgcgtcgacaccagcctccatggtgaggacccacgacgcagtcagcggaagagcaccaaatcccaatgtctggtggaggtcagagacgaaaacacaccagccacgaaactctgtcttaatagagatcaagcgttcgcaacagagcagctcagctgaagagtgcatgattatagaattaaagcatattctaaaaatgcatttaagtgtgcattgagactcaaagctgcagcgtgatgaggttacactgcctccaagt

SEQ ID NO: 8 - DNA, 21 bp, Artificial Sequence
  quantitative real time PCR, zf  ef1a-1237F
aagacaaccccaaggctctca

SEQ ID NO: 9 - DNA, 21 bp, Artificial Sequence
  quantitative real time PCR, zf  ef1a-1419R
cctttggaacggtgtgattga

SEQ ID NO: 10 - DNA, 19 bp, Artificial Sequence
  quantitative real time PCR, GnRH2-36F
gctgatgctgtgtctgagt

SEQ ID NO: 11 - DNA, 20 bp, Artificial Sequence
  quantitative real time PCR, GnRH2-196R
tgtcttgaggatgtttcttc

SEQ ID NO: 12 - DNA, 19 bp, Artificial Sequence
  quantitative real time PCR, GnRH3-47F
gtgtgttggaggtcagtct

SEQ ID NO: 13 - DNA, 20 bp, Artificial Sequence
  quantitative real time PCR, GnRH3-208R
tccacctcattcactatgtg

SEQ ID NO: 14 - DNA, 22 bp, Artificial Sequence
  quantitative real time PCR, kiss1-10F
acagacactcgtcccacagatg

SEQ ID NO: 15 - DNA, 21 bp, Artificial Sequence
  quantitative real time PCR, kiss1-210R
caatcgtgtgagcatgtcctg

SEQ ID NO: 16 - DNA, 19 bp, Artificial Sequence
  quantitative real time PCR, kiss2-137F
gcgttttctgtcaatggag

SEQ ID NO: 17 - DNA, 19 bp, Artificial Sequence
  quantitative real time PCR, kiss2-317R
cgcttcgtttctctttccg

SEQ ID NO: 18 - DNA, 18 bp, Artificial Sequence
  quantitative real time PCR, kiss1ra-856F
cctaacttcaaggccaac

SEQ ID NO: 19 - DNA, 18 bp, Artificial Sequence
  quantitative real time PCR, kiss1ra-1095R
cctctcagtgttgctttc

SEQ ID NO: 20 - DNA, 18 bp, Artificial Sequence
  quantitative real time PCR, kiss1rb-755F
agacgtcatcggagcgtg

SEQ ID NO: 21 - DNA, 23 bp, Artificial Sequence
  quantitative real time PCR, kiss1rb-1041R
cctccttttgaagatcagaggac

SEQ ID NO: 22 - DNA, 20 bp, Artificial Sequence
  quantitative real time PCR, zf  tac3a-F29
tggttttggtgctggaaacc

SEQ ID NO: 23 - DNA, 19 bp, Artificial Sequence
  quantitative real time PCR, zf  tac3a-R191
tctgtttcggcgtttctgc

SEQ ID NO: 24 - DNA, 22 bp, Artificial Sequence
  quantitative real time PCR, zf  tac3b-F86
ctccttcactgacaacagcgac

SEQ ID NO: 25 - DNA, 22 bp, Artificial Sequence
  quantitative real time PCR, zf  tac3b-R246
gtttctccgtcctaacagtccg

SEQ ID NO: 26 - DNA, 20 bp, Artificial Sequence
  quantitative real time PCR, zf  tac3ra F154
gctcataagcggatgcgaac

SEQ ID NO: 27 - DNA, 19 bp, Artificial Sequence
  quantitative real time PCR, zf  tac3ra R334
tggcaaacacggaggtgac

SEQ ID NO: 28 - DNA, 20 bp, Artificial Sequence
  quantitative real time PCR, zf  tac3rb F343
tccatgacagcgattgcagt

SEQ ID NO: 29 - DNA, 21 bp, Artificial Sequence
  quantitative real time PCR, zf  tac3rb R523
cgtagcaaatggttctgcgag

SEQ ID NO: 30 - DNA, 22 bp, Artificial Sequence
  in situ hybridization or cloning, zf  tac3a F-122
ccctgtctctgtgtcttgtctg

SEQ ID NO: 31 - DNA, 21 bp, Artificial Sequence
  in situ hybridizationor cloning, zf tac3a  R360
gcctataacccacgacgaaac

SEQ ID NO: 32 - DNA, 20 bp, Artificial Sequence
  in situ hybridizationor cloning, zf  tac3b F-17
ggataaggtgtgcgaggatg

SEQ ID NO: 33 - DNA, 24 bp, Artificial Sequence
  in situ hybridizationor cloning zf tac3b stop
tcatacaccacagcaaaacctcag

SEQ ID NO: 34 - DNA, 20 bp, Artificial Sequence
  cloning, zf  tac3Ra start
atggcacagtcacagaacgg

SEQ ID NO: 35 - DNA, 20 bp, Artificial Sequence
  cloning, zf  tac3Ra  stop
tcaggagaattcctccttcg

SEQ ID NO: 36 - DNA, 20 bp, Artificial Sequence
  cloning, zf  tac3Rb start
atggctggtcctcagagcgg

SEQ ID NO: 37 - DNA, 21 bp, Artificial Sequence
  cloning, zf  tac3Rb stop
tcagctgagctgctctgttgc

SEQ ID NO: 38 - DNA, 260 bp, Danio rerio
  misc_feature: zebra fish tac3a (position -122 to 380)
agatgtaccgtggacttgtgttactgttcttggttttggtgctggaaactcgatggagtgagtcgagctgtcagcagtcagagtctcaaagatcagtttcaagcgagagtccaagttttcggatgtcgactcataacttgctgaagaggtataatgacatagattatgacagtttcgtcggattaatggggcgcagaaacgccgaaacagatgatataccaccccaacgtaaaagggaaatgcacgatatctttgttgga

SEQ ID NO: 39 - DNA, 240 bp, Danio rerio
  misc_feature: zebra fish tac3a (position 122 to 360)
agatgtaccgtggacttgtgttactgttcttggttttggtgctggaaactcgatggagtgagtcgagctgtcagcagtcagagtctcaaagatcagtttcaagcgagagtccaagttttcggatgtcgactcataacttgctgaagaggtataatgacatagattatgacagtttcgtcggattaatggggcgcagaaacgccgaaacagatgatataccaccccaacgtaaaagggaaa

SEQ ID NO: 40 - protein, 10 aa, Danio rerio
  MISC_FEATURE: zebra fish Tac3a second fragment peptide (NKBa)
EMHDIFVGLM

SEQ ID NO: 41 - protein, 24 aa, Danio rerio
  MISC_FEATURE: zebra fish Tac3b second fragment peptide (NKBb)
STGINREAHLPFRPNMNDIFVGLL

SEQ ID NO: 42 - protein, 13 aa, Danio rerio
  MISC_FEATURE: zebra fish first fragment peptide Tac3af (NKFa)
YNDIDYDSFVGLM

SEQ ID NO: 43 - protein, 13 aa, Danio rerio
  MISC_FEATURE: zebra fish first fragment peptide Tac3bf (NKFb)
YDDIDYDSFVGLM

SEQ ID NO: 44 - protein, 8 aa, Artificial Sequence
  NKBa-analog
XDIFXGLM

SEQ ID NO: 45 - protein, 7 aa, Artificial Sequence
  NKBb-analog
XDFXGLM

SEQ ID NO: 46 - protein, 8 aa, Artificial Sequence
  NKF-analog
XDSFXGLM

SEQ ID NO: 47 - protein, 7 aa, Artificial Sequence
  Senktide (NKB agonist)
XDFXGLM

SEQ ID NO: 48 - protein, 10 aa, Homo sapiens
  MISC_FEATURE: mammalian NKB
DMHDFFVGLM

SEQ ID NO: 49 - protein, 125 aa, Danio rerio
  MISC_FEATURE: zebra fish full length tac3a
MYRGLVLLFLVLVLETRWSESSCQQSESQRSVSSESPSFRMSTHNLLKRYNDIDYDSFVGLMGRRNAETDDIPPQRKREMHDIFVGLMGRRSAEPESGRQWRKEYPEPSGGIFFNKCKLRFRRGL

SEQ ID NO: 50 - protein, 115 aa, Danio rerio
  MISC_FEATURE: zebra fish full length tac3b
MSCGWLLALLVHVLLLLACPRLSRSALDYSFTDNSDAQPERYDKRYDDIDYDSFVGLMGRRSTGINREAHLPFRPNMNDIFVGLLGRRNTLSSMRKERRGNIFFKDGRLRFCCGV

SEQ ID NO: 51 - DNA, 413 bp, Pimephales promelas
  misc_feature: fathead  minnow (Pimephales promelas) tac3, BK008100
accgtggactagtgttactgttcttggttttggtgctggaaacccgatggagtgaatcgagctgtcagcagtcagaatctcaaagatcagattcaaacgagattccaagtttgcggctgtcggctcataacttgctgaagaggtataatgacatagattatgacagtttcgtcggattaatgggtcgcagaaacgccgacacggatgatttcccaccccaacgtaaaagggaaatgcacgatatctttgttggactcatgggtagacgaagcgctgaccctgaaactgggcgtccgttgaggaaagactatccagaaacaagcggaggaggcatcttcttcaacaaatgcaaactgaggtttcgtcgtgggttataggcaaatggattatttcatatccagttaatcgtctgt

SEQ ID NO: 52 - protein, 124 aa, Pimephales promelas
  MISC_FEATURE: fathead minnow (Pimephales promelas) tac3
RGLVLLFLVLVLETRWSESSCQQSESQRSDSNEIPSLRLSAHNLLKRYNDIDYDSFVGLMGRRNADTDDFPPQRKREMHDIFVGLMGRRSADPETGRPLRKDYPETSGGGIFFNKCKLRFRRGL

SEQ ID NO: 53 - protein, 13 aa, Pimephales promelas
  MISC_FEATURE: fathead minnow (Pimephales promelas) tac3 first fragment
YNDIDYDSFVGLM

SEQ ID NO: 54 - protein, 10 aa, Pimephales promelas
  MISC_FEATURE: fathead minnow (Pimephales promelas) tac3 second fragment
EMHDIFVGLM

SEQ ID NO: 55 - DNA, 609 bp, Ictalurus punctatus
  misc_feature: channel catfish (Ictalurus punctatus) tac3, BK008101
gacttagctgatgcagcgatcatctatgcagatatctgctctgacaggatatcgaggtacagctttaaccgtgagctcctcttctaacgcatccaagtgactcggccgtgcgcctcacctgtgcctcagacgatttatcagctcagactgtgtgtaaaaatgtatcgaggattgctgttggtgttcctcatccttctggttgagactcggctcagtgagtcgagctgcgcggaacaagagacgcagagatcagcctcggacgagagttcaagtttccgactcgcgactcgtaacttgctgaagcggtatcacgacatagactacgacagttttgttggactgatgggccgcaggagcgccgatgctgctgctgctgctgaggaccagtctcagcgtaaaagagaaatgcatgacatctttgtgggacttatgggacggcgaaacagtgaagatgacactgaacgtccatggagaaaagactacccagagacacgtggcctgttctttaacaagtgcagactgaggtttcgtcgtgggttataggcaaaaatcctctggaccagctgttccgttctcacaggatgaactccttttcatgacacactttgc

SEQ ID NO: 56 - protein, 127 aa, Ictalurus punctatus
  MISC_FEATURE: channel catfish (Ictalurus punctatus) tac3
MYRGLLLVFLILLVETRLSESSCAEQETQRSASDESSSFRLATRNLLKRYHDIDYDSFVGLMGRRSADAAAAAEDQSQRKREMHDIFVGLMGRRNSEDDTERPWRKDYPETRGLFFNKCRLRFRRGL

SEQ ID NO: 57 - protein, 13 aa, Ictalurus punctatus
  MISC_FEATURE: channel catfish (Ictalurus punctatus) tac3 first fragment
YHDIDYDSFVGLM

SEQ ID NO: 58 - protein, 10 aa, Ictalurus punctatus
  MISC_FEATURE: channel catfish (Ictalurus punctatus) tac3, second fragment
EMHDIFVGLM

SEQ ID NO: 59 - DNA, 904 bp, Salmo salar
  misc_feature: Atlantic salmon (Salmo salar) tac3a, BK008102
gggggtcctcgtggatctcgtcgttgcactgcaggagctttccgtatcatcgtttcgctctcaccactaagcatctgcacccgggcgcgcgcacggtcttctccagctgtctctaacataatattccaattcgaatattaccacgtcggaaaacagcttgtctaggctacaggtggtgttaagaagatgaaaaacggattgttactggtgactctgttcctcgtcatgaaactccgctcaagccagtccagctgcgaggagcccgctgctggggcacacagatcaactgctgactcagaggatactccaggtcttgaaaagctcaagcggtccattctgaagagatataacgacctggattacgacagcttcgttgggctgatggggagaaggggcgctgatatttatgacctacctccctctccgcataaaagagagatggatgatgtctttgttgggcttatgggcagaagaaacttggaacaaggtaacatgcgtcctttgagaaaggaggcataccctgagacaagaagaggaggcttattcttcaataagtgcagactgaggtttcggcgtgggctgtagacaagacagtcatccagaacccatccctaacatccagccaaggcaaaaccccttttcagaagattggagttattattatacaacacagaaaaagacaactatttattttacaaaacaaaagtattttgcgaaaacatctatggaawttaaactgaccaaaatcaacctgctactgtaacatgtgccctgatgggttgtcatagtaactaattgctatataatgtattatctgaaagatatccgtaacagtcccatctgataagatctatttctttaccatatgtttgtgattcaaatgaaataaataataaaatgtggc

SEQ ID NO: 60 - protein, 132 aa, Salmo salar
  MISC_FEATURE: Atlantic salmon (Salmo salar) tac3a
MKNGLLLVTLFLVMKLRSSQSSCEEPAAGAHRSTADSEDTPGLEKLKRSILKRYNDLDYDSFVGLMGRRGADIYDLPPSPHKREMDDVFVGLMGRRNLEQGNMRPLRKEAYPETRRGGLFFNKCRLRFRRGL

SEQ ID NO: 61 - protein, 13 aa, Salmo salar
  MISC_FEATURE: Atlantic salmon (Salmo salar) tac3a, first fragment (NKF)
YNDLDYDSFVGLM

SEQ ID NO: 62 - protein, 10 aa, Salmo salar
  MISC_FEATURE: Atlantic salmon (Salmo salar) tac3a, second fragment (NKBa)
EMDDVFVGLM

SEQ ID NO: 63 - DNA, 979 bp, Salmo salar
  misc_feature: Atlantic salmon tac3b, BK008103
atcaggttctctctgtcatcgacataagattgaacaaatatcaataagtttgcagagaacactatcccacctggacagcagagagatgatgagacacggctgtgcgcaggccatattgactttcttggttatcctcatctcatgtcccatggagtcagactgtgaagaggactcctacaactcaatactgcaggacagcccatattacctcagcagtggtttaactaattttaagaggtacagggacattcacgacgacacctttgtgggtctgatgggcaggaggagtgcaggtgtaaatgatctaccgtcacgaagaagtaagatcagggacatggatgatgtctttgtgggcctcctgggtcgcaggagctccgggtcagccatccctcagccctggagggaggaggtatacccacagccaagcggaggcattctcatcaagaaagggagactgagatttgtacccggtgtatagggtcccgcctcgtccctttcatccaaccaaacaccgcacagcagactgcactacgccagactgtatacacgtcattcatcctragttackttatttwccactaagaaaccattgtcttgctgtgataccactatttttcctttttgtcaccactataaaaggtgaatgaatgaatatctgtaacacacatgtaaagtattacaaatgaatgtaatgttatgttagacatggtcgtagtacattaggtatgttataccatatgaagggtgtctttctttaatatgctggttaaagcacttttcattaaattgattaaaatcaatttaactctaactcttcttaaacatacatatacaccaaacataaataaacagaggcagttctgaacaccgttccctttgacaagaatgtaaacttcacagaaccaaaacgcaacacttacaagtgttggtcccatgtttcatgagctgaaataaaagaccccagaaatgtcaaaaaaaa

SEQ ID NO: 64 - protein, 130 aa, Salmo salar
  MISC_FEATURE: Atlantic salmon (Salmo salar) tac3b
MMRHGCAQAILTFLVILISCPMESDCEEDSYNSILQDSPYYLSSGLTNFKRYRDIHDDTFVGLMGRRSAGVNDLPSRRSKIRDMDDVFVGLLGRRSSGSAIPQPWREEVYPQPSGGILIKKGRLRFVPGV

SEQ ID NO: 65 - protein, 13 aa, Salmo salar
  MISC_FEATURE: Atlantic salmon (Salmo salar) tac3b, first fragment  (NKF)
YRDIHDDTFVGLM

SEQ ID NO: 66 - DNA, 741 bp, Dicentrarchus labrax
  misc_feature: European seabass (Dicentrarchus labrax) tac3, BK008116
gaatttgacttgacagcctttaataccagtaaactgttgaacagaccactggtttgtactgctggatcagatcaagattaaactgtcaacagagtgatggagaggacttcaaactgctgcaccgttgcttcactggtcgttctagtcatcctcgtcctttttcctctgaggtcttggtgtaaggaagacatctacaaagcaacgtcacaggctaagccagagtgctgcattagtgaagatgtagagaagagatctgatgacatcgactacgacacctttgtgagtctaatggggaggagaagtgctgctcaaccaaacagtcaaagaaacgcccagatgtcaaagaaaagatacatggatcaaatcctcgctgctctgttggggcggagaacgagaatggtctgtccatgcctcgaggagtattcactgaaaattagaggctgactcatcaactacaggaggccgaggtttcaacactttctgtggaggagacatcgcacctgcacaacctcaacacaggctgtaaacctttcagtccctcacagcagtgaccaccaatcctcacagctattgtaccaactgaggacaatgtatcaaagctatttaaccacagagaggtggaaaagccatattaaccatgcatgaatgcctactacggtctctggaaaatgttctgacttgaatgcaaacaaatgtaaacaaatgtacaataaaatatgcatttatttatagtgaaaaaaa

SEQ ID NO: 67 - protein, 115 aa, Dicentrarchus labrax
  MISC_FEATURE: European seabass (Dicentrarchus labrax) tac3
MERTSNCCTVASLVVLVILVLFPLRSWCKEDIYKATSQAKPECCISEDVEKRSDDIDYDTFVSLMGRRSAAQPNSQRNAQMSKKRYMDQILAALLGRRTRMVCPCLEEYSLKIRG

SEQ ID NO: 68 - protein, 13 aa, Dicentrarchus labrax
  MISC_FEATURE: European seabass (Dicentrarchus labrax) tac3, first fragment
SDDIDYDTFVSLM

SEQ ID NO: 69 - protein, 10 aa, Dicentrarchus labrax
  MISC_FEATURE: European seabass (Dicentrarchus labrax) tac3, second fragment
YMDQILAALL

SEQ ID NO: 70 - DNA, 352 bp, Oreochromis niloticus
  misc_feature: Tilapia Oreochromis niloticus
atgagaagaggtttgctactggtgacttttttcctcgtcatggaatttcgctacagccggtgcagatgcgacgagcccggatcgcgcagatcaacttcagaccagaccataggcttagagaactttaagcggaacatcctgaagagatataatgacttggattatgacagctttgtgggtctgatggggagaagaagtgcagaggctgacgctgaacaaccaccccaaaaaagggagatggatgacatttttattggacttatgggacggagaaacgcagagactgataatggtccctggaggagagagtatccagagaggaggggcatttttctcaacaagtgcaggctga

SEQ ID NO: 71 - protein, 117 aa, Oreochromis niloticus
  MISC_FEATURE: Tilapia Oreochromis niloticus
MRRGLLLVTFFLVMEFRYSRCRCDEPGSRRSTSDQTIGLENFKRNILKRYNDLDYDSFVGLMGRRSAEADAEQPPQKREMDDIFIGLMGRRNAETDNGPWRREYPERRGIFLNKCRL

SEQ ID NO: 72 - protein, 13 aa, Oreochromis niloticus
  MISC_FEATURE: Tilapia Oreochromis niloticus, first fragment
YNDLDYDSFVGLM

SEQ ID NO: 73 - protein, 10 aa, Oreochromis niloticus
  MISC_FEATURE: Tilapia Oreochromis niloticus, second fragment
EMDDIFIGLM

SEQ ID NO: 74 - DNA, 554 bp, Oryzias latipes
  misc_feature: medaka (Oryzias latipes) tac3, BK008114
gtcattatggccggggaatgtagggaggatttggacgcatctgaagtagtatctcctctgcgccaggacgcacgcagctgactcttcctctacattagaaaacaggaaaaatgcgatcaggtttgctcctggtgactttgttcatctttgtgaaacttcgctacagccagtcgaggtgcgaggaaaccgcctcgcgcaggtcaacactggggcagcctataagtttggaagaattcaagcggaaccttctcaggaggtacactgacctggactatgacagctttgtaggactgatgggcaaaagaaacgcagaagaagaggctgtgcagtcacaaccaaaaagagacatggatgatatatttgtgggtcttatgggaagaagaagttcagagcctgagaatggcccctggaggagagactatccagacaggagaggcgtcttattcaacaagtccaggctgaggttccttcaaggactgtagactttctcacagaatccacgtattatttgctgacgttgacagaagatgcaccagaggaacataaataaccgt

SEQ ID NO: 75 - protein, 123 aa, Oryzias latipes
  MISC_FEATURE: medaka (Oryzias latipes) tac3
MRSGLLLVTLFIFVKLRYSQSRCEETASRRSTLGQPISLEEFKRNLLRRYTDLDYDSFVGLMGKRNAEEEAVQSQPKRDMDDIFVGLMGRRSSEPENGPWRRDYPDRRGVLFNKSRLRFLQGL

SEQ ID NO: 76 - protein, 13 aa, Oryzias latipes
  MISC_FEATURE: medaka (Oryzias latipes) tac3, first fragment
YTDLDYDSFVGLM

SEQ ID NO: 77 - protein, 10 aa, Oryzias latipes
  MISC_FEATURE: medaka (Oryzias latipes) tac3, second fragment
DMDDIFVGLM

SEQ ID NO: 78 - DNA, 594 bp, Dissostichus mawsoni
  misc_feature: Antarctic toothfish (Dissostichus mawsoni) tac3, BK008104
atgctggcctgatctcactgcaggatctatccttcttcacttctctgcaccagggcgcacgcagactcttttaccaaactatctgcttgacagaaaacaggaggtgctgagaaaatgaagggaggtgtgctgctggtgactctgttcctcatcatgaaacttcgatacagccattccagatgcgaggagcccggaacgcgcagatcagaaaagcccacagtcttggacaacctcaagcggaacattctgaagaggtatagcgatttggattatgacagctttgtaggtctgatgggcagaagagatgccgatgatagcgctgtacctgcaccacaaaaaagggaaatgaatgacatttttgtgggtcttatgggaaggagaaactcagaccctgatgatggtcccttgagaagagagtatccagagaggagaggcatttttctcaacaagtgcaggctgaggtttcttcaggggctgtaaaacttcattcagaagcaactctctcatctggcatcgatgacagaagatcacagaaaacacaacaaattaacaaaatgtctgatggaaatgaataactggcgtgattcacactgt

SEQ ID NO: 79 - protein, 121 aa, Dissostichus mawsoni
  MISC_FEATURE: Antarctic toothfish (Dissostichus mawsoni) tac3
MKGGVLLVTLFLIMKLRYSHSRCEEPGTRRSEKPTVLDNLKRNILKRYSDLDYDSFVGLMGRRDADDSAVPAPQKREMNDIFVGLMGRRNSDPDDGPLRREYPERRGIFLNKCRLRFLQGL

SEQ ID NO: 80 - DNA, 758 bp, Sebastes rastrelliger
  misc_feature: grass  rockfish (Sebastes rastrelliger) tac3, BK008105
cactgtaggatctatcagttcccactttcctctgcaccaggacgcacgcagacgactcttttaccaaactatctgcccgacagaaaacaggcggtgctgagaaaatgaggagaggtttgctgctggtgtttctgttcctcatcatgaaacttcgatacagccagtccagatgtgaggagcctggagcgcgcagatcaacttcagatcagaccacaggcttggacaacctcaagaggaacattctgaagaggtatagcgatttggattatgacagctttgtgggtctgatgggaagaagagatgcagatgcaaacgctgtgcagtcaccacaaaaaagggaaatgcatgacatttttgttggtcttatgggaaggagaaactcagagcctgataatggtccctggaggagagagaatacagagaggagaggcatttttctcaacaagtgcaggctgaggtttctacaggggctgtagacattcatataccgaagtaactctcatctggcatcaatgacggaagatcacagagaacacaaacaatgaactacatgtctgaaggaaacaactaaatggcgtgattcacactgtaaacctgccagctggcttatgaaagtgaccataaactacacagattgtatttgttttatctgaagaccaaacatgtttagtagcaacagccaacacgttctgtttctttatcatgttgttattcttttttcttcaaaacatcaaaataaacacaattttttatacacc

SEQ ID NO: 81 - protein, 123 aa, Sebastes rastrelliger
  MISC_FEATURE: grass rockfish (Sebastes rastrelliger) tac3
MRRGLLLVFLFLIMKLRYSQSRCEEPGARRSTSDQTTGLDNLKRNILKRYSDLDYDSFVGLMGRRDADANAVQSPQKREMHDIFVGLMGRRNSEPDNGPWRRENTERRGIFLNKCRLRFLQGL

SEQ ID NO: 82 - DNA, 598 bp, Gadus morhua
  misc_feature: Atlantic cod (Gadus morhua) tac3, BK008107
aactcaggatctactgctcacaactccacgcctggaccctttgccacccagttgtcagctcaacagaacgcaggcagagcaaagaagatgcgaataagtttggtgctggtcactctgtttcttgttatgaaactccgatccacccaatccagatgtgaggagcctgggtcgcgtagacccctctcagaccccagtttagtgaacaggcggaacattgtgaggaggtccagtgatttggattatgacagttttgtgggactgatgggcaggagaaacacagaggcggcaaatgaattaccgtcggcaaataaaaacaggggagatgcacgacatcttcgttggtcttatgggacggagaaactcagagacagatgatggtccatggaggaaaacggacccggagagaaggggagtcttcctgaataagtgtagactccggtttcggcgtggattttgatcctgcctttcagagtacatctcatctggctaagatgacacctcattaccgagcatacgaaatgcaaatgtctctgaacatggcacgggatattttacaatacaaccctccggaattgtttatggctgctgtttgtgtcac

SEQ ID NO: 83 - protein, 95 aa, Gadus morhua
  MISC_FEATURE: Atlantic cod (Gadus  morhua) tac3
MRISLVLVTLFLVMKLRSTQSRCEEPGSRRPLSDPSLVNRRNIVRRSSDLDYDSFVGLMGRRNTEAANELPSANKNRGDARHLRWSYGTEKLRDR

SEQ ID NO: 84 - DNA, 751 bp, Boreogadus saida
  misc_feature: Arctic cod (Boreogadus saida) tac3, BK008109
gggggagtcctctcctgatctcactgcaggatctactgctcacaactccacgccaggaccctttgccacccagttgtcagctcaacagaacgcaggcagagcaaagaagatgcgaataagtttggtgctggtcactctgtttcttgttatgaaactccgatccacccaatccagatgtgaggagcctggatcgcgtagacccctctcagaccccagtttagtgaacaggcggaacattgtgaggaggttcagtgatttggattatgacagttttgtgggactgatgggcaggagaaacacagaggcggcaaatgaattaccgtcggcaaatgaattaccgtcggcaaataaaagggaaatgcacgacatcttcgttggtcttatgggacggagaaactcagagacagatgatggtccatggaggaaaacggacccggagagaaggggagtcttcctgaataagtgtagactccggtttcggcgtggattttgatcctgcctttcagagtacatctcacctggctaagatgacacctcattaccgagcatacgaaatgcaaacgtctctgaacatggcacgggatattttacaatacaaccctccggattgtttatggctgctgttgtgttacatgtttgtagcctatatgaatgatctgaacaacaagcgttgtttagaattgaaacatgaatcgaattaattaatttatcttattccatgcaattaaaataaagcattttgtaaataatg

SEQ ID NO: 85 - protein, 127 aa, Boreogadus saida
  MISC_FEATURE: Arctic cod (Boreogadus saida) tac3
MRISLVLVTLFLVMKLRSTQSRCEEPGSRRPLSDPSLVNRRNIVRRFSDLDYDSFVGLMGRRNTEAANELPSANELPSANKREMHDIFVGLMGRRNSETDDGPWRKTDPERRGVFLNKCRLRFRRGF

SEQ ID NO: 86 - DNA, 890 bp, Xenopus tropicalis
  misc_feature: western clawed frog (Xenopus tropicalis) tac3, BK008110
ggggaaggcagctcccagctatccagcagcagcagcacagcggagcggagcagcactaccatatacagatcctgtggctgcaccccagagccagtgagaagatgaagaatttggcagttggtctagctgttcttattttggtggcagtgagaacatgccgtggaggctgtggggactcacaaggggagcagggatcaactacacagcttaagagaagttcagatatttacaagcttccagcttccctactgaagagattctacgatgatgattcatttgttgggcttatgggcaaaagaaattctgatttcaaggaattcccttctctgcctttaaaacgagaaatgaatgatttttttgttggtttaatgggaaaaaggaatttacaaacagggaatcccactgaggaagaaaatgaagcccgaccagacaccagatactcaacaaaatgtagaatgaagtttaggatgtagtgatcatcttctcaaccattcttcagacttcaaataccaaccaagttgaatttcttcttttgtaccaaggatacaaaacccacctgtattgcaatatctcacaagatcttcacaatataaaatgtccagccatacccgcattgatcattacaacatgatggattaaacaggaatgaaaaatggaaccataccccacagattctctgacattcacatctatctaagatattttatcagatttccagtcattttcttatatctagcttaatgatctagctcaatctgatctgcacttattatatcaggtgcttactgaggggtaattaggataaaggcacaacaatattcattggcattaaaacccacagtaaccaattaggcgtttgaacttgttcattgattactctttttattcaa

SEQ ID NO: 87 - protein, 123 aa, Xenopus tropicalis
  MISC_FEATURE: western clawed frog (Xenopus tropicalis) tac3
MKNLAVGLAVLILVAVRTCRGGCGDSQGEQGSTTQLKRSSDIYKLPASLLKRFYDDDSFVGLMGKRNSDFKEFPSLPLKREMNDFFVGLMGKRNLQTGNPTEEENEARPDTRYSTKCRMKFRM

SEQ ID NO: 88 - DNA, 983 bp, Osmerus mordax
  misc_feature: rainbow smelt (Osmerus mordax) tac3, BK008111
tgggggtccgcgcggatttcacagcaggatatatactgtccgttcatccctactcaccagtgcgcgcgcggtatccacaaccaccaacagacttggctactcataaaccaggtggtccaaagaagatgagaagaggattactattggtggcactgttcctcgtcatgaaactccgattgtgccagtccggatgcgaggagccagggtcgcgcaggtcatcgtcaggtgaaatcccaggtttaggtgagatgaagcgcaatcttctgaagaggtacagcgacgtggactatgacagttttgtgggtctgatgggtagacgaaatgcggatataaatggtgtacaatcccaacaaaaaagagaaatgcatgacatatttgttggactcatgggacggaggaattcagagcctggtaacgctggtccctggaggaaagctaacccagagacaaaaggcatattttacaacaagtgcagacttaggtttcgtcgcggcttgtagacgttcttcacactacagatgatgaatcttaagtttcattatttattttaaccttttacaaaatgaattaactaaactgacagagacatgcacaagttaattaaagcacctaggctttacagcataaagtttaaagtttgtataagacgttttatctgataaaagcaaaagctaaatgagaactatttctttaccatagtgttcctttcctccaagataaagtaaacctcatttttgtaaataaaaatggaaatcaacaattctgtttattactgctgatgtgaatccgttttggatgtatttcttaatttttccatccaggtcaatgtaaaatgtttattgttctgtgaattattcagctctgctcgtcttaggagggtgctatcttgagattggatggaaacaagttacccacctcaacatttgagatgcatacaccttaaattgttctgaatgctgaaatgcaccagacattactacagg

SEQ ID NO: 89 - protein, 124 aa, Osmerus mordax
  MISC_FEATURE: rainbow smelt (Osmerus mordax) tac3
MRRGLLLVALFLVMKLRLCQSGCEEPGSRRSSSGEIPGLGEMKRNLLKRYSDVDYDSFVGLMGRRNADINGVQSQQKREMHDIFVGLMGRRNSEPGNAGPWRKANPETKGIFYNKCRLRFRRGL

SEQ ID NO: 90 - DNA, 618 bp, Alligator mississippiensis
  misc_feature: American alligator (Alligator mississippiensis) tac3, BK008115
gcacgaggagcccggcagctgcagcggtgtcccgcacgggcgcgtgtgtaagggctccaacccgcccaggacagacatgaggggccaccttgtgctcacggtgctgctgtccctagcggcctggcggctgtgccaggctgagtgccgggaccccacggctggcaggacccagatccagaggagctccgacctgttcaagctgcccccctccctgctccggcggctgtaccagggggtctcctacgaggcacttttgcagctggctgacaaagccccagtgggtcttcaggctcttgcccccccccagaaacgcgacatgcacgacttcttcgtggggctcatggggaagaggacggttgtgcctgggagccctgtggacgagagccaggagccctttgccaccttcggagacccctgggactcgccgagcgcagagtgaggtcccgcgtgggcccagccgtgccctcgttccctccccctgcccagcatcatgccatggaggagccctgctcacgactgcctcatgccccttgtgcccccagtgctcactgttgtcacgtgcccttgacctctgacccctgaccccggaccggtcaagaataaagtttttactaacga

SEQ ID NO: 91 - protein, 120 aa, Alligator mississippiensis
  MISC_FEATURE: American alligator (Alligator mississippiensis) tac3
MRGHLVLTVLLSLAAWRLCQAECRDPTAGRTQIQRSSDLFKLPPSLLRRLYQGVSYEALLQLADKAPVGLQALAPPQKRDMHDFFVGLMGKRTVVPGSPVDESQEPFATFGDPWDSPSAE

SEQ ID NO: 92 - protein, 5 aa, Artificial Sequence
  FXGLM motif
FXGLM

SEQ ID NO: 93 - protein, 10 aa, Salmo salar
  MISC_FEATURE: Atlantic salmon (Salmo salar) tac3b, second fragment (NKBb)
DMDDVFVGLL

SEQ ID NO: 94 - protein, 399 aa, Danio rerio
  MISC_FEATURE: zebra fish full length tac3ra (zftac3ra)
MAQSQNGSNLTGNFTNQFVQPPWRVALWSVAYSSILAIAVFGNLIVMWIILAHKRMRTVTNYFLLNLAFSDASMAAFNTLINFVYATHGDWYFGEAYCKFHNFFPVTSVFASIYSMSAIAVDRYMAIIHPLKPRLSATATKVIIVCIWVLAVVLAFPLCFFSTIKKLPKRTLCYVAWPRPSEDPFMYHIIVAMLVYVLPLVVMGINYTIVGLTLWGGEIPGDSSDNYQGQLRAKRKVVKMMIIVVVTFAFCWLPYHVYFLVTGLNKQLARWKFIQQIYLSIMWLAMSSTMYNPIIYCCLNSRFRAGFKRVFRWCPFVQVSDYDELELRAMRHKVARQSSMYTMSRMETTVVTVCDPSEPNTQPGRKSLLNHHHHHNGCSNPAKSKEITYMQSDPKEEFS

SEQ ID NO: 95 - protein, 395 aa, Danio rerio
  MISC_FEATURE: zebra fish full length tac3rb (zftac3rb)
MAGPQSGSNVTRNFTNQFVQPPWRVAVWSVAYSSVLAVAVFGNLIVIWIILAHKRMRTVTNYFLLNLAFSDASMAAFNTLINFIYATHGEWYFGEVYCKFHNFFPVTAVFASIYSMTAIAVDRYMAIIHPLKPRLSATATKVVIVCIWALAVILAFPLCFYSTTRTMPRRTICYVAWPRPAEDSFMYHIIVTVLVYMLPLVVMGITYTIVGVTLWGGEIPGDSSDNYVGQLRAKRKVVKMMIVVVVTFALCWLPYHIYFIVTGLNKRLNKWKSIQQVYLSVLWLAMSSTMYNPIIYCCLNGRFRAGFKRAFRWCPFIQVSSYDELELRPTRLHPRNQSSMCTLSRVDTSLHGEDPRRSQRKSTKSQCLVEVRDENTPATKLCLNRDQAFATEQLS

SEQ ID NO: 96 - protein, 13 aa, Artificial Sequence
  Amino acid sequence of the first peptide fragment
XXXXXXDXFVXLM

SEQ ID NO: 97 - protein, 13 aa, Artificial Sequence
  Amino acid sequence of the first peptide fragment with the DID motif
XDIDXXDXFVXLM

SEQ ID NO: 98 - protein, 13 aa, Dissostichus mawsoni
  MISC_FEATURE: Amino acid sequence of the Dissostichus mawsoni (Antarctic toothfish) NKF peptide
YSDLDYDSFVGLM

SEQ ID NO: 99 - protein, 10 aa, Dissostichus mawsoni
  MISC_FEATURE: Amino acid sequence of the Dissostichus mawsoni (Antarctic toothfish) NKBa peptide
EMNDIFVGLM

SEQ ID NO: 100 - protein, 13 aa, Sebastes rastrelliger
  MISC_FEATURE: Amino acid sequence of the Sebastes rastrelliger (grass rockfish) NKF peptide
YSDLDYDSFVGLM

SEQ ID NO: 101 - protein, 10 aa, Sebastes rastrelliger
  MISC_FEATURE: Amino acid sequence of the Sebastes rastrelliger (grass rockfish) NKBa peptide
EMHDIFVGLM

SEQ ID NO: 102 - protein, 13 aa, Gadus morhua
  MISC_FEATURE: Amino acid sequence of the Gadus morhua (Atlantic cod) NKF peptide
SSDLDYDSFVGLM

SEQ ID NO: 103 - protein, 13 aa, Boreogadus saida
  MISC_FEATURE: Amino acid sequence of the Arctic cod (Boreogadus saida) NKF peptide
FSDLDYDSFVGLM

SEQ ID NO: 104 - protein, 10 aa, Boreogadus saida
  MISC_FEATURE: Amino acid sequence of the Arctic cod (Boreogadus saida) NKBa peptide
EMHDIFVGLM

SEQ ID NO: 105 - protein, 13 aa, Osmerus mordax
  MISC_FEATURE: Amino acid sequence of the Osmerus mordax (rainbow smelt) NKF peptide
YSDVDYDSFVGLM

SEQ ID NO: 106 - protein, 10 aa, Osmerus mordax
  MISC_FEATURE: Amino acid sequence of the Osmerus mordax (rainbow smelt)
EMHDIFVGLM